(19) United States
(12) Patent Application Publication
(10) Pub. No.: US 2025/0230188 A1
(43) Pub. Date: Jul. 17, 2025

(54) METAGENOME-GUIDED BIOSYNTHESIS AND COMPOUNDS AND METHODS OF USE THEREOF

(71) Applicant: The Rockefeller University, New York, NY (US)

(72) Inventor: Sean Brady, New York, NY (US)

(21) Appl. No.: 18/853,774

(22) PCT Filed: Apr. 4, 2023
(86) PCT No.: PCT/US2023/065294

§ 371 Date: Oct. 3, 2024

Related U.S. Application Data
(60) Provisional application No. 63/327,060, filed on Apr. 4, 2022.

(51) Int. Cl. C07K 7/06; A61K 38/00; A61K 45/06; A61P 31/04; C12P 13/02; C12P 15/00

(52) U.S. Cl. CPC C07K 7/06; A61K 45/06; A61P 31/04; C12P 13/02; C12P 15/00; A61K 38/00

(57) ABSTRACT

The present invention provides methods, compositions, and articles of manufacture useful for the prophylactic and therapeutic amelioration and treatment of gram-positive bacteria, and related conditions. The present invention provides compositions and methods incorporating and utilizing antibiotics represented by Formulae (I)-(IX) or derivatives or variants thereof.

### CROSS-REFERENCE TO RELATED APPLICATIONS

This application claims priority to and the benefit of U.S. Provisional Application No. 63/327,060, filed Apr. 4, 2022, the disclosure of which is incorporated herein by reference in its entirety.

### STATEMENT OF GOVERNMENT FUNDING

This invention was made with government support under 1U19AI142731 and 5R35GM122559 awarded by the National Institutes of Health. The government has certain rights in the invention.

### BACKGROUND OF THE INVENTION

Natural products have been a rewarding source of therapeutically useful antibiotics (Baker D D et al., 2007, Nat Prod Rep, 24, 1225-1244; Butler M S et al., 2014, Nat Prod Rep, 31, 1612-1661; Newman D J et al., 2000, Nat Prod Rep, 17:215-234). The discovery of a new class of natural antibiotics is often followed by significant synthetic efforts to identify analogs with improved activity (Jones J A et al., 2016, Medchemcomm, 7:1694-1715; Smith P A et al. 2018, Nature, 561:189-194; Parkinson E I et al., 2015, Nat Commun, 6:6947). At least initially, these medicinal chemistry studies tend to be carried out in an ad hoc fashion with little direction as to where or how to modify the natural structure to yield improvements.

Thus, there is a need in the art for new compositions and methods for treating infections. The present invention satisfies the need in the art.

### SUMMARY OF THE INVENTION

The present invention provides, in part, a compound represented by Formula (I)

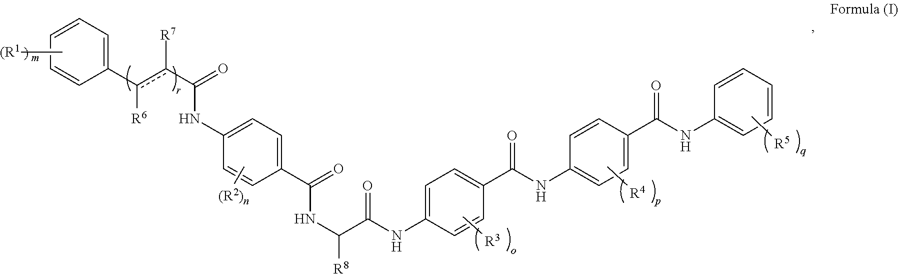

or a racemate, an enantiomer, a diastereomer, a pharmaceutically acceptable salt, or a derivative thereof.

In some embodiments, each R¹, R², R³, R⁴, R⁵, R⁶, R⁷, and R⁸ is independently selected from hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. In some embodiments, each R¹, R², R³, R⁴, R⁵, R⁶, R⁷, and R⁸ is independently selected from hydrogen, alkyl, amino, amido, hydroxyl, hydroxyalkyl, carboxyl, —CN, or any combination thereof. In some embodiments, each R¹, R², R³, R⁴, R⁵, R⁶, R⁷, and R⁸ is independently selected from hydrogen, methyl, amino, hydroxyl, carboxyl,

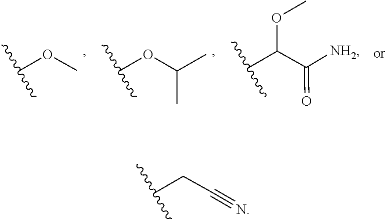

In some embodiments, each m and q independently represents an integer of 0 to 5.

In some embodiments, each n, o, and p independently represents an integer of 0 to 4.

In some embodiments, r represents an integer of 0 or 1.

In some embodiments, the compound represented by Formula (I) is a compound selected from:

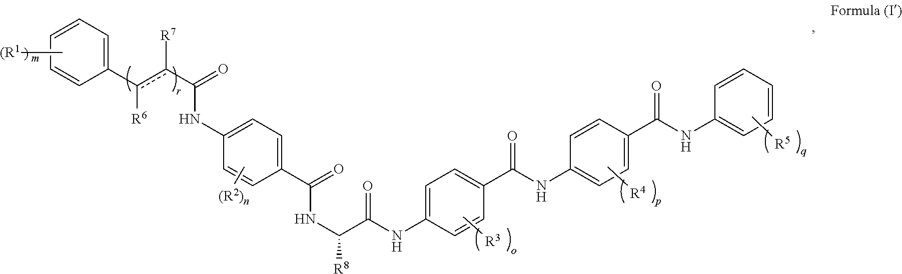

or a racemate, an enantiomer, a diastereomer, a pharmaceutically acceptable salt, or a derivative thereof, or

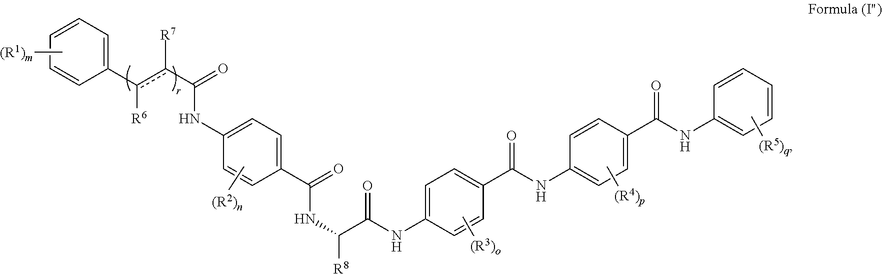

or a racemate, an enantiomer, a diastereomer, a pharmaceutically acceptable salt, or a derivative thereof.

In some embodiments, the compound represented by Formula (I) is a compound selected from:

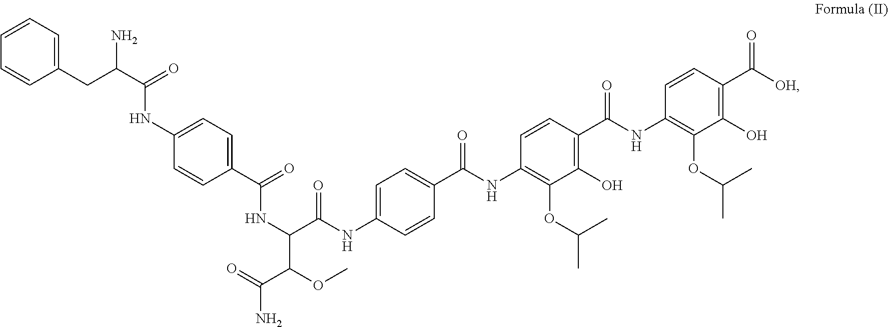 [SMILES: COC(C(N)=O)C(NC(=O)c1ccc(NC(=O)C(N)Cc2ccccc2)cc1)C(=O)Nc1ccc(C(=O)Nc2ccc(C(=O)Nc3ccc(C(=O)O)c(O)c3OC(C)C)c(O)c2OC(C)C)cc1]

or a racemate, an enantiomer, a diastereomer, a pharmaceutically acceptable salt, or a derivative thereof,

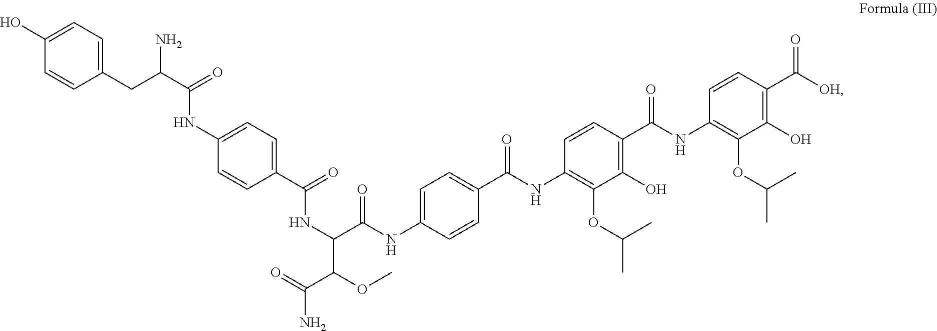 [SMILES: COC(C(N)=O)C(NC(=O)c1ccc(NC(=O)C(N)Cc2ccc(O)cc2)cc1)C(=O)Nc1ccc(C(=O)Nc2ccc(C(=O)Nc3ccc(C(=O)O)c(O)c3OC(C)C)c(O)c2OC(C)C)cc1]

or a racemate, an enantiomer, a diastereomer, a pharmaceutically acceptable salt, or a derivative thereof,

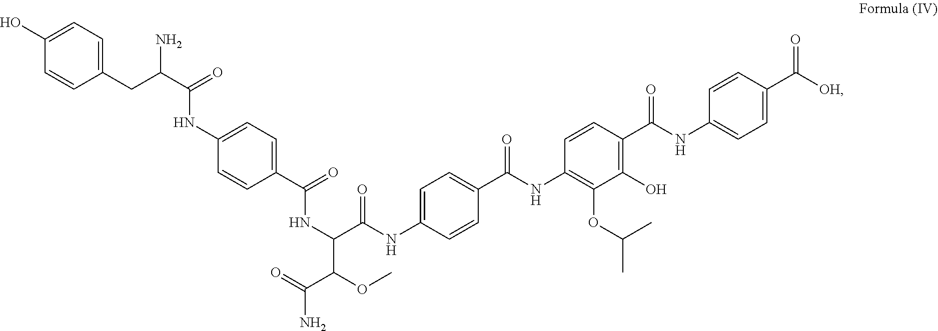 [SMILES: COC(C(N)=O)C(NC(=O)c1ccc(NC(=O)C(N)Cc2ccc(O)cc2)cc1)C(=O)Nc1ccc(C(=O)Nc2ccc(C(=O)Nc3ccc(C(=O)O)cc3)c(O)c2OC(C)C)cc1]

or a racemate, an enantiomer, a diastereomer, a pharmaceutically acceptable salt, or a derivative thereof,

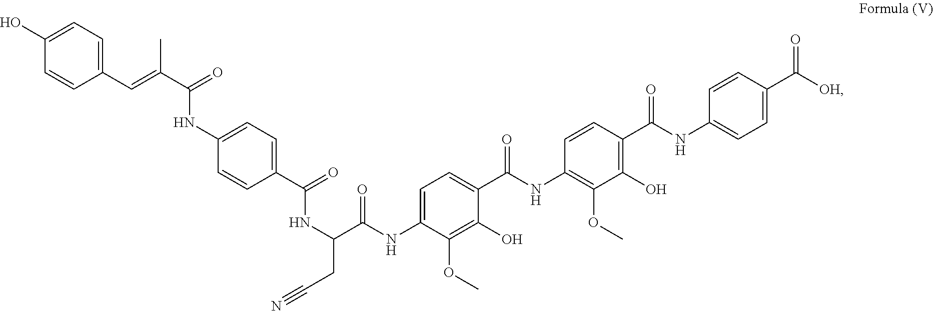 [SMILES: COc1c(NC(=O)c2ccc(NC(=O)C(CC#N)NC(=O)c3ccc(NC(=O)/C(C)=C/c4ccc(O)cc4)cc3)c(OC)c2O)ccc(C(=O)Nc2ccc(C(=O)O)cc2)c1O]

or a racemate, an enantiomer, a diastereomer, a pharmaceutically acceptable salt, or a derivative thereof,

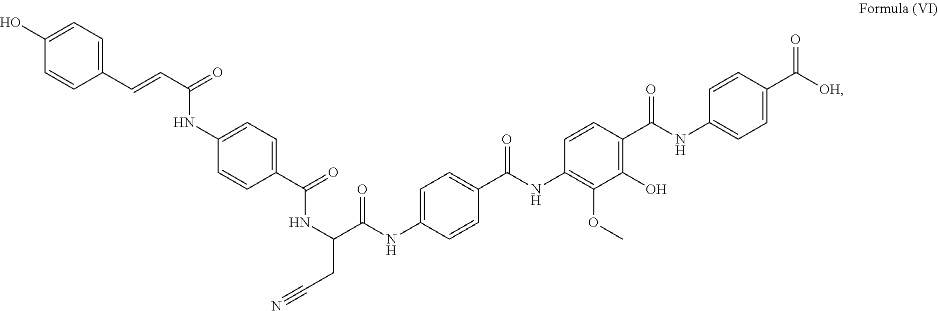 [SMILES: COc1c(NC(=O)c2ccc(NC(=O)C(CC#N)NC(=O)c3ccc(NC(=O)/C=C/c4ccc(O)cc4)cc3)cc2)ccc(C(=O)Nc2ccc(C(=O)O)cc2)c1O]

or a racemate, an enantiomer, a diastereomer, a pharmaceutically acceptable salt, or a derivative thereof,

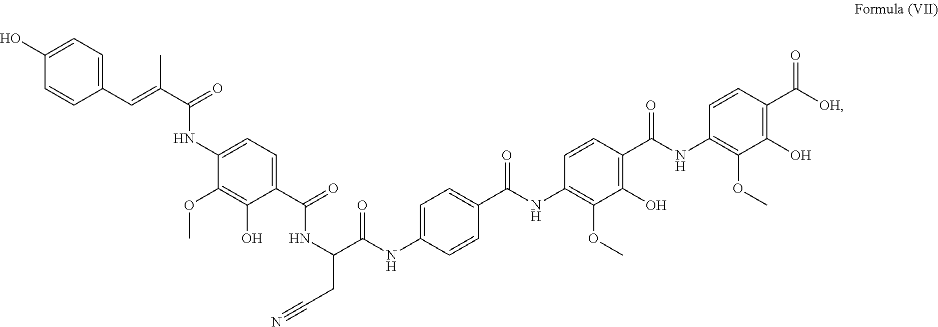 [SMILES: COc1c(NC(=O)c2ccc(NC(=O)c3ccc(NC(=O)C(CC#N)NC(=O)c4ccc(NC(=O)/C(C)=C/c5ccc(O)cc5)c(OC)c4O)cc3)c(OC)c2O)ccc(C(=O)O)c1O]

or a racemate, an enantiomer, a diastereomer, a pharmaceutically acceptable salt, or a derivative thereof,

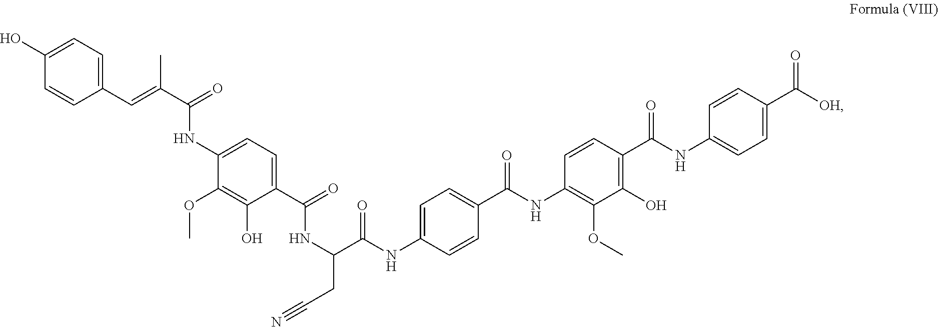 [SMILES: COc1c(NC(=O)/C(C)=C/c2ccc(O)cc2)ccc(C(=O)NC(CC#N)C(=O)Nc2ccc(C(=O)Nc3ccc(C(=O)Nc4ccc(C(=O)O)cc4)c(O)c3OC)cc2)c1O]

or a racemate, an enantiomer, a diastereomer, a pharmaceutically acceptable salt, or a derivative thereof, or

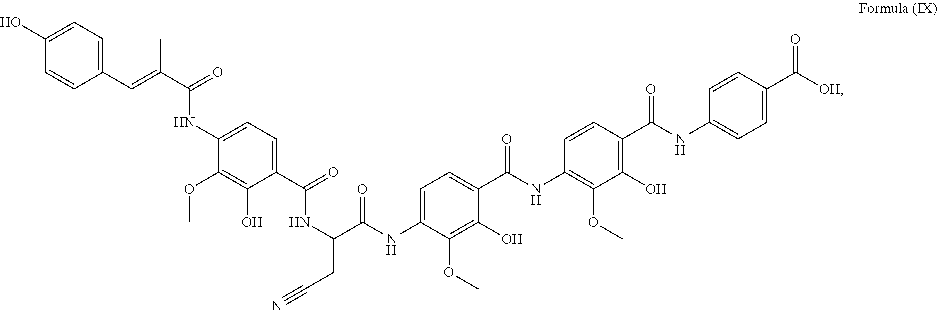 [SMILES: COc1c(NC(=O)/C(C)=C/c2ccc(O)cc2)ccc(C(=O)NC(CC#N)C(=O)Nc2ccc(C(=O)Nc3ccc(C(=O)Nc4ccc(C(=O)O)cc4)c(O)c3OC)c(O)c2OC)c1O]

or a racemate, an enantiomer, a diastereomer, a pharmaceutically acceptable salt, or a derivative thereof.

In some embodiments, the compound represented by Formula (I) is a compound selected from:

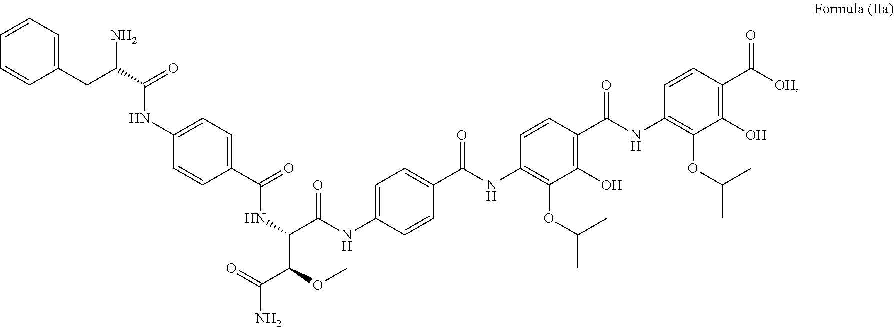

or a racemate, an enantiomer, a diastereomer, a pharmaceutically acceptable salt, or a derivative thereof,

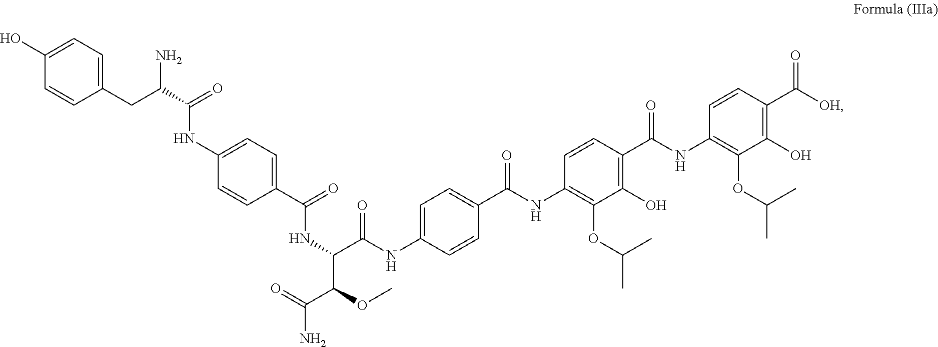

or a racemate, an enantiomer, a diastereomer, a pharmaceutically acceptable salt, or a derivative thereof,

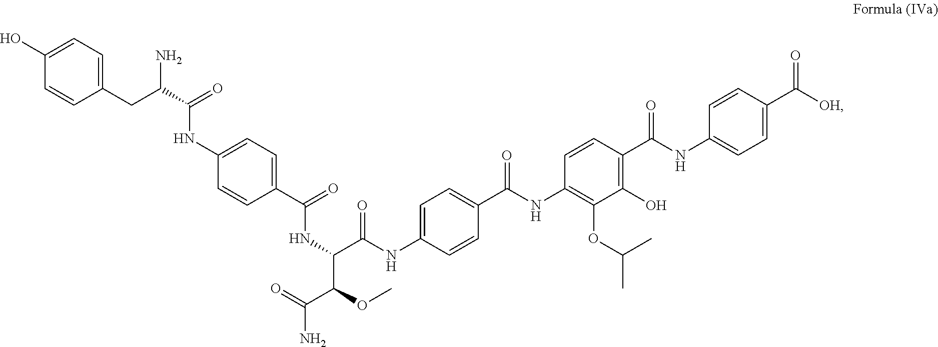

or a racemate, an enantiomer, a diastereomer, a pharmaceutically acceptable salt, or a derivative thereof,

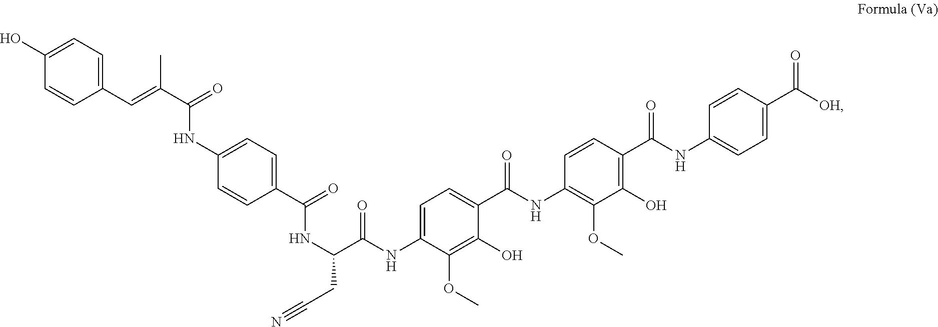 [SMILES: COc1c(NC(=O)c2ccc(NC(=O)[C@H](CC#N)NC(=O)c3ccc(NC(=O)/C(C)=C/c4ccc(O)cc4)cc3)c(OC)c2O)ccc(C(=O)Nc2ccc(C(=O)O)cc2)c1O]

or a racemate, an enantiomer, a diastereomer, a pharmaceutically acceptable salt, or a derivative thereof,

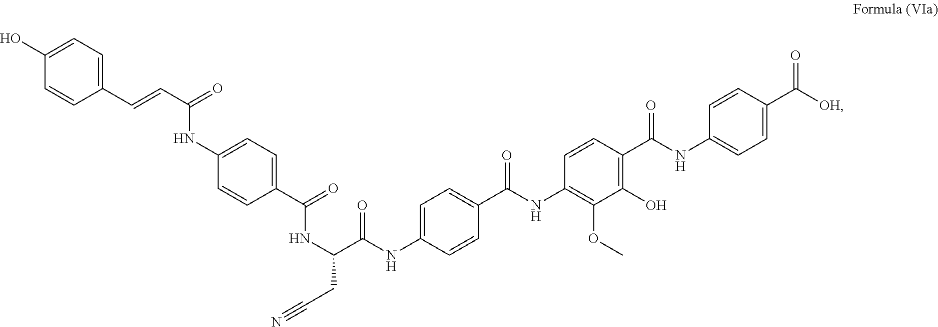 [SMILES: COc1c(NC(=O)c2ccc(NC(=O)[C@H](CC#N)NC(=O)c3ccc(NC(=O)/C=C/c4ccc(O)cc4)cc3)cc2)ccc(C(=O)Nc2ccc(C(=O)O)cc2)c1O]

or a racemate, an enantiomer, a diastereomer, a pharmaceutically acceptable salt, or a derivative thereof,

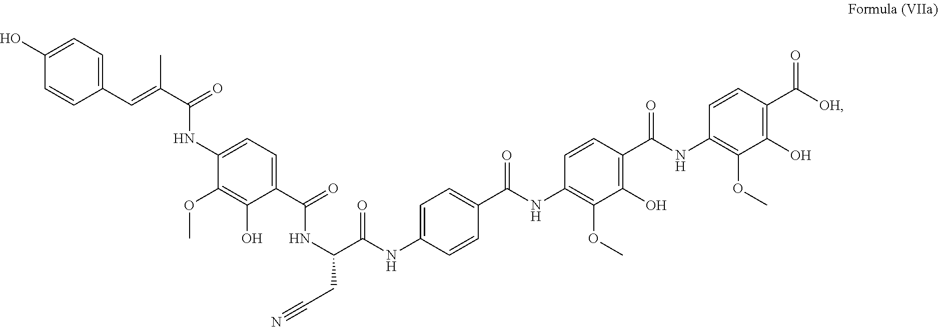 [SMILES: COc1c(NC(=O)c2ccc(NC(=O)c3ccc(NC(=O)[C@H](CC#N)NC(=O)c4ccc(NC(=O)/C(C)=C/c5ccc(O)cc5)c(OC)c4O)cc3)c(OC)c2O)ccc(C(=O)O)c1O]

or a racemate, an enantiomer, a diastereomer, a pharmaceutically acceptable salt, or a derivative thereof,

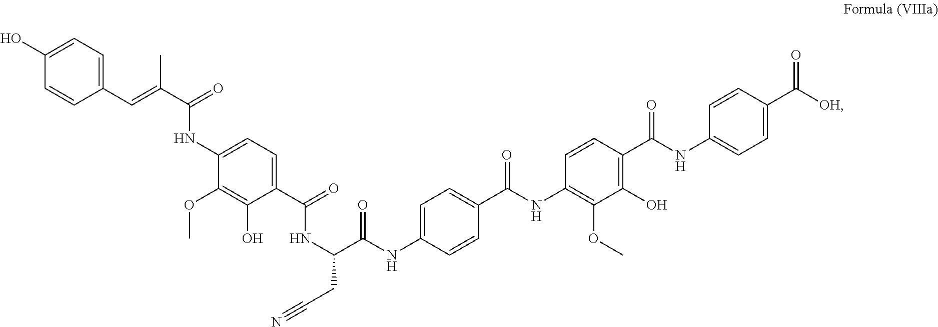 [SMILES: COc1c(NC(=O)/C(C)=C/c2ccc(O)cc2)ccc(C(=O)N[C@@H](CC#N)C(=O)Nc2ccc(C(=O)Nc3ccc(C(=O)Nc4ccc(C(=O)O)cc4)c(O)c3OC)cc2)c1O]

or a racemate, an enantiomer, a diastereomer, a pharmaceutically acceptable salt, or a derivative thereof, or

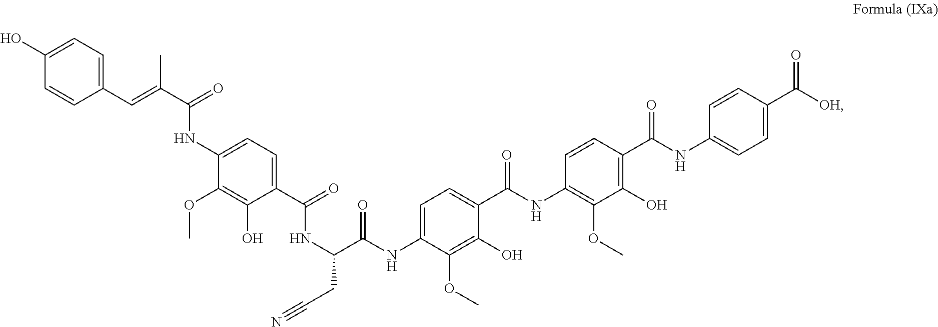 [SMILES: COc1c(NC(=O)/C(C)=C/c2ccc(O)cc2)ccc(C(=O)N[C@@H](CC#N)C(=O)Nc2ccc(C(=O)Nc3ccc(C(=O)Nc4ccc(C(=O)O)cc4)c(O)c3OC)c(O)c2OC)c1O]

or a racemate, an enantiomer, a diastereomer, a pharmaceutically acceptable salt, or a derivative thereof.

In one aspect, the present invention also provides a composition comprising one or more compounds of the present invention (i.e., compounds represented by Formulae (I)-(IX), including Formulae (I′), (II″), and (Ia)-(IXa), or a racemate, an enantiomer, a diastereomer, a pharmaceutically acceptable salt, or a derivative thereof).

In one embodiment, the composition is a pharmaceutical composition.

In another aspect, the present invention provides a nucleic acid encoding at least one compound of the present invention (i.e., compounds represented by Formulae (I)-(IX), including Formulae (I′), (II″), and (Ia)-(IXa), or a racemate, an enantiomer, a diastereomer, a pharmaceutically acceptable salt, or a derivative thereof). In one embodiment, the nucleic acid is an isolated nucleic acid.

In another aspect, the present invention provides a genetically engineered cell. In various embodiments, the cell expresses one or more compounds of the present invention (i.e., compounds represented by Formulae (I)-(IX), including Formulae (I′), (II″), and (Ia)-(IXa), or a racemate, an enantiomer, a diastereomer, a pharmaceutically acceptable salt, or a derivative thereof). In one embodiment, the cell is transformed with the nucleic acid of the present invention.

In another aspect, the present invention provides a method of treating or preventing a bacterial infection in a subject in need thereof. In some embodiments, the method comprises administering a composition comprising a compound of the present invention (i.e., compounds represented by Formulae (I)-(IX), including Formulae (I′), (II″), and (Ia)-(IXa), or a racemate, an enantiomer, a diastereomer, a pharmaceutically acceptable salt, or a derivative thereof) to the subject.

In some embodiments, the subject is exposed to or infected with a bacteria. In one embodiment, the bacteria is a gram positive bacteria. In one embodiment, the bacteria is a drug resistant bacteria.

In some embodiments, the method further comprises administering a second therapeutic. In one embodiment, the second therapeutic is an antibiotic.

In another aspect, the present invention provides a method of inhibiting the growth of or killing a bacterial cell. In some embodiments, the method comprises contacting the bacterial cell with a composition comprising a compound of the present invention (i.e., compounds represented by Formulae (I)-(IX), including Formulae (I′), (II″), and (Ia)-(IXa), or a racemate, an enantiomer, a diastereomer, a pharmaceutically acceptable salt, or a derivative thereof).

In another aspect, the present invention provides a method of biosynthesizing a gram-negative active compound. In some embodiments, the method comprises: a) generating a metagenome-derived congener biosynthetic gene cluster (BGC) comprising a nucleic acid molecule, wherein the nucleic acid molecule encodes the gram-negative active compound; b) providing the nucleic acid compound to a host; c) incubating the host in a growth medium; and d) isolating the gram-negative active compound from the host or the growth medium.

In some embodiments, the gram-negative active compound is a compound of the present invention (i.e., compounds represented by Formulae (I)-(IX), including Formulae (I′), (II″), and (Ia)-(IXa), or a racemate, an enantiomer, a diastereomer, a pharmaceutically acceptable salt, or a derivative thereof).

In another aspect, the present invention provides a method of generating a compound of the present invention. In some embodiments, the method comprises: a) reacting two or more compounds selected from:

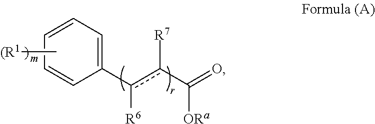

or a racemate, an enantiomer, a diastereomer, a pharmaceutically acceptable salt, or a derivative thereof,

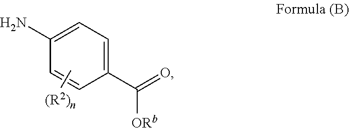

or a racemate, an enantiomer, a diastereomer, a pharmaceutically acceptable salt, or a derivative thereof,

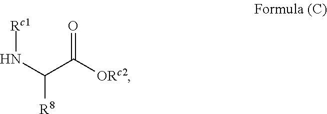

or a racemate, an enantiomer, a diastereomer, a pharmaceutically acceptable salt, or a derivative thereof,

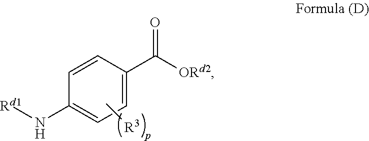

or a racemate, an enantiomer, a diastereomer, a pharmaceutically acceptable salt, or a derivative thereof,

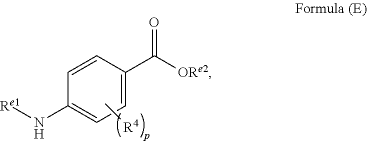

or a racemate, an enantiomer, a diastereomer, a pharmaceutically acceptable salt, or a derivative thereof, or

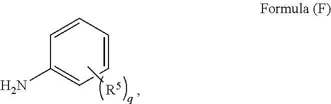

or a racemate, an enantiomer, a diastereomer, a pharmaceutically acceptable salt, or a derivative thereof.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A and FIG. 1B depicts a schematic representation of metagenome-guided medicinal chemistry overview. FIG. 1A depicts a schematic representation demonstrating that natural selection is indicated to result in congeners with varied potency, spectrum of activity, and response to resistance. Bioinformatic analysis of biosynthetic gene clusters (BGCs) that encode these congeners can guide the synthesis of improved antibiotics. FIG. 1B depicts a schematic representation demonstrating that to identify albicidin and cystobactamid congener BGCs, a domain sequences amplified from soil metagenomic libraries were searched for sequences that grouped with domains known to incorporate PABA. These sequences were used to guide the recovery of clones containing albicidin and cystobactamid congener BGCs.

FIG. 2, comprising FIG. 2A and FIG. 2B, depicts representative results demonstrating albicidin and cystobactamid congener BGCs isolated from soil metagenomic libraries and representative analysis used to predict synthetic bioinformatic natural product (syn-B3NP) targets. FIG. 2A depicts representative results demonstrating albicidin and cystobactamid congener BGCs isolated from soil metagenomic libraries. FIG. 2B depicts representative biosynthetic pathway for PABA34. Representative analysis used to predict syn-BNP targets.

FIG. 3, comprising FIG. 3A and FIG. 3B, depicts representative results demonstrating bioinformatically-derived structures and representative retrosynthetic analyses.

FIG. 3A depicts representative results demonstrating bioinformatically-derived congener structures. FIG. 3B depicts representative retrosynthetic analysis and synthetic monomers used in forward syntheses.

FIG. 4 depicts representative biosynthetic pathway for PABA34.

FIG. 5 depicts representative biosynthetic pathway for PABA95.

FIG. 6 depicts representative biosynthetic pathway for PABA157.

FIG. 7 depicts representative biosynthetic pathway for PABA48.

FIG. 8, comprising FIG. 8A through FIG. 8E, depicts representative results demonstrating that syn-BNPs have different resistance profiles. FIG. 8A depicts representative results demonstrating that activity of syn-BNPs against ciprofloxacin-resistant E. coli. FIG. 8B depicts representative results demonstrating representative MIC fold difference between E. coli that either expresses or does not express AlbD. FIG. 8C depicts representative results of HPLC analysis of PABA34 and albicidin digested by AlbD. FIG. 8D depicts a schematic representation of AHMBA protection from AlbD cleavage. FIG. 8E depicts a schematic representation of PABA95 analog containing AHMBA that is protected from AlbD cleavage.

FIG. 9 depicts representative results of DNA gyrase supercoiling assay with PABA syn-BNPs or ciprofloxacin (CIP) performed at the indicated concentrations.

### DETAILED DESCRIPTION

The present invention is based, in part, on the unexpected discovery that metagenome-guided medicinal chemistry yielded novel and improved gram-negative active albicidin- and cystobactamid-type compounds as antibiotics which have activity against multidrug resistant pathogens. In one embodiment, the present invention provides compounds (e.g., compounds represented by Formulae (I)-(IX)) or a therapeutic compound comprising a desired activity. In one embodiment, the compound is an antibiotic. In one embodiment, the antibiotic compound of the invention can be used in the treatment of bacterial infections. In one embodiment, the antibiotic compound of the invention can be used in the treatment of gram positive bacterial infections. In certain embodiments, the use of the antibiotic compound of the invention in the treatment of bacterial infections optionally includes a pharmaceutically acceptable carrier, excipient, or adjuvant.

In one embodiment, the compound of the present invention can be biosynthesized via heterologous expression of a biosynthetic gene. Thus, in one aspect, the invention provides methods for synthesizing one or more compounds of the present invention. In one embodiment, the invention provides a nucleic acid encoding one or more compounds of the present invention. In one embodiment, the nucleic acid is an isolated nucleic acid. In one embodiment, the nucleic acid is transformed into a cell.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

An “amino terminus modification group” refers to any molecule that can be attached to the amino terminus of a polypeptide. Similarly, a “carboxy terminus modification group” refers to any molecule that can be attached to the carboxy terminus of a polypeptide. Terminus modification groups include but are not limited to various water soluble polymers, peptides or proteins such as serum albumin, or other moieties that increase serum half-life of peptides.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The term, “biologically active” or “bioactive” can mean, but is in no way limited to, the ability of an agent or compound to effectuate a physiological change or response. The response may be detected, for example, at the cellular level, for example, as a change in growth and/or viability, gene expression, protein quantity, protein modification, protein activity, or combination thereof; at the tissue level; at the systemic level; or at the organism level. For example, as used herein, biologically active molecules include but are not limited to any substance intended for diagnosis, cure, mitigation, treatment, or prevention of disease in humans or other animals, or to otherwise enhance physical or mental well-being of humans or animals. Examples of biologically active molecules include, but are not limited to, peptides, proteins, enzymes, small molecule drugs, dyes, lipids, nucleosides, oligonucleotides, cells, viruses, liposomes, microparticles and micelles. Classes of biologically active agents that are suitable for use with the invention include, but are not limited to, antibiotics, fungicides, anti-viral agents, anti-inflammatory agents, anti-tumor agents, cardiovascular agents, anti-anxiety agents, hormones, growth factors, steroidal agents, and the like.

The term “conservative mutations” refers to the substitution, deletion or addition of nucleic acids that alter, add or delete a single amino acid or a small number of amino acids in a coding sequence where the nucleic acid alterations result in the substitution of a chemically similar amino acid. Amino acids that may serve as conservative substitutions for each other include the following:
Basic: Arginine (R), Lysine (K), Histidine (H);
Acidic: Aspartic acid (D), Glutamic acid (E);
Neutral: Asparagine (N), Cysteine (C), Glutamine (Q), Methionine (M), Serine (S), Threonine (T);
Aliphatic: Alanine (A), Valine (V), Leucine (L), Isoleucine (I), Glycine (G);
Hydrophobic: Aromatic: Phenylalanine (F), Tyrosine (Y), Tryptophan (W);
Sulfur-containing: Methionine (M), Cysteine (C)
Hydroxyl: Serine (S), Threonine (T);
Aminde: Asparagine (N), Glutamine (Q).

In addition, sequences that differ by conservative variations are generally homologous. In some instances, the following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

As used herein, “derivatives” are compositions formed from the native compounds either directly, by modification, or by partial substitution. As used herein, “analogs” are compositions that have a structure similar to, but not identical to, the native compound.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “amino acid”, “amino acidic monomer”, or “amino acid residue” refer to any of the twenty naturally occurring amino acids including synthetic amino acids with unnatural side chains and including both D and L optical isomers.

In the context of the invention, term “natural amino acid” means any amino acid which is found naturally in vivo in a living being. Natural amino acids therefore include amino acids coded by mRNA incorporated into proteins during translation but also other amino acids found naturally in vivo which are a product or by-product of a metabolic process, such as for example ornithine which is generated by the urea production process by arginase from L-arginine. In the invention, the amino acids used can therefore be natural or not. Namely, natural amino acids generally have the L configuration but also, according to the invention, an amino acid can have the L or D configuration.

A “non-naturally encoded amino acid” refers to an amino acid that is not one of the 20 common amino acids or pyrolysine or selenocysteine. The term “non-naturally encoded amino acid” includes, but is not limited to, amino acids that occur naturally by modification of a naturally encoded amino acid (including but not limited to, the 20 common amino acids or pyrolysine and selenocysteine) but are not themselves incorporated into a growing polypeptide chain by the translation complex. Examples of naturally-occurring amino acids that are not naturally-encoded include, but are not limited to, N-acetylglucosaminyl-L-serine, N-acetylglucosaminyl-L-threonine, and O-phosphotyrosine.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Parenteral” administration of a composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. Furthermore, peptides of the invention may include amino acid mimentics, and analogs. Recombinant forms of the peptides can be produced according to standard methods and protocols which are well known to those of skill in the art, including for example, expression of recombinant proteins in prokaryotic and/or eukaryotic cells followed by one or more isolation and purification steps, and/or chemically synthesizing peptides or portions thereof using a peptide sythesizer.

Conventional notation is used herein to portray polypeptide sequences: the left-hand end of a polypeptide sequence is the amino-terminus; the right-hand end of a polypeptide sequence is the carboxyl-terminus.

As used herein, a “peptidomimetic” is a compound containing non-peptidic structural elements that is capable of mimicking the biological action of a parent peptide. A peptidomimetic may or may not comprise peptide bonds.

The term “recombinant polypeptide” as used herein is defined as a polypeptide produced by using recombinant DNA methods. A host cell that comprises a recombinant polynucleotide is referred to as a “recombinant host cell.” A gene which is expressed in a recombinant host cell wherein the gene comprises a recombinant polynucleotide, produces a “recombinant polypeptide.”

The term “pharmacological composition,” “therapeutic composition,” “therapeutic formulation” or “pharmaceutically acceptable formulation” can mean, but is in no way limited to, a composition or formulation that allows for the effective distribution of an agent provided by the invention, which is in a form suitable for administration to the physical location most suitable for their desired activity, e.g., systemic administration.

Non-limiting examples of agents suitable for formulation with the, e.g., compounds provided by the instant invention include: cinnamoyl, PEG, phospholipids or lipophilic moieties, phosphorothioates, β-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

The term “pharmaceutically acceptable” or “pharmacologically acceptable” can mean, but is in no way limited to, entities and compositions that do not produce an adverse, allergic or other untoward reaction when administered to an animal, or a human, as appropriate.

The term “pharmaceutically acceptable carrier” or “pharmacologically acceptable carrier” can mean, but is in no way limited to, any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “compound,” as used herein, unless otherwise indicated, refers to any specific chemical compound disclosed herein. In one embodiment, the term also refers to stereoisomers and/or optical isomers (including racemic mixtures) or enantiomerically enriched mixtures of disclosed compounds.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, or other variant of the reference molecule.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and includes straight, branched chain, or cyclic substituent groups. Examples include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. The term “alkyl,” unless otherwise noted, is also meant to include those derivatives of alkyl defined in more detail below, such as “heteroalkyl”, “haloalkyl” and “homoalkyl”.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C—N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkylene” by itself or as part of another molecule means a divalent radical derived from an alkane, as exemplified by (—CH₂—)ₙ. By way of example only, such groups include, but are not limited to, groups having 24 or fewer carbon atoms such as the structures —CH₂CH₂— and —CH₂CH₂CH₂CH₂—. The term “alkylene,” unless otherwise noted, is also meant to include those groups described below as “heteroalkylene.”

As used herein, the terms “alkoxy,” “alkylamino” and “alkylthio” are used in their conventional sense, and refer to alkyl groups linked to molecules via an oxygen atom, an amino group, a sulfur atom, respectively.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

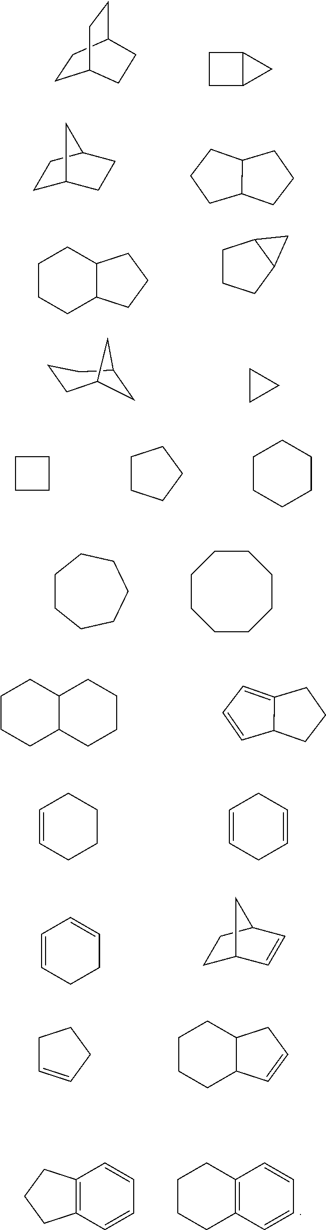

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, Si, P, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

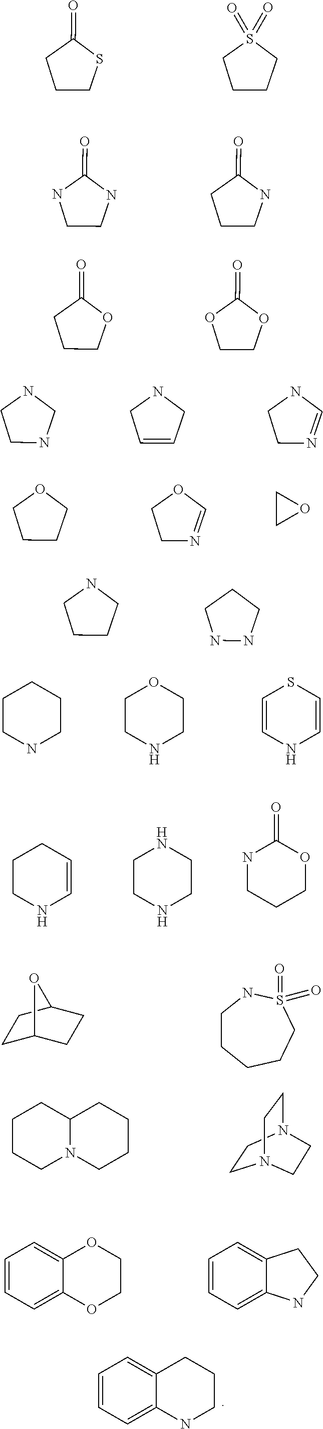

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

As used herein, the term “aromatic” refers to a carbocycle or heterocycle with one or more polyunsaturated rings and having aromatic character, i.e. having (4n+2) delocalized Tc (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₄)alkyl” means a functional group wherein a one to three carbon alkylene chain is attached to an aryl group, e.g., —CH₂CH₂-phenyl. Preferred is aryl-CH₂— and aryl-CH(CH₃)—. The term “substituted aryl-(C₁-C₄)alkyl” means an aryl-(C₁-C₄)alkyl functional group in which the aryl group is substituted. Preferred is substituted aryl(CH₂)—. Similarly, the term “heteroaryl-(C₁-C₄)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₄)alkyl” means a heteroaryl-(C₁-C₄)alkyl functional group in which the heteroaryl group is substituted. Preferred is substituted heteroaryl-(CH₂)—.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “amino aryl” refers to an aryl moiety which contains an amino moiety. Such amino moieties may include, but are not limited to primary amines, secondary amines, tertiary amines, masked amines, or protected amines. Such tertiary amines, masked amines, or protected amines may be converted to primary amine or secondary amine moieties. Additionally, the amine moiety may include an amine-like moiety which has similar chemical characteristics as amine moieties, including but not limited to chemical reactivity.

As used herein, the term “substituted” means that an atom or group of atoms has replaced hydrogen as the substituent attached to another group. For aryl, aryl-(C₁-C₄)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁-6 alkyl, —OH, C₁₋₆ alkoxy, halo, amino, acetamido and nitro. In yet another embodiment, the substituents are independently selected from the group consisting of C₁-6 alkyl, C₁-6 alkoxy, halo, acetamido, and nitro. As used herein, where a substituent is an alkyl or alkoxy group, the carbon chain may be branched, straight or cyclic, with straight being preferred.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]2, —OC(═O)N[substituted or unsubstituted alkyl]2, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]2, and —C(NH₂)[substituted or unsubstituted alkyl]2. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁-6 alkoxy, halo, amino, acetamido, oxo and nitro. In yet another embodiment, the substituents are independently selected from the group consisting of C₁-6 alkyl, C₁-6 alkoxy, halo, acetamido, and nitro. As used herein, where a substituent is an alkyl or alkoxy group, the carbon chain may be branched, straight or cyclic.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the unexpected discovery that metagenome-guided medicinal chemistry yielded novel and improved gram-negative active albicidin- and cystobactamid-type compounds as antibiotics, which had activity against multidrug resistant pathogens. In one embodiment, the present invention provides compounds (e.g., compounds represented by Formulae (I)-(IX)) or a therapeutic compound comprising a desired activity. In one embodiment, the compound is an antibiotic. In one embodiment, the antibiotic compound of the invention can be used in the treatment of bacterial infections. In one embodiment, the antibiotic compound of the invention can be used in the treatment of gram positive bacterial infections. In certain embodiments, the use of the antibiotic compound of the invention in the treatment of bacterial infections optionally includes a pharmaceutically acceptable carrier, excipient, or adjuvant.

In one embodiment, the compound of the present invention can be biosynthesized via heterologous expression of a biosynthetic gene. Thus, in one aspect, the invention provides methods for synthesizing one or more compounds of the present invention. In one embodiment, the invention provides a nucleic acid encoding one or more compounds of the present invention. In one embodiment, the nucleic acid is an isolated nucleic acid. In one embodiment, the nucleic acid is transformed into a cell.

### Compounds of the Invention

In one aspect, the present invention provides novel metagenome-derived compounds. In some embodiments, the metagenome-derived compounds are albicidin- and cystobactamid-type compounds, or racemate, an enantiomer, a diastereomer thereof, a pharmaceutically acceptable salt, or a derivative thereof.

In one embodiment, the albicidin- and cystobactamid-type compounds are compounds having the structure of Formula (I)

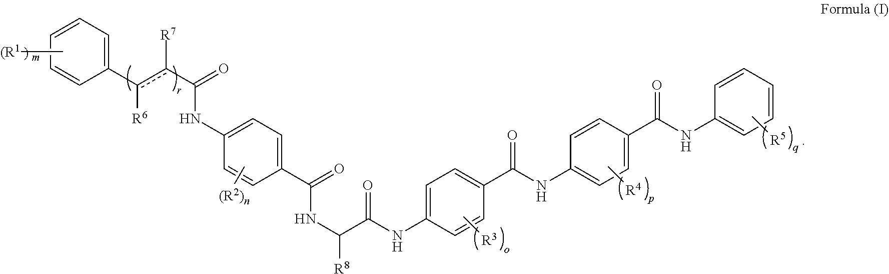

Thus, in one aspect, the present invention provides a compound having the structure of Formula (I), or racemate, an enantiomer, a diastereomer thereof, a pharmaceutically acceptable salt, or a derivative thereof.

In various embodiments, R¹ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof.

In various embodiments, R² is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof.

In various embodiments, R³ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof.

In various embodiments, R⁴ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof.

In various embodiments, R⁵ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof.

In various embodiments, R⁶ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof.

In various embodiments, R⁷ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof.

In various embodiments, R⁸ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof.

For example, in some embodiments, each R¹, R², R³, R⁴, R⁵, R⁶, R⁷, and R⁸ is independently hydrogen, alkyl, amino, amido, hydroxyl, hydroxyalkyl, carboxyl, —CN, or any combination thereof. In other embodiments, each R¹, R², R³, R⁴, R⁵, R⁶, R⁷, and R⁸ is independently hydrogen, methyl, amino, hydroxyl carboxyl,

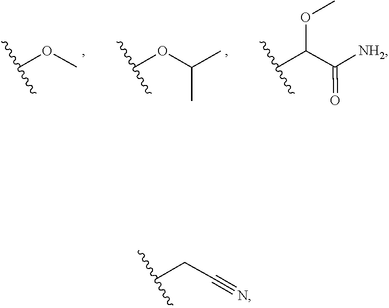

N or any combination thereof.

In various embodiments, m represents an integer of 0 to 5. For example, in one embodiment, m represents an integer 1. In one embodiment, m represents an integer 0.

In various embodiments, n represents an integer of 0 to 4. For example, in one embodiment, n represents an integer 2. In one embodiment, n represents an integer 0.

In various embodiments, o represents an integer of 0 to 4. For example, in one embodiment, o represents an integer 2. In one embodiment, o represents an integer 0.

In various embodiments, p represents an integer of 0 to 4. For example, in one embodiment, p represents an integer 2. In one embodiment, p represents an integer 0.

In various embodiments, q represents an integer of 0 to 5. For example, in one embodiment, q represents an integer 1. In one embodiment, q represents an integer 0.

In various embodiments, r represents an integer of 0 or 1. For example, in one embodiment, r represents an integer 1. In one embodiment, r represents an integer 0.

For example, in one embodiment, the compound represented by Formula (I) is a compound represented by Formula (II)

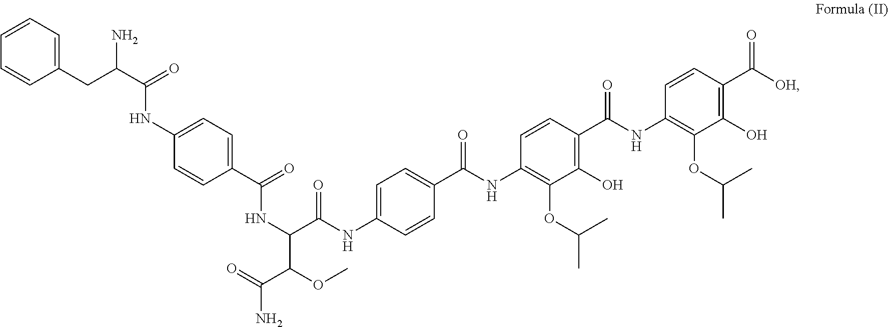 [SMILES: COC(C(N)=O)C(NC(=O)c1ccc(NC(=O)C(N)Cc2ccccc2)cc1)C(=O)Nc1ccc(C(=O)Nc2ccc(C(=O)Nc3ccc(C(=O)O)c(O)c3OC(C)C)c(O)c2OC(C)C)cc1]

or a racemate, an enantiomer, a diastereomer, a pharmaceutically acceptable salt, or a derivative thereof.

In one embodiment, the compound represented by Formula (I) is a compound represented by Formula (III)

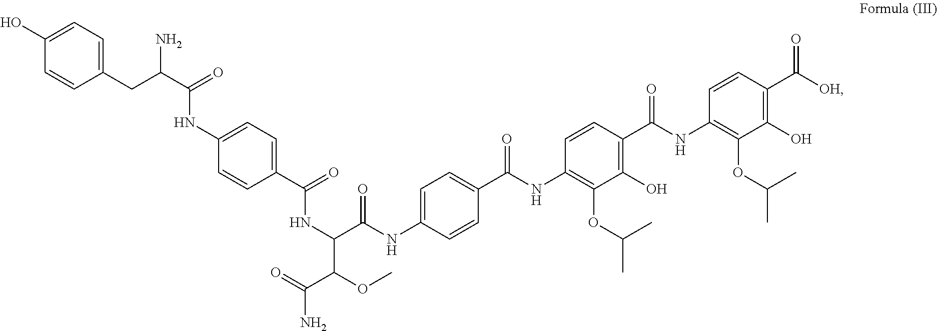 [SMILES: COC(C(N)=O)C(NC(=O)c1ccc(NC(=O)C(N)Cc2ccc(O)cc2)cc1)C(=O)Nc1ccc(C(=O)Nc2ccc(C(=O)Nc3ccc(C(=O)O)c(O)c3OC(C)C)c(O)c2OC(C)C)cc1]

or a racemate, an enantiomer, a diastereomer, a pharmaceutically acceptable salt, or a derivative thereof.

In one embodiment, the compound represented by Formula (I) is a compound represented by Formula (IV)

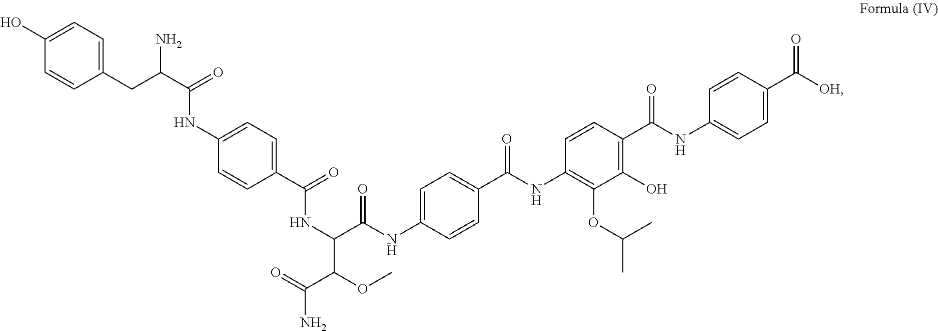 [SMILES: COC(C(N)=O)C(NC(=O)c1ccc(NC(=O)C(N)Cc2ccc(O)cc2)cc1)C(=O)Nc1ccc(C(=O)Nc2ccc(C(=O)Nc3ccc(C(=O)O)cc3)c(O)c2OC(C)C)cc1]

or a racemate, an enantiomer, a diastereomer, a pharmaceutically acceptable salt, or a derivative thereof.

In one embodiment, the compound represented by Formula (I) is a compound represented by Formula (V)

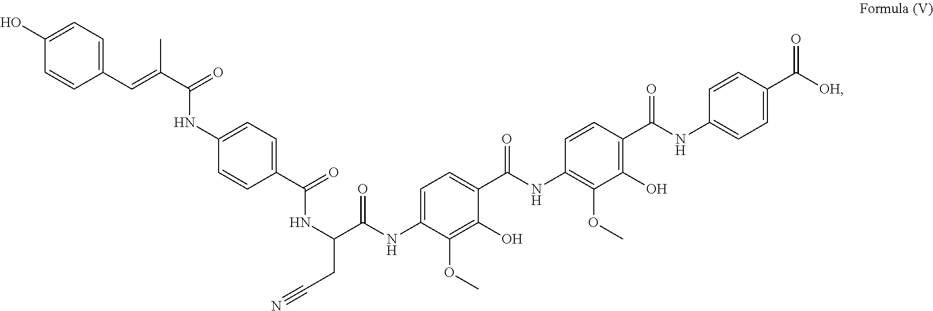 [SMILES: COc1c(NC(=O)c2ccc(NC(=O)C(CC#N)NC(=O)c3ccc(NC(=O)/C(C)=C/c4ccc(O)cc4)cc3)c(OC)c2O)ccc(C(=O)Nc2ccc(C(=O)O)cc2)c1O]

or a racemate, an enantiomer, a diastereomer, a pharmaceutically acceptable salt, or a derivative thereof.

In one embodiment, the compound represented by Formula (I) is a compound represented by Formula (VI)

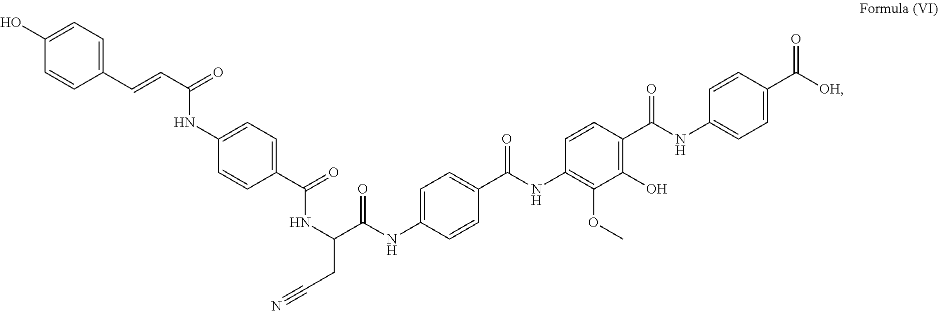 [SMILES: COc1c(NC(=O)c2ccc(NC(=O)C(CC#N)NC(=O)c3ccc(NC(=O)/C=C/c4ccc(O)cc4)cc3)cc2)ccc(C(=O)Nc2ccc(C(=O)O)cc2)c1O]

or a racemate, an enantiomer, a diastereomer, a pharmaceutically acceptable salt, or a derivative thereof.

In one embodiment, the compound represented by Formula (I) is a compound represented by Formula (VII)

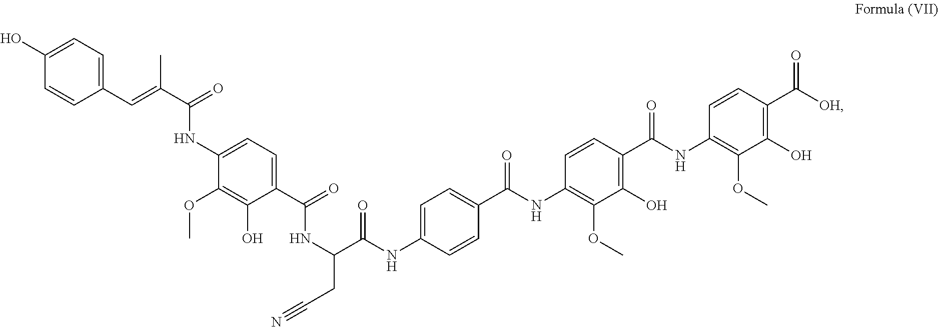 [SMILES: COc1c(NC(=O)c2ccc(NC(=O)c3ccc(NC(=O)C(CC#N)NC(=O)c4ccc(NC(=O)/C(C)=C/c5ccc(O)cc5)c(OC)c4O)cc3)c(OC)c2O)ccc(C(=O)O)c1O]

or a racemate, an enantiomer, a diastereomer, a pharmaceutically acceptable salt, or a derivative thereof.

In one embodiment, the compound represented by Formula (I) is a compound represented by Formula (VIII)

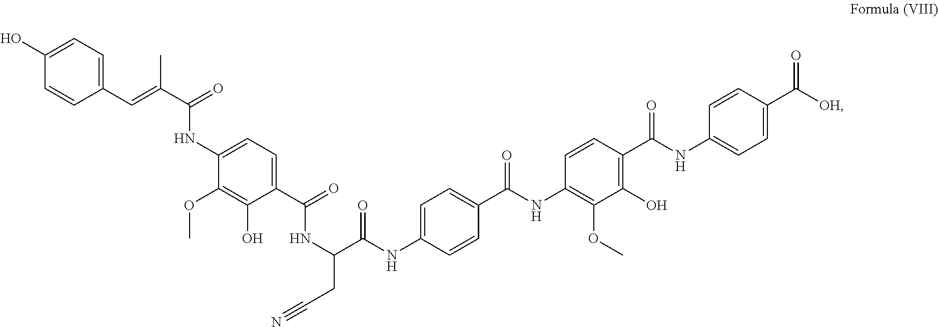 [SMILES: COc1c(NC(=O)/C(C)=C/c2ccc(O)cc2)ccc(C(=O)NC(CC#N)C(=O)Nc2ccc(C(=O)Nc3ccc(C(=O)Nc4ccc(C(=O)O)cc4)c(O)c3OC)cc2)c1O]

or a racemate, an enantiomer, a diastereomer, a pharmaceutically acceptable salt, or a derivative thereof.

In one embodiment, the compound represented by Formula (I) is a compound represented by Formula (IX)

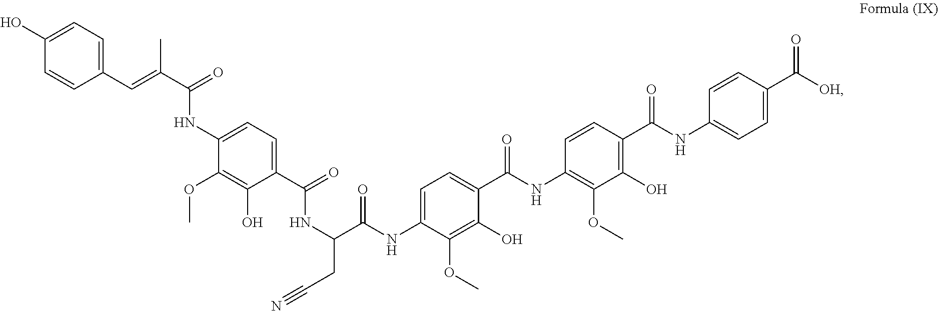 [SMILES: COc1c(NC(=O)/C(C)=C/c2ccc(O)cc2)ccc(C(=O)NC(CC#N)C(=O)Nc2ccc(C(=O)Nc3ccc(C(=O)Nc4ccc(C(=O)O)cc4)c(O)c3OC)c(O)c2OC)c1O]

or a racemate, an enantiomer, a diastereomer, a pharmaceutically acceptable salt, or a derivative thereof.

In some embodiments, the compound represented by Formula (I) is a compound represented by Formula (I′)

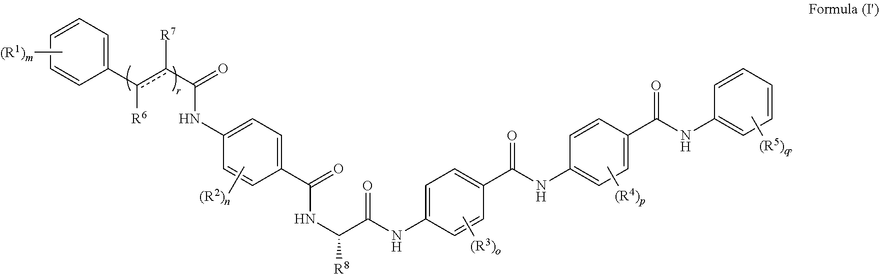

or a racemate, an enantiomer, a diastereomer, a pharmaceutically acceptable salt, or a derivative thereof.

In some embodiments, the compound represented by Formula (I) is a compound represented by Formula (I″)

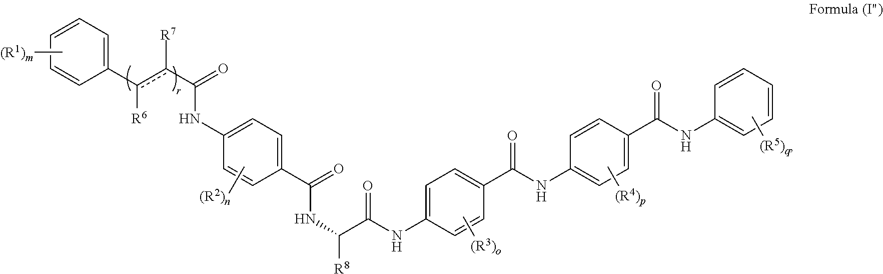

or a racemate, an enantiomer, a diastereomer, a pharmaceutically acceptable salt, or a derivative thereof.

For example, in one embodiment, the compound represented by Formula (I) is a compound represented by Formula (IIa)

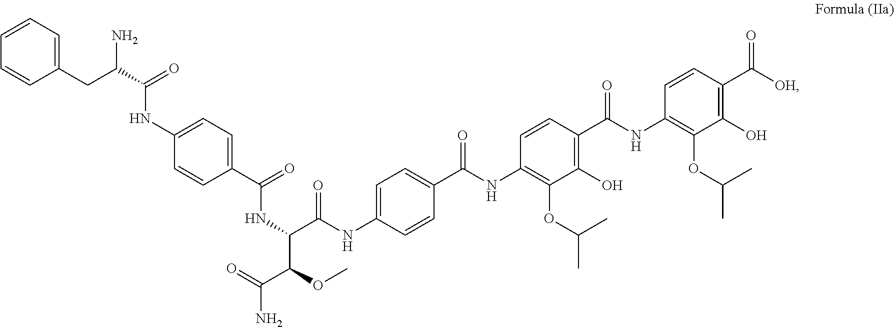

or a racemate, an enantiomer, a diastereomer, a pharmaceutically acceptable salt, or a derivative thereof.

In one embodiment, the compound represented by Formula (I) is a compound represented by Formula (IIIa)

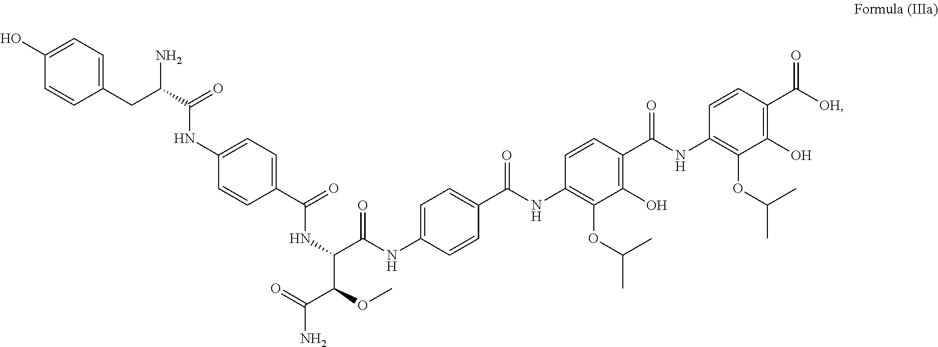 [SMILES: CO[C@@H](C(N)=O)[C@H](NC(=O)c1ccc(NC(=O)[C@@H](N)Cc2ccc(O)cc2)cc1)C(=O)Nc1ccc(C(=O)Nc2ccc(C(=O)Nc3ccc(C(=O)O)c(O)c3OC(C)C)c(O)c2OC(C)C)cc1]

or a racemate, an enantiomer, a diastereomer, a pharmaceutically acceptable salt, or a derivative thereof.

In one embodiment, the compound represented by Formula (I) is a compound represented by Formula (IVa)

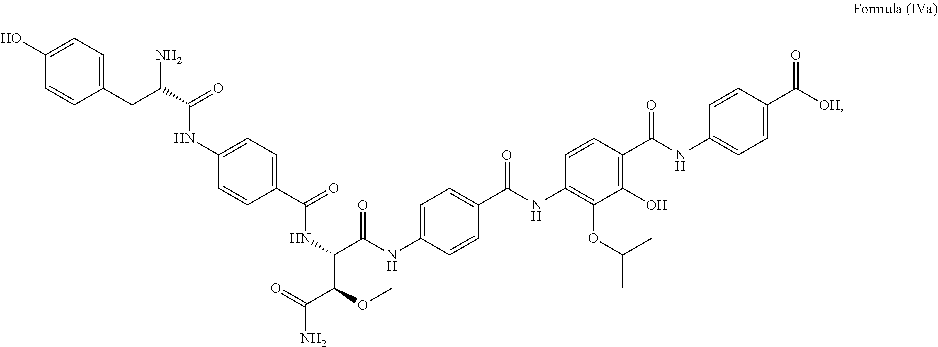

or a racemate, an enantiomer, a diastereomer, a pharmaceutically acceptable salt, or a derivative PG-3C thereof.

In one embodiment, the compound represented by Formula (I) is a compound represented by Formula (Va)

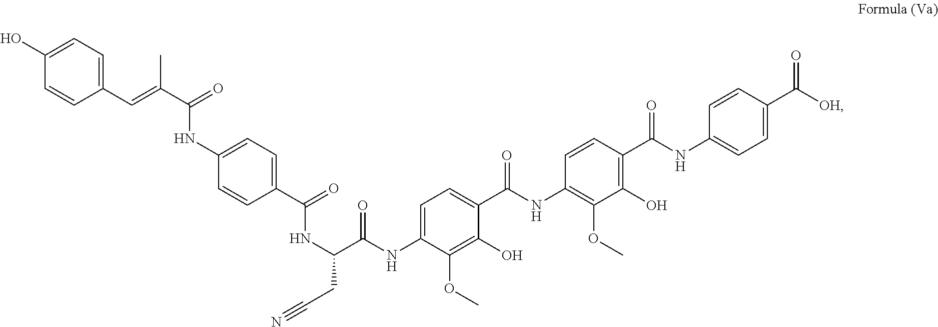 [SMILES: COc1c(NC(=O)c2ccc(NC(=O)[C@H](CC#N)NC(=O)c3ccc(NC(=O)/C(C)=C/c4ccc(O)cc4)cc3)c(OC)c2O)ccc(C(=O)Nc2ccc(C(=O)O)cc2)c1O]

or a racemate, an enantiomer, a diastereomer, a pharmaceutically acceptable salt, or a derivative thereof.

In one embodiment, the compound represented by Formula (I) is a compound represented by Formula (VIa)

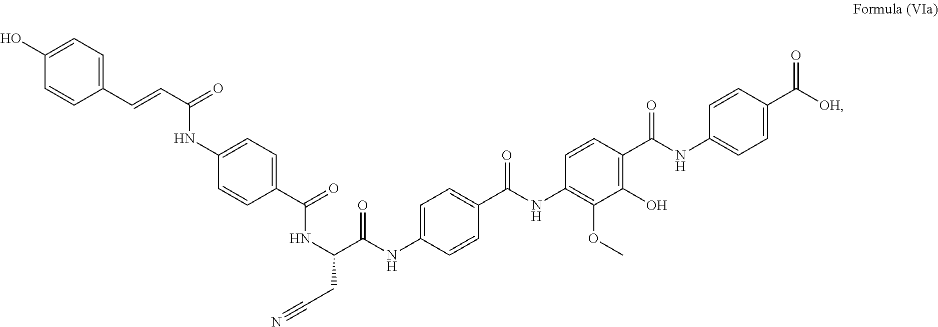 [SMILES: COc1c(NC(=O)c2ccc(NC(=O)[C@H](CC#N)NC(=O)c3ccc(NC(=O)/C=C/c4ccc(O)cc4)cc3)cc2)ccc(C(=O)Nc2ccc(C(=O)O)cc2)c1O]

or a racemate, an enantiomer, a diastereomer, a pharmaceutically acceptable salt, or a derivative thereof.

In one embodiment, the compound represented by Formula (I) is a compound represented by Formula (VIIa)

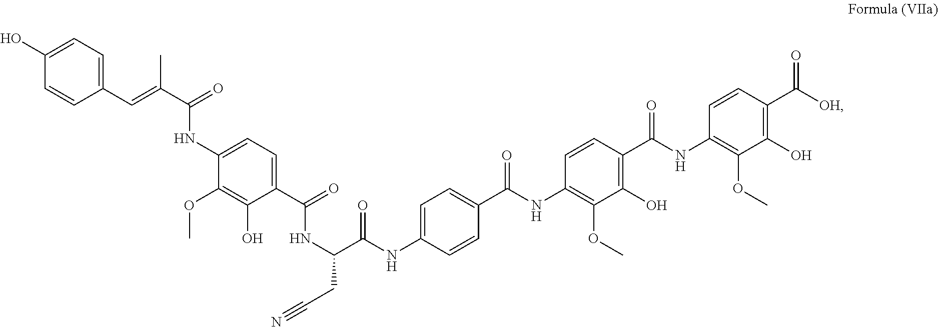 [SMILES: COc1c(NC(=O)c2ccc(NC(=O)c3ccc(NC(=O)[C@H](CC#N)NC(=O)c4ccc(NC(=O)/C(C)=C/c5ccc(O)cc5)c(OC)c4O)cc3)c(OC)c2O)ccc(C(=O)O)c1O]

or a racemate, an enantiomer, a diastereomer, a pharmaceutically acceptable salt, or a derivative thereof.

In one embodiment, the compound represented by Formula (I) is a compound represented by Formula (VIIIa)

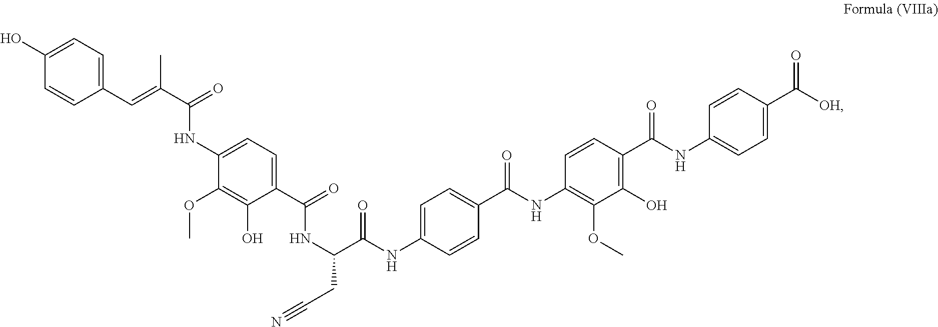 [SMILES: COc1c(NC(=O)/C(C)=C/c2ccc(O)cc2)ccc(C(=O)N[C@@H](CC#N)C(=O)Nc2ccc(C(=O)Nc3ccc(C(=O)Nc4ccc(C(=O)O)cc4)c(O)c3OC)cc2)c1O]

or a racemate, an enantiomer, a diastereomer, a pharmaceutically acceptable salt, or a derivative thereof.

In one embodiment, the compound represented by Formula (I) is a compound represented by Formula (IXa)

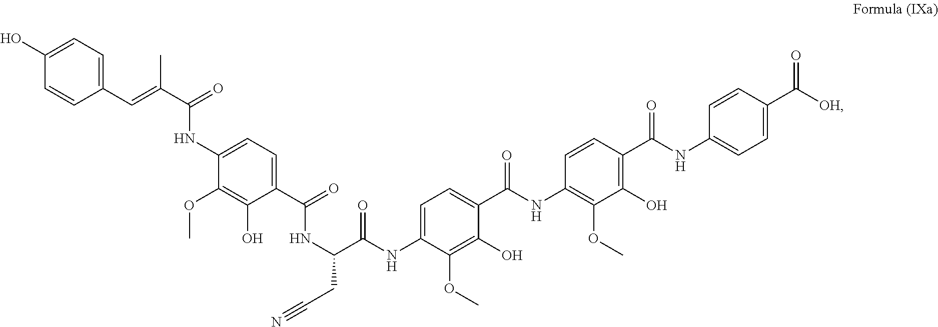 [SMILES: COc1c(NC(=O)/C(C)=C/c2ccc(O)cc2)ccc(C(=O)N[C@@H](CC#N)C(=O)Nc2ccc(C(=O)Nc3ccc(C(=O)Nc4ccc(C(=O)O)cc4)c(O)c3OC)c(O)c2OC)c1O]

or a racemate, an enantiomer, a diastereomer, a pharmaceutically acceptable salt, or a derivative thereof.

The compounds described herein may form salts with acids or bases, and such salts are included in the present invention. The term “salts” embraces addition salts of free acids or free bases that are compounds of the invention.

In one aspect, the present invention relates, in part, to compositions comprising one or more compounds of the present invention. In some embodiments, the composition comprises one or more compounds having the structure of Formulae (I)-(IX), or a racemate, an enantiomer, a diastereomer, a pharmaceutically acceptable salt, or a derivative thereof. In some embodiments, the composition is the pharmaceutical composition.

### Nucleic Acids Encoding Compounds and Methods of Generating Compounds

In one aspect, the present invention relates, in part, to a method of generating one or more compounds of the present invention. In various embodiments, the compounds of the present invention can be generated using any method known to those of skill in the art. For example, in one embodiment, the compounds can be synthesized using any method known to those of skill in the art. For example, the compounds of the present invention may be synthesized using techniques well-known in the art of organic synthesis. The starting materials and intermediates required for the synthesis may be obtained from commercial sources or synthesized according to methods known to those skilled in the art.

Examples of starting materials and intermediates required for the synthesis of one or more compounds of the present invention include, but are not limited to, compounds represented by Formulae (A)-(F)

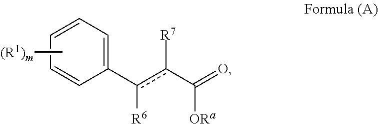

or a racemate, an enantiomer, a diastereomer, a pharmaceutically acceptable salt, or a derivative thereof,

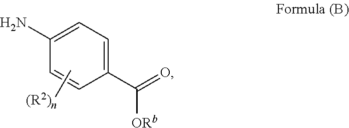

or a racemate, an enantiomer, a diastereomer, a pharmaceutically acceptable salt, or a derivative thereof,

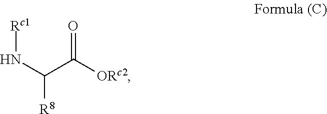

or a racemate, an enantiomer, a diastereomer, a pharmaceutically acceptable salt, or a derivative thereof,

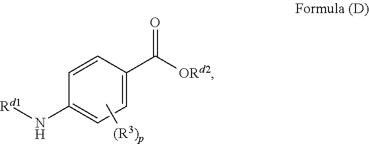

or a racemate, an enantiomer, a diastereomer, a pharmaceutically acceptable salt, or a derivative thereof,

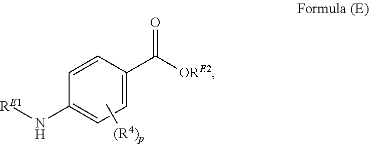

or a racemate, an enantiomer, a diastereomer, a pharmaceutically acceptable salt, or a derivative thereof, or

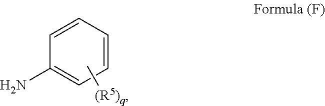

or a racemate, an enantiomer, a diastereomer, a pharmaceutically acceptable salt, or a derivative thereof.

In some embodiments, R^{a} is hydrogen, deuterium, halogen, alkyl, allyl, protecting group, an amino acid, or any combinations thereof.

In some embodiments, R^{b} is hydrogen, deuterium, halogen, alkyl, allyl, protecting group, an amino acid, or any combinations thereof.

In some embodiments, R^{c1} is hydrogen, deuterium, halogen, alkyl, allyl, protecting group, an amino acid, or any combinations thereof.

In some embodiments, R^{c2} is hydrogen, deuterium, halogen, alkyl, allyl, protecting group, an amino acid, or any combinations thereof.

In some embodiments, R^{d1} is hydrogen, deuterium, halogen, alkyl, allyl, protecting group, an amino acid, or any combinations thereof.

In some embodiments, R^{d2} is hydrogen, deuterium, halogen, alkyl, allyl, protecting group, an amino acid, or any combinations thereof.

In some embodiments, R^{e1} is hydrogen, deuterium, halogen, alkyl, allyl, protecting group, an amino acid, or any combinations thereof.

In some embodiments, R^{e2} is hydrogen, deuterium, halogen, alkyl, allyl, protecting group, an amino acid, or any combinations thereof.

In some embodiments, the protecting group is any protecting group known in the art of organic synthesis. The protecting group may be obtained from commercial sources or synthesized according to methods known to those skilled in the art. It is well understood that protecting groups for sensitive or reactive groups may be employed where necessary, in accordance with general principles of chemistry. Protecting groups are manipulated according to standard methods of organic synthesis (T. W. Greene and P. G. M. Wuts (1999) Protective Groups in Organic Synthesis, 3rd Ed., John Wiley & Sons). These groups may be removed at a convenient stage of the compound synthesis using methods that are readily apparent to those skilled in the art.

Alternatively, the compounds can be biosynthesized via heterologous expression of a biosynthetic gene. For example, in one embodiment, the compounds can be biosynthesized via heterologous expression of a biosynthetic gene.

Thus, in one aspect, the invention provides methods of biosynthesizing one or more compounds of the present invention. In one embodiment, the method comprises generating a metagenome-derived congener biosynthetic gene cluster (BGC) comprising a nucleic acid molecule, wherein the nucleic acid molecule encodes the gram-negative active compound, providing the nucleic acid compound to a host, incubating the host in a growth medium, and isolating the gram-negative active compound from the host or the growth medium. In one embodiment, the method comprises providing a heterologous nucleic acid of the invention to a host, incubating the host in a growth medium, and isolating one or more compounds of the present invention from the host or the growth medium.

In one embodiment, the one or more compounds of the present invention is isolated from the growth medium. In one embodiment, providing a heterologous nucleic acid to the host comprises transforming the host with the heterologous nucleic acid. In one embodiment, the heterologous nucleic acid comprises a sequence as set forth in SEQ ID NO: 1, or a variant or fragment thereof.

The term “heterologous nucleic acid” as used herein refers to a nucleic acid sequence, which has been introduced into the host organism, wherein said host does not endogenously comprise said nucleic acid. For example, said heterologous nucleic acid may be introduced into the host organism by recombinant methods. Thus, the genome of the host organism has been augmented by at least one incorporated heterologous nucleic acid sequence. It will be appreciated that typically the genome of a recombinant host described herein is augmented through the stable introduction of one or more heterologous nucleic acids encoding one or more compounds of the present invention.

In one embodiment, the present invention provides methods of generating one or more compounds of the present invention via isolated nucleic acids and vectors encoding one or more compounds of the present invention. Thus, in various embodiments, the present invention provides isolated nucleic acids and vectors encoding one or more compounds of the present invention. In one embodiment, when the nucleic acids and vectors are administered to a subject, they produce one or more compounds of the present invention. In one embodiment, when the nucleic acids and vectors are administered to a subject, they produce an antibacterial effect.

In one embodiment, the nucleic acid comprises a sequence as set forth in SEQ ID NO:1 or a variant or fragment thereof.

The nucleic acid sequences include both the DNA sequence that is transcribed into RNA and the RNA sequence that is translated into a polypeptide. According to other embodiments, the polynucleotides of the invention are inferred from the amino acid sequence of the polypeptides of the invention. As is known in the art several alternative polynucleotides are possible due to redundant codons, while retaining the biological activity of the translated polypeptides.

It is to be understood explicitly that the scope of the present invention encompasses homologs, analogs, variants, fragments, derivatives and salts, including shorter and longer polynucleotides as well as polynucleotide analogs with one or more nucleic acid substitution, as well as nucleic acid derivatives, non-natural nucleic acids and synthetic nucleic acids as are known in the art, with the stipulation that these modifications must preserve the activity of the original molecule. The invention should be construed to include any and all isolated nucleic acids which are homologous to the nucleic acids described and referenced herein.

The skilled artisan would understand that the nucleic acids of the invention encompass a RNA or a DNA sequence comprising a sequence of the invention, and any modified forms thereof, including chemical modifications of the DNA or RNA which render the nucleotide sequence more stable when it is cell free or when it is associated with a cell. Chemical modifications of nucleotides may also be used to enhance the efficiency with which a nucleotide sequence is taken up by a cell or the efficiency with which it is expressed in a cell. Any and all combinations of modifications of the nucleotide sequences are contemplated in the present invention.

Vectors include, but are not limited to, plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

In one embodiment, the vector is a plasmid. The plasmid may comprise one or more sequences encoding one or more compounds described herein. The plasmid may further comprise an initiation codon, which may be upstream of the coding sequence, and a stop codon, which may be downstream of the coding sequence. The initiation and termination codon may be in frame with the coding sequence.

The plasmid may also comprise a promoter that is operably linked to the coding sequence The promoter operably linked to the coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, or human metalothionein. The promoter may also be a tissue specific promoter, such as a muscle or skin specific promoter, natural or synthetic. Examples of such promoters are described in US patent application publication no. US20040175727, the contents of which are incorporated herein in its entirety.

The plasmid may also comprise a polyadenylation signal, which may be downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA).

The plasmid may also comprise an enhancer upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may also comprise a regulatory sequence, which may be well suited for gene expression in a cell into which the plasmid is administered. The coding sequence may comprise a codon that may allow more efficient transcription of the coding sequence in the host cell. In one embodiment, the plasmid may be pTARa (Invitrogen, San Diego, Calif) plasmid.

Also provided herein is a linear nucleic acid vaccine, or linear expression cassette (“LEC”). The LEC may be any linear DNA devoid of any phosphate backbone. The DNA may encode one or more compounds of the present invention. The LEC may contain a promoter, an intron, a stop codon, a polyadenylation signal. The expression of the antigen may be controlled by the promoter. The LEC may not contain any antibiotic resistance genes and/or a phosphate backbone. The LEC may not contain other nucleic acid sequences unrelated to the desired compound expression.

The LEC may be derived from any plasmid capable of being linearized. The plasmid may be capable of expressing one or more compounds of the present invention. The plasmid may be any expression vector capable of expressing the DNA.

In one embodiment, viral vectors are provided herein which are capable of delivering a nucleic acid of the invention to a cell. The expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Suitable host organisms include microorganisms, plant cells, and plants. The microorganism can be any microorganism suitable for expression of heterologous nucleic acids. In one embodiment the host organism of the invention is a eukaryotic cell. In another embodiment the host organism is a prokaryotic cell. In one embodiment, the host organism is a fungal cell such as a yeast or filamentous fungus. In one embodiment the host organism may be a yeast cell.

The host organism may also be a plant, plant or plant cell can be transformed by having a heterologous nucleic acid integrated into its genome, i.e., it can be stably transformed. Stably transformed cells typically retain the introduced nucleic acid with each cell division. A plant or plant cell can also be transiently transformed such that the recombinant gene is not integrated into its genome. Transiently transformed cells typically lose all or some portion of the introduced nucleic acid with each cell division such that the introduced nucleic acid cannot be detected in daughter cells after a certain number of cell divisions.

In one aspect, the present invention provides an engineered cell that expresses one or more compounds of the present invention. The genetically modified cell according to the invention may be constructed from any suitable host cell. The host cell may be an unmodified cell or may already be genetically modified. The cell may be a prokaryote cell, a eukaryote cell, a plant cell or an animal cell.

In one embodiment, the engineered cell is modified by way of introducing genetic material into the cell in order for the cell to produce one or more compounds of the present invention. In one embodiment, the engineered cell is modified by way of transforming a nucleic acid of the invention into the cell.

In one embodiment, the engineered cell produces a compound of Formula (I). In some embodiments, the engineered cell produces at least one compound of Formula (I)-(IX). For example, in one embodiment, the engineered cell produces a compound of Formula (VIII). In one embodiment, the engineered cell produces a compound of Formula (IX).

In one embodiment, the engineered cell produces a compound of Formula (I′). In one embodiment, the engineered cell produces a compound of Formula (I″). In some embodiments, the engineered cell produces at least one compound of Formula (IIa)-(IXa). For example, in one embodiment, the engineered cell produces a compound of Formula (VIIIa). In one embodiment, the engineered cell produces a compound of Formula (IXa).

In one embodiment, the cell is a eukaryotic cell. In one embodiment, the cell may be a human cell, a non-human mammalian cell, a non-mammalian vertebrate cell, an invertebrate cell, an insect cell, a plant cell, a yeast cell, or a single cell eukaryotic organism. In one embodiment, the cell may be an adult cell or an embryonic cell (e.g., an embryo). In one embodiment, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, unipotent stem cells and others.

In one embodiment, the cell is a cell line cell. Non-limiting examples of suitable mammalian cells include Chinese hamster ovary (CHO) cells, baby hamster kidney (BHK) cells; mouse myeloma NS0 cells, mouse embryonic fibroblast 3T3 cells (NIH3T3), mouse B lymphoma A20 cells; mouse melanoma B16 cells; mouse myoblast C2C12 cells; mouse myeloma SP2/0 cells; mouse embryonic mesenchymal C3H-10T½ cells; mouse carcinoma CT26 cells, mouse prostate DuCuP cells; mouse breast EMT6 cells; mouse hepatoma Hepa1c1c7 cells; mouse myeloma J5582 cells; mouse epithelial MTD-1A cells; mouse myocardial MyEnd cells; mouse renal RenCa cells; mouse pancreatic RIN-5F cells; mouse melanoma ×64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; African green monkey kidney (VERO-76) cells; human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, human SW48 cells, human HCT116 cells, and human K562 cells. An extensive list of mammalian cell lines may be found in the American Type Culture Collection catalog (ATCC, Manassas, Va.).

In one embodiment, the cell can be a prokaryotic cell or a eukaryotic cell. In one embodiment, the cell is a prokaryotic cell. In one embodiment, the cell is a genetically engineered bacteria cell.

In one embodiment, the genetically engineered bacteria cell is a non-pathogenic bacteria cell. In some embodiments, the genetically engineered bacteria cell is a commensal bacteria cell. In some embodiments, the genetically engineered bacteria cell is a probiotic bacteria cell. In some embodiments, the genetically engineered bacteria cell is a naturally pathogenic bacteria cell that is modified or mutated to reduce or eliminate pathogenicity. Exemplary bacteria include, but are not limited to Streptomyces albus, Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii.

In one embodiment, the host is a Streptomyces albus cell.

In some embodiments, the genetically engineered bacteria are Escherichia coli strain Nissle 1917 (E. coli Nissle), a Gram-negative bacterium of the Enterobacteriaceae family that “has evolved into one of the best characterized probiotics” (Ukena et al., 2007). The strain is characterized by its complete harmlessness (Schultz, 2008), and has GRAS (generally recognized as safe) status (Reister et al., 2014, emphasis added). Genomic sequencing confirmed that E. coli Nissle lacks prominent virulence factors (e.g., E. coli α-hemolysin, P-fimbrial adhesins) (Schultz, 2008). In addition, it has been shown that E. coli Nissle does not carry pathogenic adhesion factors, does not produce any enterotoxins or cytotoxins, is not invasive, and not uropathogenic (Sonnenborn et al., 2009). As early as in 1917, E. coli Nissle was packaged into medicinal capsules, called Mutaflor, for therapeutic use. E. coli Nissle has since been used to treat ulcerative colitis in humans in vivo (Rembacken et al., 1999), to treat inflammatory bowel disease, Crohn's disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle's therapeutic efficacy and safety have convincingly been proven (Ukena et al., 2007).

One of ordinary skill in the art would appreciate that the genetic modifications disclosed herein may be modified and adapted for other species, strains, and subtypes of bacteria.

### Treatment Methods

In one aspect, the invention provides methods of treating or preventing an infection in a subject in need thereof. In some embodiments, the method comprises administering to the subject an effective amount of a composition comprising at least one compound of the invention (e.g., at least one compound of Formulae (I)-(IX)). In some embodiments, the method comprises administering to the subject an effective amount of a composition comprising at least one nucleic acid of the invention.

In some embodiments, the method treats or prevents a bacterial infection. In one embodiment, the method treats or prevents a gram-positive bacterial infection. In one embodiment, the bacterial infection is resistant to antibiotics. For example, in one embodiment, the bacterial infection is resistant to one or more of, beta-lactams, including methicillin, oxacillin, or penicillin, tetracyclines, gentamicin, kanamycin, erythromycin, spectinomycin, and vancomycin.

Exemplary bacterial infections that may be treated by way of the present invention includes, but is not limited to, infections caused by bacteria from the taxonomic genus of Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, and Yersinia. In some embodiments, the bacterial infection is an infection of Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Klebsiella species, Legionella pneumophila, Leptospira interrogans, Leptospira santarosai, Leptospira welii, Leptospira noguchii, Listeria monocytogenes, Morexella species, Mycobacterium leprae, Mycobacterium tuberculosis, Mycobacterium ulcerans, Mycoplasma pneumoniae, Neisseria gonorrhoeae, Neisseria meningitidis, Proteus species, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Ireponema pallidum, Ureaplasma urealyticum, Vibrio cholerae, Yersinia pestis, Yersinia enterocolitica, or Yersinia pseudotuberculosis. In one embodiment, the bacterial infection is a Listeria monocytogenes infection.

In one embodiment, the bacterial infection is an infection of S. aureus USA300, S. aureus COL, S. aureus BAA-42, S. aureus NRS100, S. aureus NRS108, S. aureus NRS140, S. aureus NRS146, E. faecium VRE, E. faecium Com15, S. pneumoniae, S. mutans, B. subtilis, L. rhamnosus, E. coli, C. albicans, or C. neoformans.

Exemplary diseases caused by bacterial infections which may be treated using compositions of the present invention, include but are not limited to, bacterially mediated meningitis, sinus tract infections, pneumonia, endocarditis, pancreatitis, appendicitis, gastroenteritis, biliary tract infections, soft tissue infections, urinary tract infections, cystitis, pyelonephritis, osteomyelitis, bacteremia, Actinomycosis, Whooping cough, Secondary bacterial pneumonia, Lyme disease (B. burgdorferi), Relapsing fever, Brucellosis, Enteritis, bloody diarrhea, Guillain-Barre syndrome, Atypical pneumonia, Trachoma, Neonatal conjunctivitis, Neonatal pneumonia, Nongonococcal urethritis(NGU), Urethritis, Pelvic inflammatory disease, Epididymitis, Prostatitis, Lymphogranuloma venereum (LGV), Psittacosis, Botulism: Mainly muscle weakness and paralysis, Pseudomembranous colitis, Anaerobic cellulitis, Gas gangrene Acutefood poisoning, Tetanus, and Diphtheria.

However, the invention should not be limited to only treating bacterial infection. The invention encompasses compounds having an antimicrobial activity including but not limited to antibacterial, antimycobacterial, antifungal, antiviral and the likes.

In one aspect, the invention provides methods of killing a bacterial cell or inhibiting the grown of a bacterial cell. In some embodiments, the method comprises administering to the cell an effective amount of a composition comprising at least one compound of the invention. In some embodiments, the method comprises administering to the cell an effective amount of a composition comprising at least one nucleic acid of the invention. In one embodiment the bacterial cell is a gram positive bacterial cell. In one embodiment, the bacterial cell is resistant to antibiotics. For example, in one embodiment, the bacterial cell is resistant to one or more of, beta-lactams, including methicillin, oxacillin, or penicillin, tetracyclines, gentamicin, kanamycin, erythromycin, spectinomycin, and vancomycin.

In another aspect, the invention provides compositions and methods for treating and/or preventing a disease or disorder related to the detrimental growth and/or proliferation of a bacterial cell in vivo, ex vivo or in vitro. In certain embodiments, the method comprises administering a composition comprising an effective amount of a composition provided by the invention to a subject, wherein the composition is effective in inhibiting or preventing the growth and/or proliferation of a bacterial cell. In certain embodiments, the bacterial cell is a Gram-positive bacterial cell, e.g., a bacteria of a genera such as Staphylococcus, Streptococcus, Enterococcus, (which are cocci) and Bacillus, Corynebacterium, Nocardia, Clostridium, Actinobacteria, and Listeria (which are rods and can be remembered by the mnemonic obconical), Mollicutes, bacteria-like Mycoplasma, Actinobacteria.

In certain embodiments, the bacterial cell is a Gram-bacteria cell, e.g., a bacteria of a genera such as Citrobacter, Yersinia, Pseudomonas and Escherichia, Hemophilus, Neisseria, Klebsiella, Legionella, Helicobacter, and Salmonella. The compounds as described herein and compositions comprising them may thus be for use in the treatment of bacterial infections by the above-mentioned Gram+ or Gram− bacteria.

In one embodiment, the method further comprises administering a second therapeutic agent. In one embodiment, the second therapeutic agent is an antibiotic agent. In one embodiment, the compound of the invention and the at least one additional antibiotic agent act synergistically in preventing, reducing or disrupting microbial growth.

Non-limiting examples of the at least one additional antibiotic agents include levofloxacin, doxycycline, neomycin, clindamycin, minocycline, gentamycin, rifampin, chlorhexidine, chloroxylenol, methylisothizolone, thymol, α-terpineol, cetylpyridinium chloride, hexachlorophene, triclosan, nitrofurantoin, erythromycin, nafcillin, cefazolin, imipenem, astreonam, gentamicin, sulfamethoxazole, vancomycin, ciprofloxacin, trimethoprim, rifampin, metronidazole, clindamycin, teicoplanin, mupirocin, azithromycin, clarithromycin, ofoxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linexolid, temafloxacin, tosufloxacin, clinafloxacin, sulbactam, clavulanic acid, amphotericin B, fluconazole, itraconazole, ketoconazole, nystatin, penicillins, cephalosporins, carbepenems, beta-lactams antibiotics, aminoglycosides, macrolides, lincosamides, glycopeptides, tetracylines, chloramphenicol, quinolones, fucidines, sulfonamides, trimethoprims, rifamycins, oxalines, streptogramins, lipopeptides, ketolides, polyenes, azoles, echinocandines, and any combination thereof.

In one embodiment, the compositions of the invention find use in removing at least a portion of or reducing the number of microorganisms and/or biofilm-embedded microorganisms attached to the surface of a medical device or the surface of a subject's body (such as the skin of the subject, or a mucous membrane of the subject, such as the vagina, anus, throat, eyes or ears). In one embodiment, the compositions of the invention find further use in coating the surface of a medical device, thus inhibiting or disrupting microbial growth and/or inhibiting or disrupting the formation of biofilm on the surface of the medical device. The compositions of the invention find further use in preventing or reducing the growth or proliferation of microorganisms and/or biofilm-embedded microorganisms on the surface of a medical device or on the surface of a subject's body. However, the invention is not limited to applications in the medical field. Rather, the invention includes using one or more compounds of the present invention or an analog thereof as an antimicrobial and/or antibiofilm agent in any setting.

The composition of the invention may be administered to a patient or subject in need in a wide variety of ways, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the composition is administered systemically to the subject. In one embodiment, the compositions of the present invention are administered to a patient by i.v. injection. In one embodiment, the composition is administered locally to the subject. In one embodiment, the compositions of the present invention are administered to a patient topically. Any administration may be a single application of a composition of invention or multiple applications. Administrations may be to single site or to more than one site in the individual to be treated. Multiple administrations may occur essentially at the same time or separated in time.

In one aspect, the compositions of the invention may be in the form of a coating that is applied to the surface of a medical device or the surface of a subject's body. In one embodiment, the coating prevents or hinders microorganisms and/or biofilm-embedded microorganisms from growing and proliferating on at least one surface of the medical device or at least one surface of the subject's body. In another embodiment, the coating facilitates access of antimicrobial agents to the microorganisms and/or biofilm-embedded microorganisms, thus helping prevent or hinder the microorganisms and/or biofilm-embedded microorganisms from growing or proliferating on at least one surface of the medical device or at least one surface of the subject's body. The compositions of the invention may also be in the form of a liquid or solution, used to clean the surface of medical device or the surface of a subject's body, on which microorganisms and/or biofilm-embedded microorganisms live and proliferate. Such cleaning of the medical device or body surface may occur by flushing, rinsing, soaking, or any additional cleaning method known to those skilled in the art, thus removing at least a portion of or reducing the number of microorganisms and/or biofilm-embedded microorganisms attached to at least one surface of the medical device or at least one surface of the subject's body.

Subjects to which administration of the pharmaceutical compositions of the invention is contemplated include, but are not limited to, humans and other primates, mammals including but not limited to non-human mammals such as non-human primates, cattle, pigs, horses, sheep, cats, and dogs.

Pharmaceutical compositions of the present invention may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present invention to be administered can be determined by a physician with consideration of individual differences in age, weight, disease type, extent of disease, and condition of the patient (subject).

### Dosage and Formulation (Pharmaceutical Compositions)

The invention also encompasses the use of pharmaceutical compositions comprising a compound of the invention, a nucleic acid of the invention, or salts thereof. Such a pharmaceutical composition may comprise of at least one a compound of the invention, a nucleic acid of the invention, or salts thereof in a form suitable for administration to a subject, or the pharmaceutical composition may comprise at least one a compound of the invention, a nucleic acid of the invention, or salts thereof, and one or more pharmaceutically acceptable carriers, one or more additional ingredients, or some combination of these. The compound or nucleic acid of the invention may be present in the pharmaceutical composition in the form of a physiologically acceptable salt, such as in combination with a physiologically acceptable cation or anion, as is well known in the art.

Administration of the therapeutic agent in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the subject, and whether prevention or treatment is to be achieved. Such factors can be readily determined by the clinician employing animal models or other test systems which are well known to the art

The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

In one embodiment, the pharmaceutical compositions useful for practicing the methods of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

Typically, dosages which may be administered in a method of the invention to a mammal, preferably a human, range in amount from 0.5 g to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 1 μg to about 10 mg per kilogram of body weight of the mammal. More preferably, the dosage will vary from about 3 g to about 5 mg per kilogram of body weight of the mammal.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The compounds and polypeptides (active ingredients) of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the pharmaceutical composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

The present invention also provides pharmaceutical compositions comprising one or more of the compositions described herein. Formulations may be employed in admixtures with conventional excipients, i.e., pharmaceutically acceptable organic or inorganic carrier substances suitable for administration to subject. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

In an embodiment, the composition includes an anti-oxidant and a chelating agent that inhibits the degradation of one or more components of the composition. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the HMW-HA or other composition of the invention in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments there between.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

Routes of administration of any of the compositions of the invention include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

These methods described herein are by no means all-inclusive, and further methods to suit the specific application will be apparent to the ordinary skilled artisan. Moreover, the effective amount of the compositions can be further approximated through analogy to compounds known to exert the desired effect.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Metagenome Guided Medicinal Chemistry Yielded Improved Gram-Negative Active Albicidin and Cystobactamid Type Antibiotics

In almost all cases, a characterized natural product represents only one example of a larger family of naturally occurring structures. These natural congeners arise as a result of selective pressure to have different potencies, responses to resistance, and spectra of activity. Soil metagenomes contain large collections of bacteria, including many difficult-to-culture species making them potentially rich sources of congener biosynthetic gene clusters (BGCs). Bioinformatic analysis of BGCs that encode congeners of an antibiotic of interest can serve as valuable guides for medicinal chemistry efforts designed to improve natural product lead structures. Unfortunately, as sequencing of the global microbiome is still in its infancy, many families of natural products, especially those that arise from previously underexplored taxa, have few congener BGCs deposited in publicly available databases.

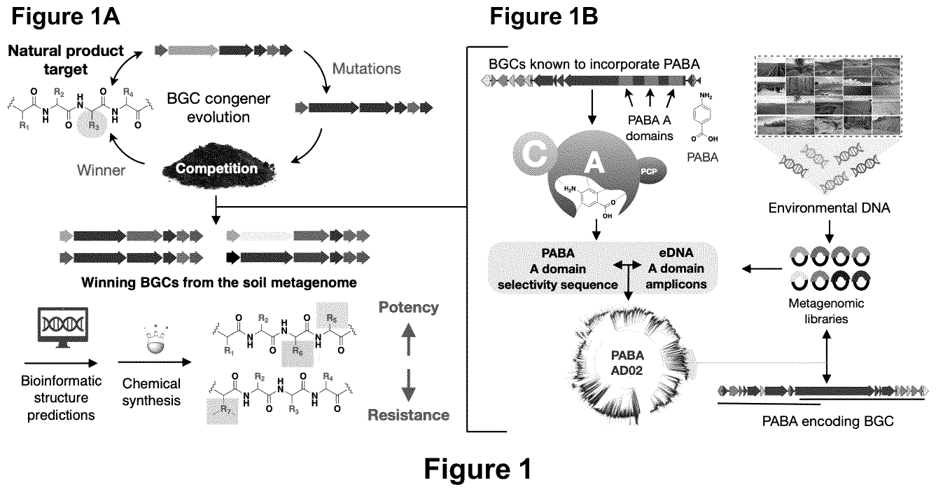

The present studies investigated combining soil metagenomics with bioinformatic natural product structure generation methods to intelligently guide the synthesis of albicidin and cystobactamid analogs with improved antibacterial activity (i.e., potency and spectrum) and the ability to overcome naturally occurring resistance, two likely outcomes of diverse selective pressures that drive the evolution of natural antibiotic congeners (FIG. 1A). Albicidin and cystobactamid are closely related ρ-amino benzoic acid (PABA)-containing antibiotics that target DNA gyrase (Baumann S et al., 2014, Angew Chem Int Ed Engl, 53:14605-14609; Cociancich S et al., 2015, Nat Chem Biol, 11:195-197). Their potent Gram-negative activity and unique chemical structures make them appealing leads for the development of novel therapeutics (Cociancich S et al., 2015, Nat Chem Biol, 2015, 11:195-197, Hashimi S M et al., 2007, Antimicrob Agents Chemother, 51:181-187).

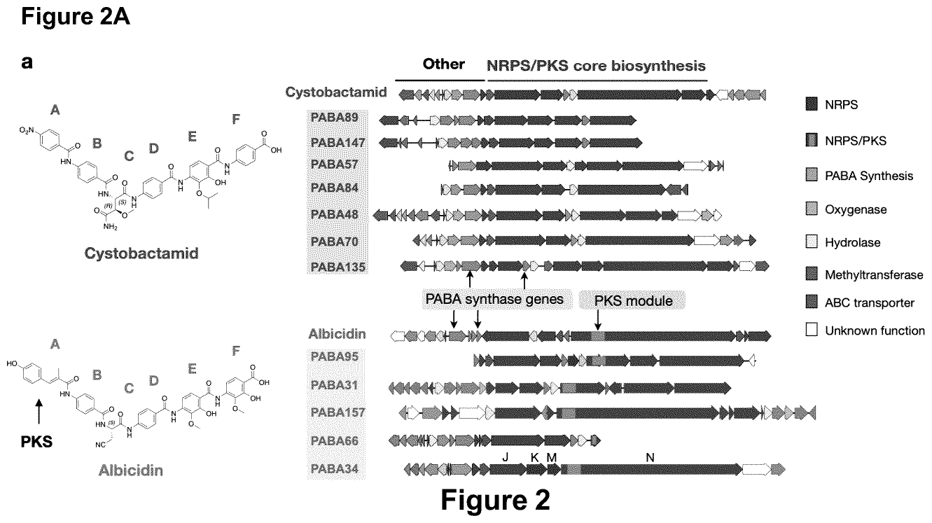

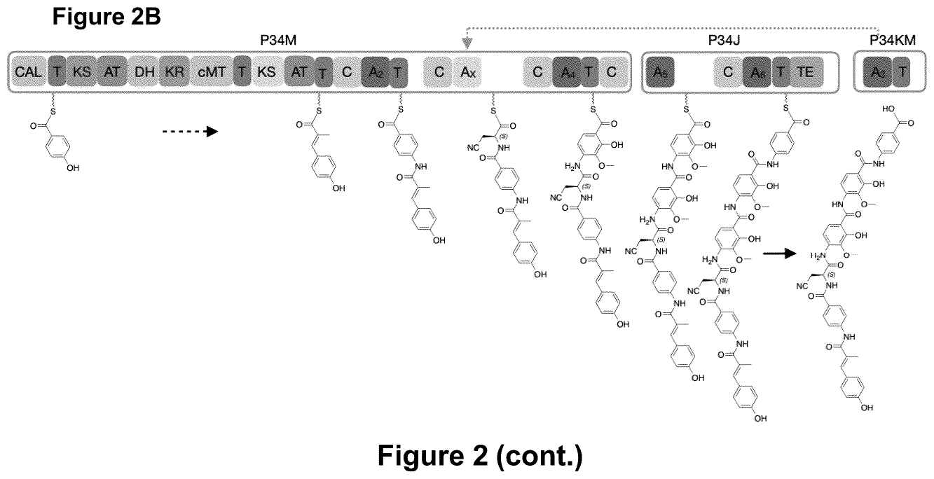

However, the identification of an endopeptidase (AlbD) that conferred resistance by antibiotic cleavage indicated that this resistance eventually threatens their therapeutic utility (Vieweg L et al., 2015, J Am Chem Soc, 137, 7608-7611; Planke T et al., 2020, Chemistry, 26:4289-4296). The present studies searched for PABA specific adenylation domain sequences to identify albicidin and cystobactamid BGCs in soil metagenomes. Although they were originally isolated from traditionally underexplored bacterial taxa (Xanthomonas albilineans and Cystobacter spp, respectively) (Baumann S et al., 2014, Angew Chem Int Ed Engl, 53:14605-14609; Birch R et al., 1983, Phytopathology, 73:1368-1374), the present studies found that congener BGCs were common in soil metagenomes and that natural structural variations encoded by these BGCs were different from those explored in previous medicinal chemistry studies (FIG. 2A). Synthesis of albicidin and cystobactamid analogs generated from metagenomic BGCs (i.e., synthetic bioinformatic natural products or syn-BNPs) (Chu J et al., 2016, Nat Chem Biol, 12:1004-1006) led to the herein-described identification of antibiotics with improved potency as well as the ability to circumvent AlbD cleavage without a loss of antibiosis. The herein presented general approach of using congener BGCs to guide medicinal chemistry studies represents a broadly applicable method for identifying natural products with improved biomedically relevant properties.

To identify albicidin and cystobactamid congener BGCs, the present studies screened previously archived metagenomic libraries for PABA-specific adenylation (A) domain sequences. In total, these libraries contain ˜720 million unique cosmid clones. Their construction and arraying have been described previously (Brady S F et al., 2007, Nat Protoc, 2:1297-1305; Chang F Y et al., 2015, J Am Chem Soc, 137:6044-6052; Kang H S et al., 2013, Angew Chem Int Ed Engl, 52:11063-11067). Briefly, environmental DNA (eDNA) extracted directly from diverse soils was cloned into a cosmid vector and then introduced into E. coli using lambda phage (Owen J G et al., 2013, Proc Natl Acad Sci USA, 110:11797-11802; Owen J G et al., 2015, Proc Natl Acad Sci USA, 112:4221-4226). To facilitate the recovery of specific BGCs of interest, each library of >2×10⁷ cosmid clones was arrayed into collections of subpools each containing ˜25,000 unique cosmid clones. Library subpools were screened using subpool-specific barcoded A domain degenerate primers. PCR amplicons were then sequenced using Illumina MiSeq technology and the resulting reads from each library subpool were clustered at 95% identity to generate natural product sequence tags (NPSTs) that can be used to guide the discovery of BGCs of interest. Using our environmental surveyor of natural product diversity (eSNaPD) software package (Reddy B V et al., 2014, Chem Biol, 21:1023-1033), library-derived NPSTs were compared to PABA-specific A domain sequences from the albicidin and cystobactamid BGCs. NPSTs that returned eValues of <10⁻²⁵ were considered potential PABA-specific A domains and used to construct a PABA A domain phylogenetic tree (FIG. 1B). NPSTs that grouped most closely with PABA-specific A domains from albicidin and cystobactamid biosynthesis were considered markers for congener BGCs. Sets of overlapping cosmids associated with generated PABA NPSTs were isolated from the appropriate library subpools and sequenced to reveal twelve complete and partial cystobactamid or albicidin-like BGCs (FIG. 2).

The structure of the natural product encoded by each metagenomic BGC was generated based on the presence or absence of a PKS module, the types of PABA modification enzymes it encodes, and most importantly the bioinformatically-derived substrate specificity of each NRPS A domain. For this analysis, ten conserved amino acids in the A domain substrate binding pocket were used to determine the substrate specificity (positions 235, 236, 239, 278, 299, 301, 322, 330, 331 and 517) (Stachelhaus T et al., 1999, Chem Biol, 6:493-505). The present studies compared the A domain substrate binding pockets found in each metagenomic NRPS with characterized A domain substrate binding pockets (Table 1 through Table 8).

TABLE 1
Albicidin 10 signature code analysis.

| | A domain signature | | | | | | | | | | |
|---|---|---|---|---|---|---|---|---|---|---|---|
| Module | 235 | 236 | 239 | 278 | 299 | 301 | 322 | 330 | 331 | 517 | Predicted substrate |
| A | PKS | | | | | | | | | | MCA |
| B | A | V | K | Y | V | A | N | D | A | K | PABA |
| * | E | L | T | Y | V | H | V | A | A | K | inactive |
| C | D | L | T | K | I | G | E | V | G | K | Asn |
| D | A | V | K | Y | V | A | N | D | A | K | PABA |
| E | A | I | K | Y | F | S | I | D | M | K | AHIBA |
| F | A | I | K | Y | F | S | I | D | M | K | AHIBA |

TABLE 2
Cystobactamid 10 signature code analysis.

| | A domain signature | | | | | | | | | | |
|---|---|---|---|---|---|---|---|---|---|---|---|
| Module | 235 | 236 | 239 | 278 | 299 | 301 | 322 | 330 | 331 | 517 | Predicted substrate |
| A | A | V | K | H | I | A | N | D | V | K | PABA |
| B | A | V | K | H | I | A | N | D | V | K | PABA |
| * | D | L | A | Y | F | G | V | I | G | K | inactive |
| C | D | L | T | K | I | G | E | V | G | K | Asn |
| D | A | V | K | H | I | A | N | D | V | K | PABA |
| E | A | I | K | Y | Y | S | I | D | V | K | AHMBA |
| F | A | I | K | Y | I | A | N | D | I | K | PABA |

TABLE 3
PABA48 10 signature code analysis.

| | A domain signature | | | | | | | | | | |
|---|---|---|---|---|---|---|---|---|---|---|---|
| Module | 235 | 236 | 239 | 278 | 299 | 301 | 322 | 330 | 331 | 517 | Predicted substrate |
| A | D | A | W | T | I | A | A | V | C | K | Phe |
| B | A | V | K | H | I | A | N | D | V | K | PABA |
| * | D | L | A | Y | F | G | V | I | G | K | inactive |
| C | D | L | T | K | I | G | E | V | G | K | Asn |
| D | A | V | K | H | I | A | N | D | V | K | PABA |
| E | A | I | K | Y | F | S | I | D | V | K | AHIBA |
| F | A | I | K | Y | I | A | N | D | I | K | AHIBA |

TABLE 4
PABA70 10 signature code analysis.

| | A domain signature | | | | | | | | | | |
|---|---|---|---|---|---|---|---|---|---|---|---|
| Module | 235 | 236 | 239 | 278 | 299 | 301 | 322 | 330 | 331 | 517 | Predicted substrate |
| A | D | A | A | T | I | A | A | V | C | K | Tyr |
| B | A | V | K | Y | V | A | N | D | V | K | PABA |
| * | E | L | A | Y | F | G | I | I | G | K | inactive |
| C | D | L | T | K | I | G | E | V | G | K | Asn |
| D | A | V | K | Y | V | A | N | D | V | K | PABA |
| E | A | I | K | Y | F | S | I | D | V | K | AHIBA |
| F | A | I | K | C | F | S | I | D | I | K | AHIBA |

TABLE 5
PABA57 10 signature code analysis.

| | A domain signature | | | | | | | | | | |
|---|---|---|---|---|---|---|---|---|---|---|---|
| Module | 235 | 236 | 239 | 278 | 299 | 301 | 322 | 330 | 331 | 517 | Predicted substrate |
| A | D | A | A | T | I | A | A | V | C | K | Tyr |
| B | A | V | K | H | I | A | N | D | I | K | PABA |
| * | D | L | A | H | F | G | T | I | G | K | inactive |
| C | D | L | T | K | I | G | E | V | G | K | Asn |
| D | A | V | K | H | I | A | N | D | I | K | PABA |
| E | A | I | K | Y | F | S | I | D | I | K | AHIBA |
| F | A | V | K | H | I | A | N | D | I | K | PABA |

TABLE 6
PABA34 10 signature code analysis.

| | A domain signature | | | | | | | | | | |
|---|---|---|---|---|---|---|---|---|---|---|---|
| Module | 235 | 236 | 239 | 278 | 299 | 301 | 322 | 330 | 331 | 517 | Predicted substrate |
| A | PKS | | | | | | | | | | MCA |
| B | A | V | K | H | V | A | N | D | V | K | PABA |
| * | D | I | I | I | L | A | I | E | I | K | inactive |
| C | D | L | T | K | I | G | E | V | G | K | Asn |
| D | A | I | K | Y | F | S | I | D | M | K | AHMBA |
| E | A | I | K | Y | F | S | I | D | M | K | AHMBA |
| F | A | V | K | H | V | A | N | D | V | K | PABA |

TABLE 7
PABA157 10 signature code analysis.

| | A domain signature | | | | | | | | | | |
|---|---|---|---|---|---|---|---|---|---|---|---|
| Module | 235 | 236 | 239 | 278 | 299 | 301 | 322 | 330 | 331 | 517 | Predicted substrate |
| A | PKS | | | | | | | | | | CA |
| B | A | V | K | F | V | A | N | D | V | K | PABA |
| * | G | L | L | S | E | H | V | H | F | K | inactive |
| C | D | L | T | K | I | G | E | V | G | K | Asn |
| D | A | V | K | F | V | A | N | D | V | K | AHMBA |
| E | A | I | K | Y | F | S | I | D | I | K | AHMBA |
| F | A | V | K | F | V | A | N | D | V | K | PABA |

TABLE 8
PABA95 10 signature code analysis.

| | A domain signature | | | | | | | | | | |
|---|---|---|---|---|---|---|---|---|---|---|---|
| Module | 235 | 236 | 239 | 278 | 299 | 301 | 322 | 330 | 331 | 517 | Predicted substrate |
| PKS | | | | | | | | | | | MCA |
| B | A | I | K | Y | F | S | I | D | I | K | AHMBA |
| * | D | V | I | Y | L | G | A | L | G | K | inactive |
| C | D | L | T | K | I | G | E | V | G | K | Asn |
| D | A | V | K | H | I | A | N | D | V | K | PABA |
| E | A | I | K | Y | F | S | I | D | V | K | AHMBA |
| F | A | V | K | F | V | A | N | D | V | K | PABA |

Signature sequences for PABA as well as modified PABAs (4-amino-2-hyroxy-3-isopropoxybenzoic acid (AHIBA) or 4-amino-2-hydroxy-3-methoxylbenzoic acid (AHMBA)) were derived from A domains found in the albicidin and cystobactamid BGCs (Table 1 and Table 2). An analysis of the tailoring genes that encode the differential functionalization of PABA allowed to distinguish between the presence of AHIBA and AHMBA in the generated product of each BGC.

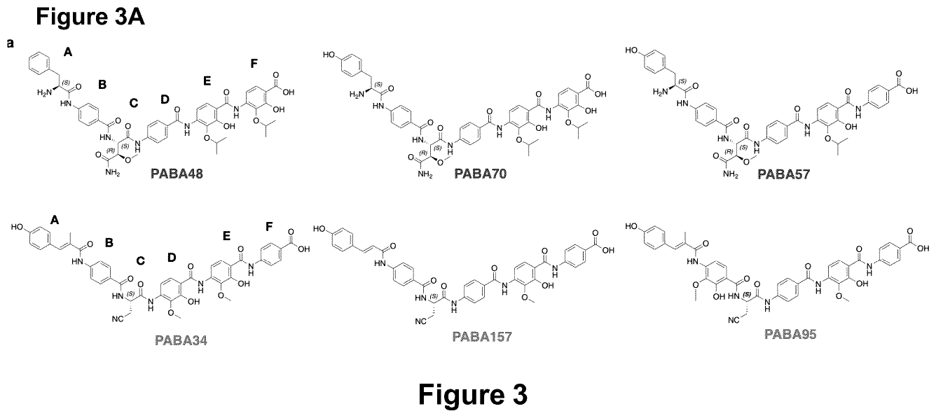

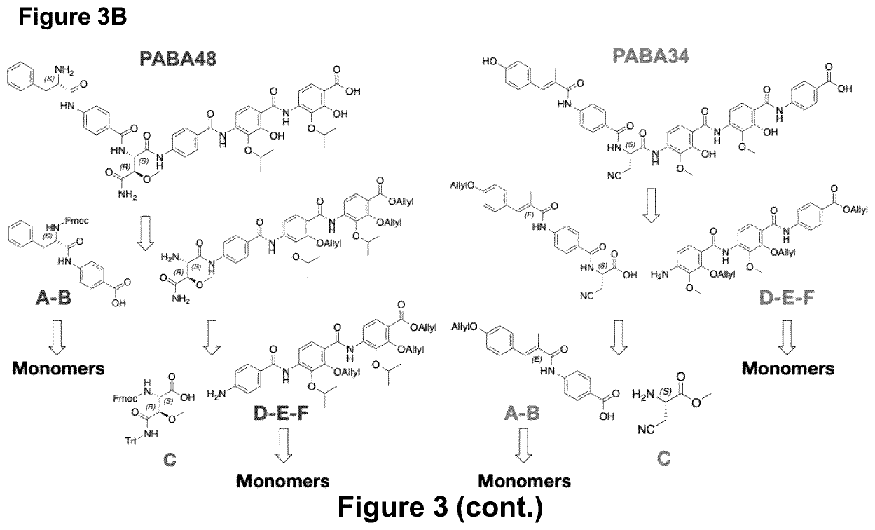

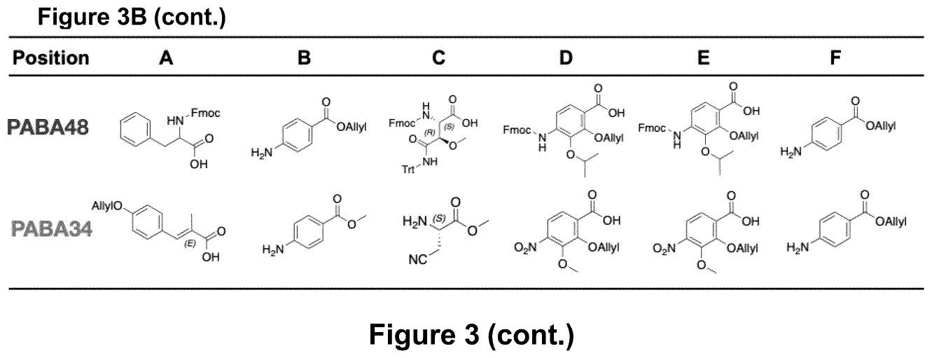

Among the seven cystobactamid-like BGCs that were identified, three different structural analogs were generated, which are embodied by BGCs PABA48, PABA70 and PABA57. Each unique bioinformatically-derived cystobactamid congener BGC encodes six NRPS modules. A domain substrate specificity analysis of the six A domains in each BGC together with an analysis of the tailoring genes in these BGCs indicated that the central four residues (positions B, C, D, E) were conserved across this family of congeners (FIG. 2A and FIG. 3A): PABA, β-methoxyasparagine (MO-Asn), PABA, and AHIBA. In place of the PABA seen at the N-terminus of known cystobactamids, the herein described analysis indicated that congener BGCs incorporate either tyrosine or phenylalanine at this position. The C-terminal residue was indicated to be an AHTBA in two cystobactamid congeners (PABA48 and PABA70) and PABA in the third (PABA57). Cystobactamid congener BGCs were indicated to encode a B12-dependent radical SAM enzyme (CysS homolog) that introduces the t-butyl functionality onto AHIBA, which was consistent with a gene cluster that encodes a metabolite containing AHIBA, (Wang Y et al., 2020, J Am Chem Soc, 142:9944-9954).

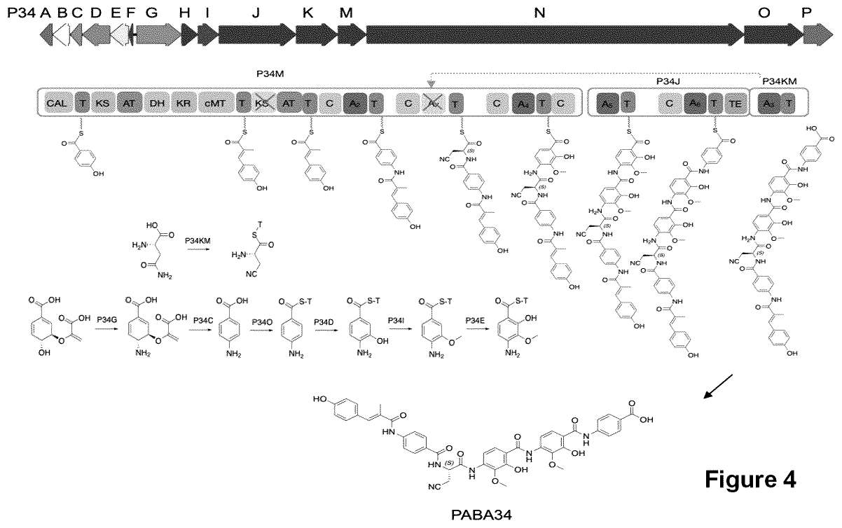

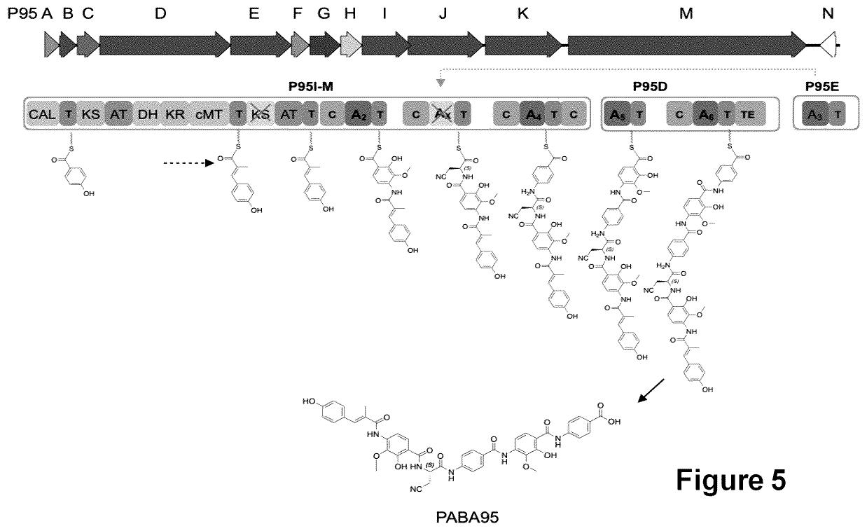

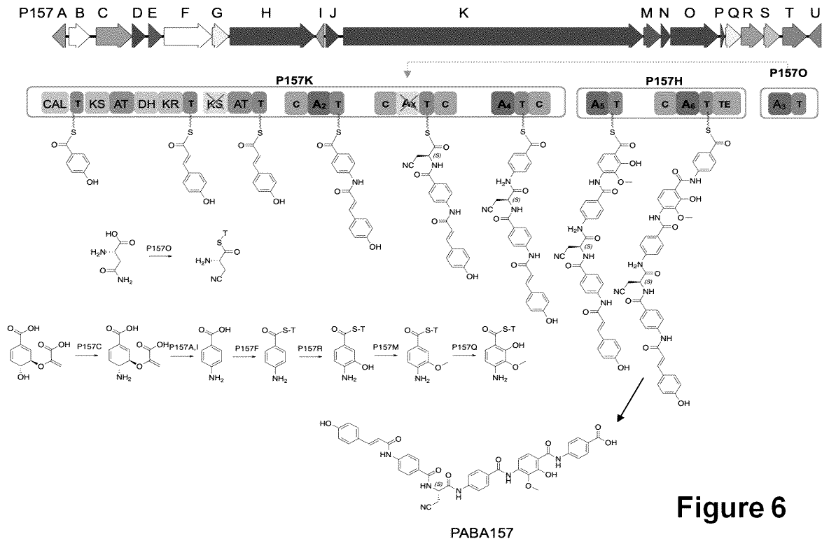

Key differences between cystobactamid and albicidin-like BGCs included the presence of a specific polyketide synthase module in albicidin BGCs and genes for the biosynthesis of 4-amino-2-hydroxy-3-isopropoxybenzoic acid (AHIBA) biosynthesis in cystobactamid BGCs (FIG. 3). Among the five hybrid PKS/NRPS BGCs identified, three distinct albicidin congeners were bioinformatically-derived. BGCs PABA34, PABA157 and PABA95 are representative of these three new structures. Three residues were positionally conserved across these congeners. This included a central β-L-cyanoalanine (CN-Ala), a 4-amino-2-hydroxy-3-methoxybenzoic acid (AHMBA) at the E position, and a PABA at the F position. Positions B and D contained different arrangements of PABA or AHMBA. The N-terminal methyl-coumaric acid (MCA) seen in albicidin was introduced by the PKS module that contained a ketosynthase (KS), dehydratase (DH), ketoreductase (KR), and methyltransferase (MT) domain. The corresponding PKS module in the PABA34 and PABA95 BGCs contained the same collection of domains (FIG. 4 and FIG. 5); however, in BGC PABA157 this module was missing the MT domain, indicating the biosynthesis of the coumaric acid (CA) in place of MCA (FIG. 6). This led to the indication that the products of PABA34 and PABA95 contained MCA at position A, while PABA157 incorporated a CA as the N-terminal building block.

Interestingly, no residue remained constant across these six bioinformatically-derived natural congeners. Although a growing number of analogs have been produced in synthetic efforts to improve the potency and resistance profile of this class of antibiotics, none of them matched the structures of these naturally selected congeners encoded by these metagenomic BGCs (Behroz I., 2019, Chem Eur J; Kerwat D et al., 2016, ChemMedChem, 11:1899-1903; Planke T et al., 2020, Chem Eur J, 26:4289-4296). Based on the present bioinformatic predictions, the total synthesis of each bioinformatically-derived congener was undertaken. These structures almost exclusively arose from the coupling of a shared set of substituted and unsubstituted PABA monomers. A convergent synthesis was therefore envisioned that enables facile access to all six structures from a minimum number of monomer building blocks.

In keeping with previous synthetic routes (Elgaher W A M et al., 2020, Chem Eur J, 26, 7219-7225; Huttel S et al., 2017, Angew Chem Int Ed Engl, 56:12760-12764; Moeller M et al., 2019, Org Lett, 21:8369-8372; Planke T et al., 2019, Org Lett, 21:1359-1363), each congener was disconnected retrosynthetically into three fragments: the N-terminal dipeptide, the central (3-methoxyasparagine or β-cyanoalanine unit, and the C-terminal tripeptide. FIG. 3C shows a representative retrosynthetic analysis of both a bioinfornatically-derived albicidin and cystobactamid congener. Utilization of this general strategy allowed to incorporate different bioinformatically-derived PABA building blocks at positions B, D, E and F as well as incorporate diverse building blocks at the N-terminal position. Integration of the central 3-L-cyanoalanine or β-methoxyasparagine units occurred strategically in each synthesis to minimize opportunities for racemization. Broad use of the allyl group was an integral part of the protecting group strategy of the phenol and ester functionalities in relevant PABA monomers. The final deprotected products, which were referred to as synthetic bioinformatic natural products (syn-BNPs) were HPLC purified and their structures were confirmed by HRMS as well as ¹H and ¹³C NMR spectroscopy.

All syn-BNPs, as well as the parent antibiotic albicidin were assayed for antimicrobial activity against the ESKAPE pathogens (Enterococcus faecium, Staphylococcus aureus, Klebsiella pneumoniae, Acinetobacter baumannii, Pseudomonas aeruginosa, and Enterobacter species), which are a collection of bacteria most commonly associated with antibacterial resistant nosocomial infections. Compared to albicidin, syn-BNPs showed improved potency against a number of pathogens (Table 9, Table 10) and differences in spectrum of activity, which is consistent for congeners that have evolved under different natural selective pressures.

TABLE 9
Syn-BNP MICs against ESKAPE pathogens and E. coli.

| | Gram- | | | | | | |
|---|---|---|---|---|---|---|---|
| ESKAPE | Positive | | Gram-negative | | | | |
| & | E. faecium | S. aureus | K. pneumoniae | | | E. cloacae | E. coli |
| E. coli | com15 | SH1000 | 10031 | A. baumannii | P. aeruginosa | 13047 | 25922 |
| PABA Compounds | | MIC (μg/mL) | | | | | |
| Albicidin | 2 | 0.5 | >8 | 0.125 | 1 | 8 | 0.015 |
| PABA48 | 0.125 | 0.25 | 0.125 | 0.125 | >8 | >8 | 0.125 |
| PABA70 | 1 | 1 | 1 | 4 | >8 | >8 | 2 |
| PABA57 | >8 | >8 | 1 | >8 | >8 | >8 | 2 |
| PABA34 | >8 | >8 | >8 | 2 | 2 | 2 | 0.0075 |
| PABA157 | >8 | 0.5 | >8 | 0.125 | 1 | 8 | 0.03 |
| PABA95 | 8 | 4 | >8 | 0.5 | 8 | 8 | 0.25 |
| Ciprofloxacin | 0.5 | 0.5 | 0.0019 | 0.125 | 0.125 | 0.06 | 0.0075 |

TABLE 10
Strain information.

| Group | Name of Bacteria | Strain |
|---|---|---|
| Firmicutes | Escherichia coli | ATCC25922 |
| | Escherichia coli_gyrA(D87G) | ATCC25922 |
| | Escherichia coli_gyrA(S83L) | ATCC25922 |
| | Escherichia coli | BL21(DE3) |
| ESKAPE pathogens | Enterococcus faecium | Com15 |
| | Staphylococcus aureus | SH1000 |
| | Klebsiella pneumonia | ATCC10031 |
| | Acinetobacter baumannii | ATCC17978 |
| | Pseudomonas aeruginosa | PAO1 |
| | Enterobacter cloacae | ATCC13047 |
| | Staphylococcus aureus | USA300 |
| | Staphylococcus aureus | ATCC BAA-42 |
| | Staphylococcus aureus | ATCC BAA-1721 |
| | Staphylococcus aureus | NRS146 |
| | Staphylococcus epidermidis | RP62A |
| | Enterococcus faecalis | 781 |
| | Enterococcus faecium | EF16 |
| | Enterococcus casseliflavus | 788 |

PABA34 showed the broadest potent Gram-negative activity with an MIC of <2 μg/mL against A. baumannii, P. aeruginosa, E. cloacea and E. coli (Table 9). PABA48 was the most potent broad-spectrum antibiotic we identified. It had the same MIC as albicidin against A. baumannii, but was 2, 16, and >64 fold more potent than albicidin against S. aureus, E. faecium and K. pneumoniae, respectively. Interestingly, unlike albicidin, all three cystobactamid analogs (PABA48, 70 and 57) showed potent activity against K. pneumoniae with MICs ranging from 0.125 to 1 μg/mL.

The most variable position among BGC-generated analogs was the A position, which involved four different building blocks (FIG. 3). PABA48, 70, and 57 are the first examples of a proteinogenic amino acid appearing at this position. This placed a positively charged amine at the N-terminus of the antibiotic. In contrast, all previously characterized congeners contained a neutral PABA or MCA at this position. The difference in potency between PABA48 and PABA70, which differed only by the proteinogenic amino acid at their N-termini, indicated this position was particularly important for potency. Traditional SAR studies have found that a small H-bond acceptor at the para position of the phenyl ring at this position is important for antibacterial activity (Testolin G. et al., 2020, Chemistry, 26:4289-4296; Testolin G. et al., 2020, Chem Sci, 11:1316-1334). As this was not found in the most potent broad spectrum analog PABA48, it indicated that there is an orthogonal strategy for increasing potency in this family of antibiotics.

### DNA Gyrase Resistance

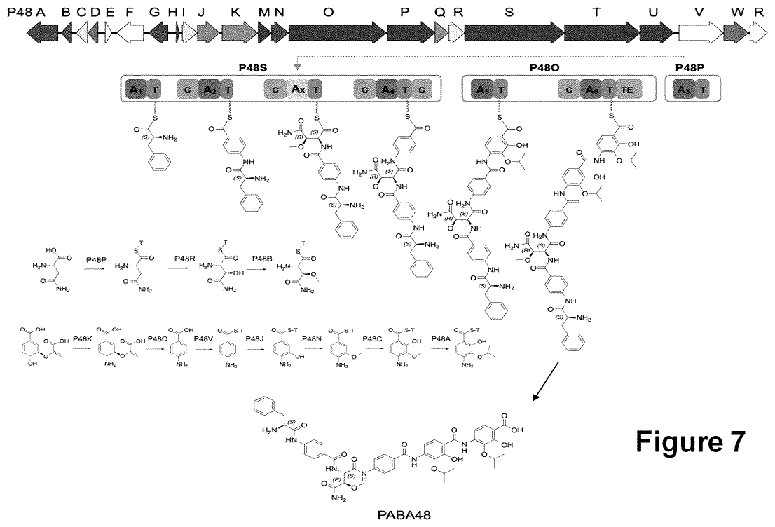

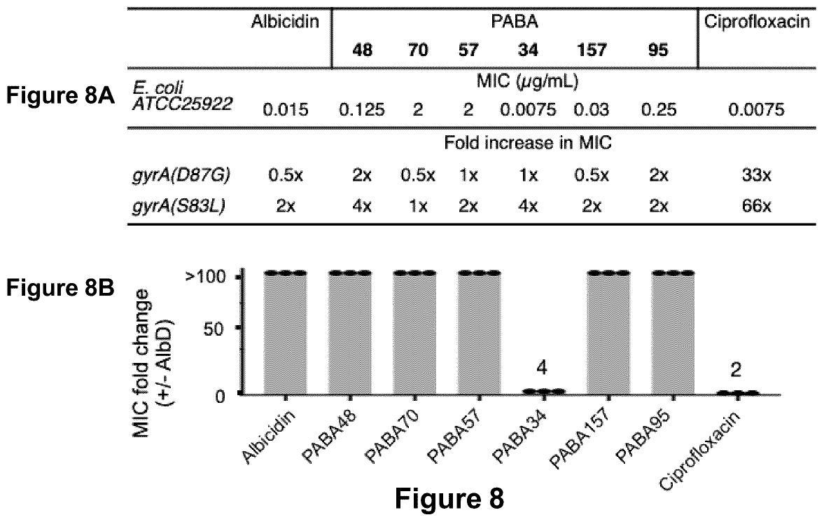

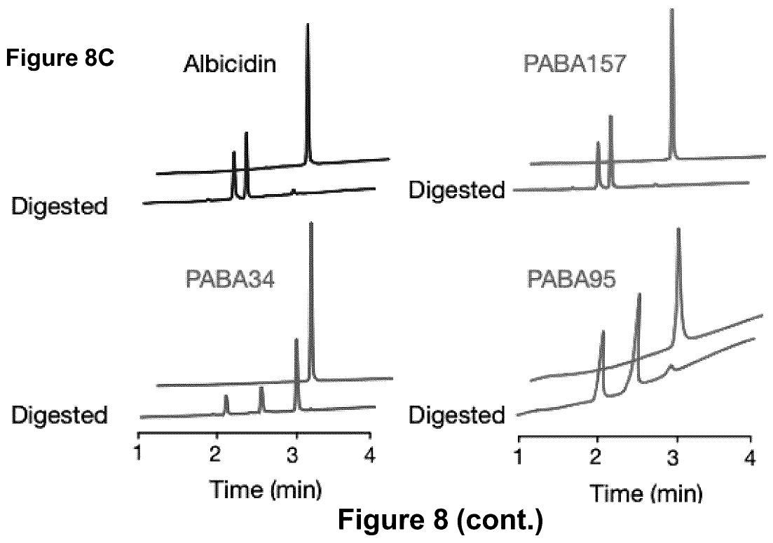

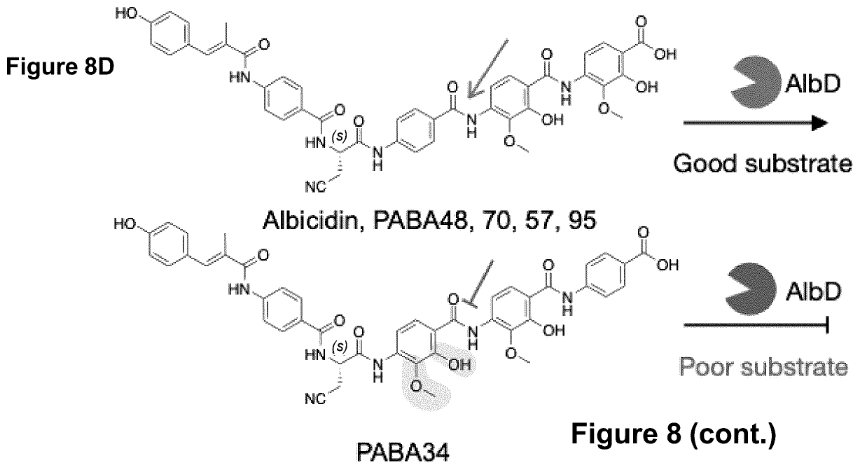

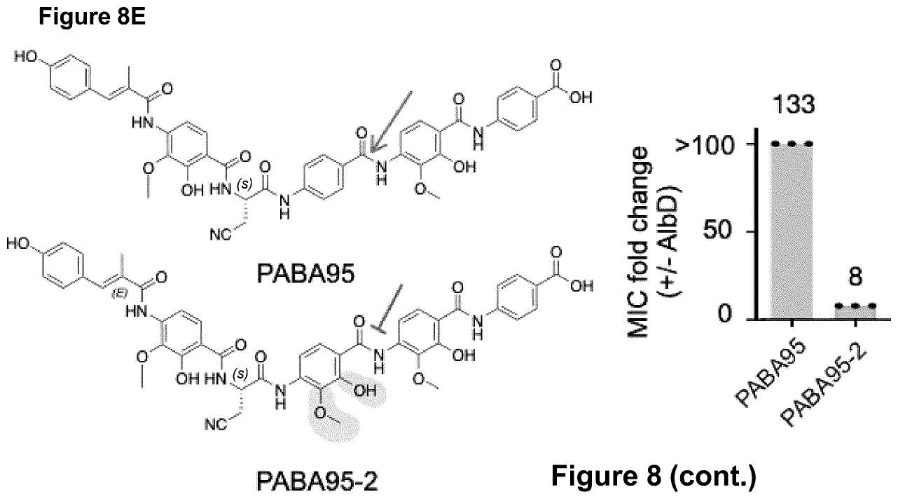

Consistent with cystobactamid and albicidin, all syn-BNPs inhibited DNA gyrase in vitro (FIG. 7). As mutations in DNA gyrase commonly confer resistance to DNA gyrase inhibitors, the present studies tested each syn-BNP against E. coli containing two different GyrA mutations (S83L, D87G) that are commonly seen in fluoroquinolone (i.e., ciprofloxacin) resistant clinical isolates (Bansal S et al., 2011, Int J Antimicrob Agents, 37:253-255). Neither mutation conferred cross resistance to any syn-BNP (FIG. 8A). Thus, the herein described analogs retained activity against existing problematic DNA gyrase variants. Further studies are focused on an investigation of other mutations via a clinical introduction of this class of antibiotics.

### AlbD Encoded Resistance

One key naturally occurring resistance mechanism for this class of antibiotics is the cleavage of the amide bond between the D and E monomers by AlbD-like endopeptidases (Vieweg L et al., 2015, J Am Chem Soc, 137:7608-7611; Walker M J et al., 1988, Mol Microbiol, 2:443-454). As circumventing common antibiotic resistance mechanisms was likely to play a key role in the natural selection of antibiotic congeners, the present studies investigated whether some of the congeners evolved to evade AlbD-mediated cleavage. To determine the susceptibility of the herein described BGC-inspired analogs to AlbD encoded resistance, the MIC of each syn-BNP against E. coli engineered to express AlbD was compared to that of E. coli containing an empty expression vector. All syn-BNPs, with the exception of PABA34 showed at least a 100-fold increase in MIC against the AlbD expressing strain (FIG. 8B, Table 11), suggesting that PABA34 was resistant to proteolytic cleavage by AlbD.

TABLE 11
MIC values of syn-BNPs against engineered E. coli.

| Bacteria | Albicidin | PABA48 | PABA70 | PABA57 | PABA34 | PABA157 | PABA95 | PABA95-2 | Ciprofloxacin |
|---|---|---|---|---|---|---|---|---|---|
| E. coli- | 0.0019 | 0.06 | 0.5 | 0.5 | 0.0075 | 0.0075 | 0.015 | 0.06 | 0.0009 |
| BL21 | | | | | | | | | |
| (pET28c) | | | | | | | | | |
| E. coli- | 0.25 | >8.0 | >64 | >64 | 0.03 | 1.0 | 2.0 | 0.5 | 0.0019 |
| BL21 | | | | | | | | | |
| (pET28c- | | | | | | | | | |
| AlbD) | | | | | | | | | |
| Fold | 131 | >133 | >128 | >128 | 4 | 133 | 133 | 8 | 2 |
| increase | | | | | | | | | |

As anticipated, incubation of PABA34 with purified AlbD revealed it was less susceptible to degradation than albicidin or any other syn-BNP (FIG. 8C). PABA34 was unique in that it contained the modified PABA, AHTMBA, at the D position directly adjacent to the endopeptidase cleavage site, indicating that this modification prevented hydrolysis by AlbD, thereby circumventing AlbD resistance (FIG. 8D). To test this, a version of PABA95 (PABA95-2), where the PABA at the D position was replaced with AHMBA, was synthesized. As seen with PABA34, this structure was not susceptible to AlbD encoded resistance (FIG. 8E).

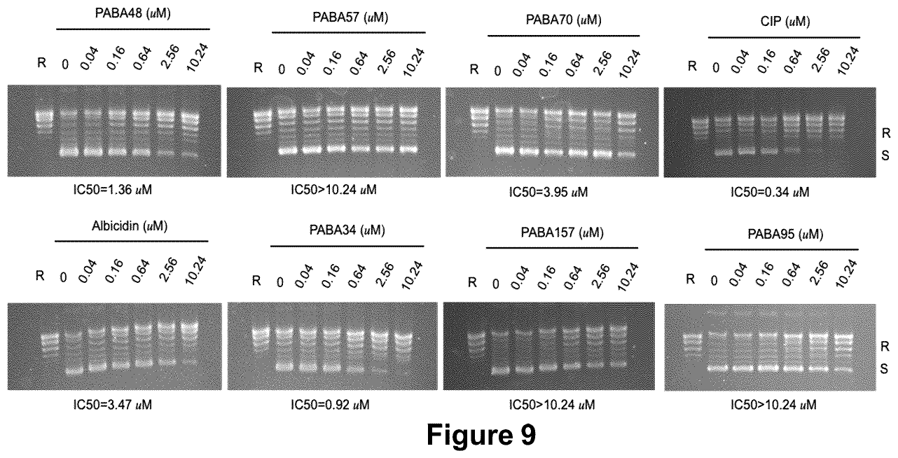

Traditional synthetic efforts to address AlbD resistance have focused primarily on replacing the amide bond at the AlbD cleavage site with bioisosteres (Behroz I et al., 2019, Chem Eur J; Planke T et al., 2020, Chem Eur J, 26:4289-4296; Testolin G et al., 2020, Chem Sci, 11:1316-1334). Although this provided resistance to AlbD cleavage, these structures often showed reduced antibiosis against a number of Gram-negative pathogens (Behroz I et al., 2019, Chem Eur J; Testolin G et al., 2020, Chem Sci, 11:1316-1334). In contrast, PABA34 was not only resistant to AlbD cleavage but also retained a high affinity for DNA gyrase (FIG. 9) and showed potent activity against most Gram-negative ESKAPE pathogens. Future synthetic efforts to further optimize resistance to AlbD cleavage while maximizing activity against Gram-negative pathogens benefit from mimicking the natural incorporation of a highly substituted PABA monomer (like AHMBA) at the D position.

Bioinformatic analysis of PABA encoding BGCs cloned from soil metagenomes guided the present synthesis of albicidin and cystobactamid analogs that demonstrated increased potency and different spectra of activity as well as the ability to circumvent resistance conferred by AlbD endopeptidase degradation. The present results highlighted the potential utility of metagenome derived BGCs to guide the synthesis of improved natural product variants, especially variants that overcome clinically relevant resistance while maintaining potent antibiosis. In contrast to traditional medicinal chemistry approaches for improving natural product lead structures, the combination of metagenomic discovery methods with bioinformatic natural product prediction methods to generate synthetic targets offers the opportunity to identify improved antibiotics that have evolved under the pressure of natural selection to be biologically active. The general approach presented here of using congener BGCs to guide medicinal chemistry studies represented a broadly applicable method for intelligently refining existing natural products to improve potency, spectrum of activity, or susceptibility to common resistance mechanism, as differences in these features are likely to arise from the same environmental pressures that drive the selection of naturally occurring congeners.

In conclusion, natural products are a major source of new antibiotics. Efforts to improve a natural antibiotic's activity often rely on the synthesis of libraries of structurally diverse analogs. The present studies showed that biosynthetic instructions contained within metagenome-derived congener BGCs are used to guide the synthesis of improved antibiotic analogs. Albicidin and cystobactamid were the first members of a new class of broad spectrum PABA-based antibiotics. The herein described search for PABA specific adenylation domain sequences in soil metagenomes revealed that BGCs in this family were common in nature. Twelve BGCs that were bioinformatically-derived to encode six new congeners were recovered from soil metagenomic libraries. Total chemical synthesis of these six bioinformatically-derived structures led to our identification of potent antibiotics with changes in spectrum of activity and most interestingly, the ability to circumvent resistance conferred by endopeptidase cleavage enzymes.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A compound represented by Formula (I) 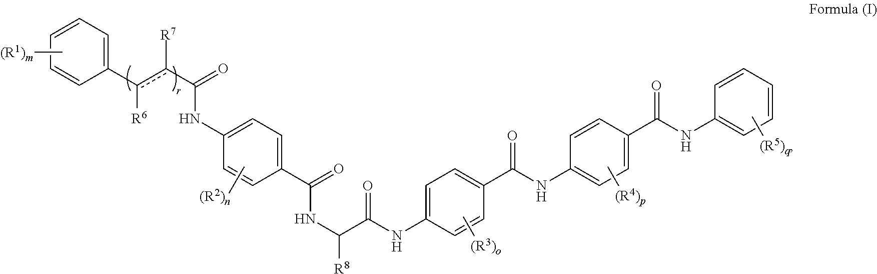
or a racemate, an enantiomer, a diastereomer, a pharmaceutically acceptable salt, or a derivative thereof,
wherein each R¹, R², R³, R⁴, R⁵, R⁶, R⁷, and R⁸ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof;
each m and q independently represents an integer of 0 to 5;
each n, o, and p independently represents an integer of 0 to 4; and
r represents an integer of 0 or 1.

2. The compound of claim 1, wherein each R¹, R², R³, R⁴, R⁵, R⁶, R⁷, and R⁹ is independently selected from the group consisting of hydrogen, alkyl, amino, amido, hydroxyl, hydroxyalkyl, carboxyl, —CN, and any combination thereof.

3. The compound of claim 2, wherein each R¹, R², R³, R⁴, R⁵, R⁶, R⁷, and R⁸ is independently selected from the group consisting of hydrogen, methyl, amino, hydroxyl, carboxyl,

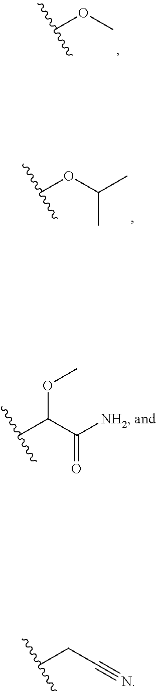

4. The compound of claim 1, wherein the compound represented by Formula (I) is a compound selected from the group consisting of: 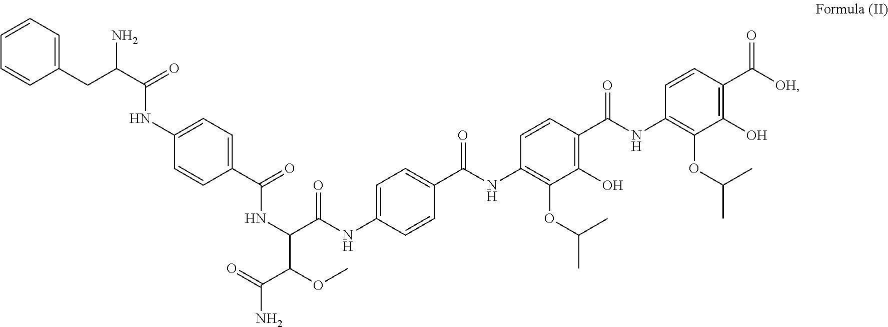 [SMILES: COC(C(N)=O)C(NC(=O)c1ccc(NC(=O)C(N)Cc2ccccc2)cc1)C(=O)Nc1ccc(C(=O)Nc2ccc(C(=O)Nc3ccc(C(=O)O)c(O)c3OC(C)C)c(O)c2OC(C)C)cc1]
or a racemate, an enantiomer, a diastereomer, a pharmaceutically acceptable salt, or a derivative thereof, 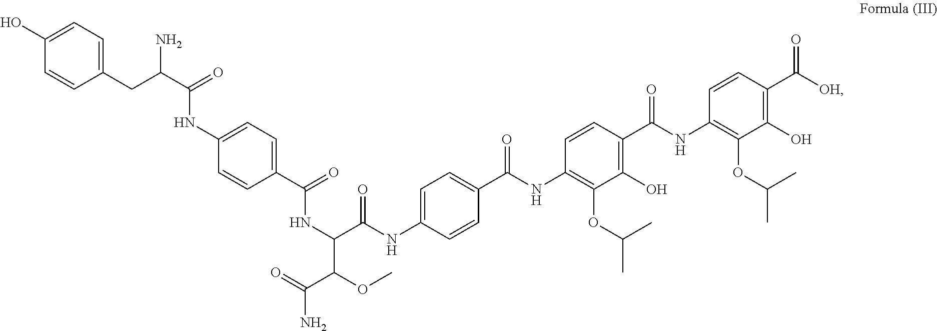 [SMILES: COC(C(N)=O)C(NC(=O)c1ccc(NC(=O)C(N)Cc2ccc(O)cc2)cc1)C(=O)Nc1ccc(C(=O)Nc2ccc(C(=O)Nc3ccc(C(=O)O)c(O)c3OC(C)C)c(O)c2OC(C)C)cc1]
or a racemate, an enantiomer, a diastereomer, a pharmaceutically acceptable salt, or a derivative thereof, 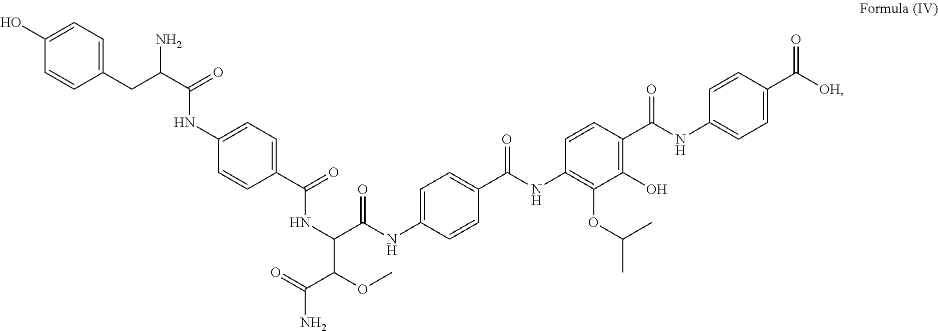 [SMILES: COC(C(N)=O)C(NC(=O)c1ccc(NC(=O)C(N)Cc2ccc(O)cc2)cc1)C(=O)Nc1ccc(C(=O)Nc2ccc(C(=O)Nc3ccc(C(=O)O)cc3)c(O)c2OC(C)C)cc1]
or a racemate, an enantiomer, a diastereomer, a pharmaceutically acceptable salt, or a derivative thereof, 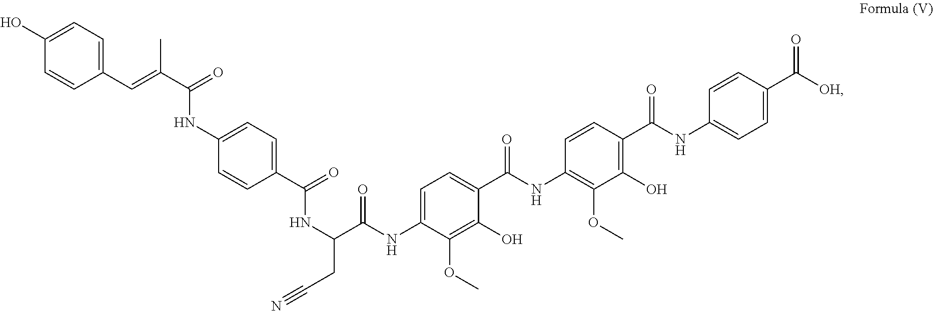 [SMILES: COc1c(NC(=O)c2ccc(NC(=O)C(CC#N)NC(=O)c3ccc(NC(=O)/C(C)=C/c4ccc(O)cc4)cc3)c(OC)c2O)ccc(C(=O)Nc2ccc(C(=O)O)cc2)c1O]
or a racemate, an enantiomer, a diastereomer, a pharmaceutically acceptable salt, or a derivative thereof, 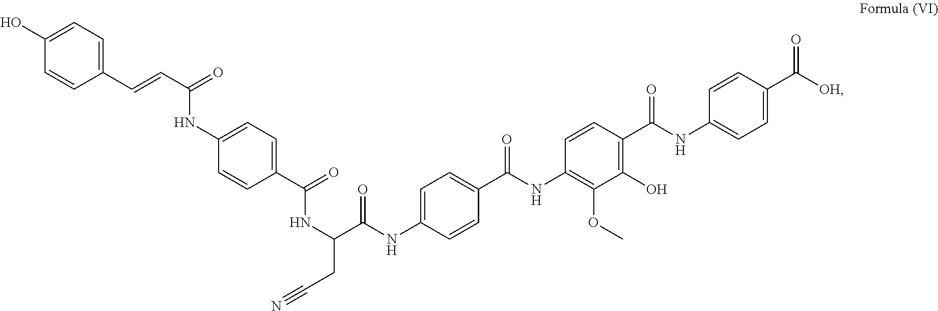 [SMILES: COc1c(NC(=O)c2ccc(NC(=O)C(CC#N)NC(=O)c3ccc(NC(=O)/C=C/c4ccc(O)cc4)cc3)cc2)ccc(C(=O)Nc2ccc(C(=O)O)cc2)c1O]
or a racemate, an enantiomer, a diastereomer, a pharmaceutically acceptable salt, or a derivative thereof, 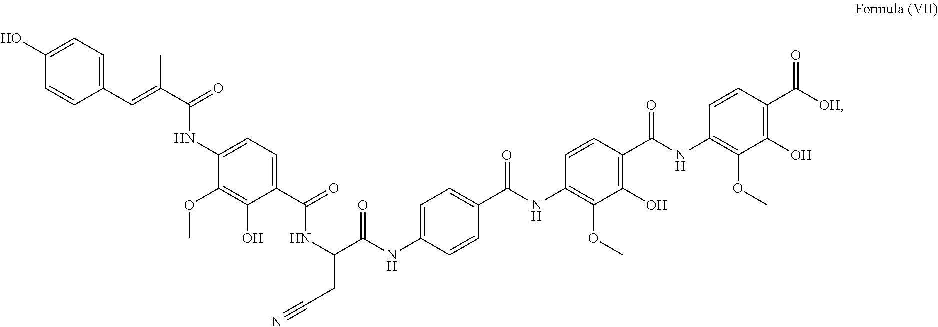 [SMILES: COc1c(NC(=O)c2ccc(NC(=O)c3ccc(NC(=O)C(CC#N)NC(=O)c4ccc(NC(=O)/C(C)=C/c5ccc(O)cc5)c(OC)c4O)cc3)c(OC)c2O)ccc(C(=O)O)c1O]
or a racemate, an enantiomer, a diastereomer, a pharmaceutically acceptable salt, or a derivative thereof, 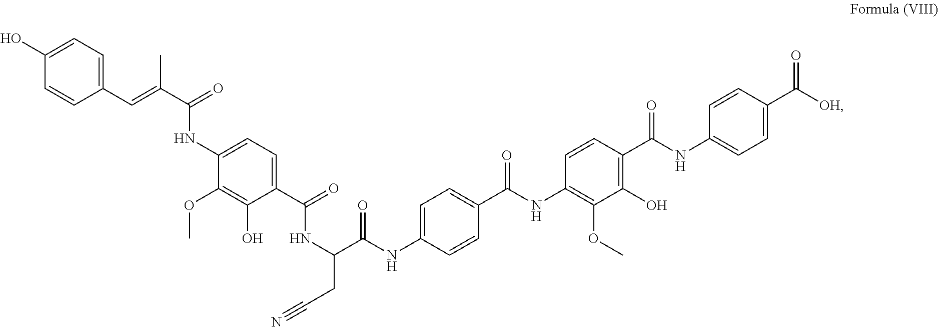 [SMILES: COc1c(NC(=O)/C(C)=C/c2ccc(O)cc2)ccc(C(=O)NC(CC#N)C(=O)Nc2ccc(C(=O)Nc3ccc(C(=O)Nc4ccc(C(=O)O)cc4)c(O)c3OC)cc2)c1O]
or a racemate, an enantiomer, a diastereomer, a pharmaceutically acceptable salt, or a derivative thereof, and 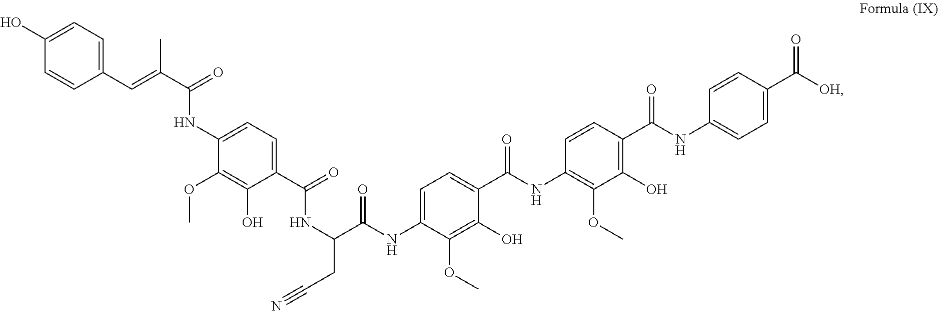 [SMILES: COc1c(NC(=O)/C(C)=C/c2ccc(O)cc2)ccc(C(=O)NC(CC#N)C(=O)Nc2ccc(C(=O)Nc3ccc(C(=O)Nc4ccc(C(=O)O)cc4)c(O)c3OC)c(O)c2OC)c1O]
or a racemate, an enantiomer, a diastereomer, a pharmaceutically acceptable salt, or a derivative thereof.

5. The compound of claim 1, wherein the compound represented by Formula (I) is a compound selected from the group consisting of: 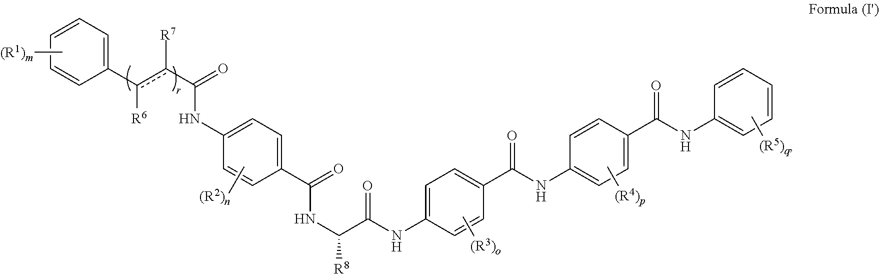
or a racemate, an enantiomer, a diastereomer, a pharmaceutically acceptable salt, or a derivative thereof, and 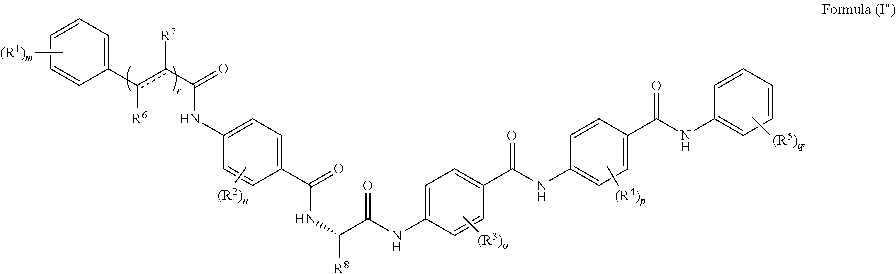
or a racemate, an enantiomer, a diastereomer, a pharmaceutically acceptable salt, or a derivative thereof,
wherein each R¹, R², R³, R⁴, R⁵, R⁶, R⁷, and R⁸ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof;
each m and q independently represents an integer of 0 to 5;
each n, o, and p independently represents an integer of 0 to 4; and
r represents an integer of 0 or 1.

6. The compound of claim 5, wherein each R¹, R², R³, R⁴, R⁵, R⁶, R⁷, and R⁸ is independently selected from the group consisting of hydrogen, alkyl, amino, amido, hydroxyl, hydroxyalkyl, carboxyl, —CN, and any combination thereof.

7. The compound of claim 6, wherein each R¹, R², R³, R⁴, R⁵, R⁶, R⁷, and R⁸ is independently selected from the group consisting of hydrogen, methyl, amino, hydroxyl, carboxyl,

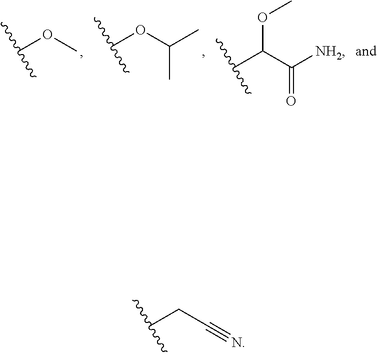

8. The compound of claim 1, wherein the compound represented by Formula (I) is a compound selected from the group consisting of: 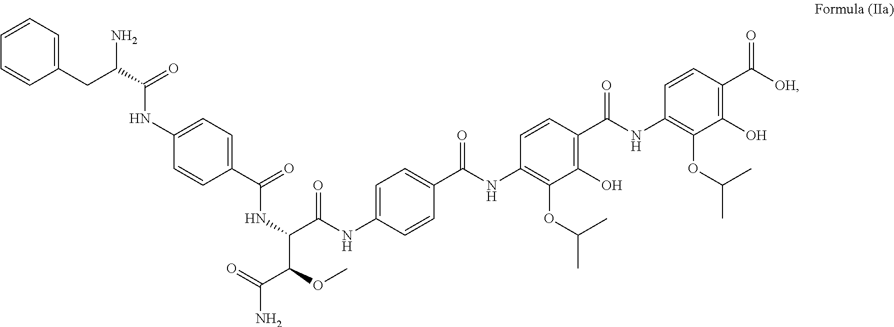
or a racemate, an enantiomer, a diastereomer, a pharmaceutically acceptable salt, or a derivative thereof, 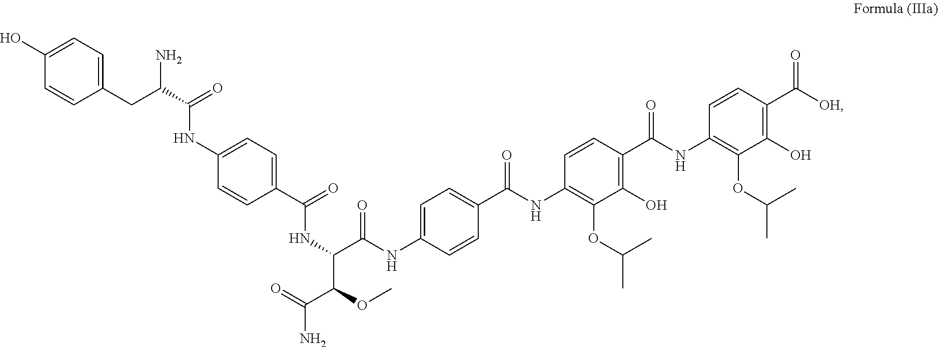
or a racemate, an enantiomer, a diastereomer, a pharmaceutically acceptable salt, or a derivative thereof, 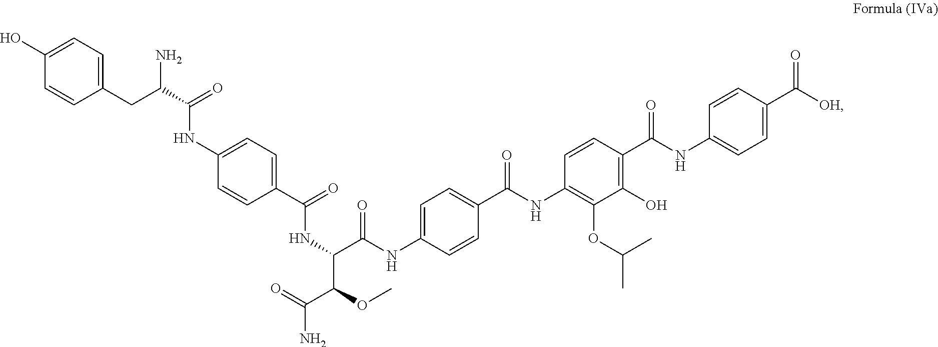
or a racemate, an enantiomer, a diastereomer, a pharmaceutically acceptable salt, or a derivative thereof, 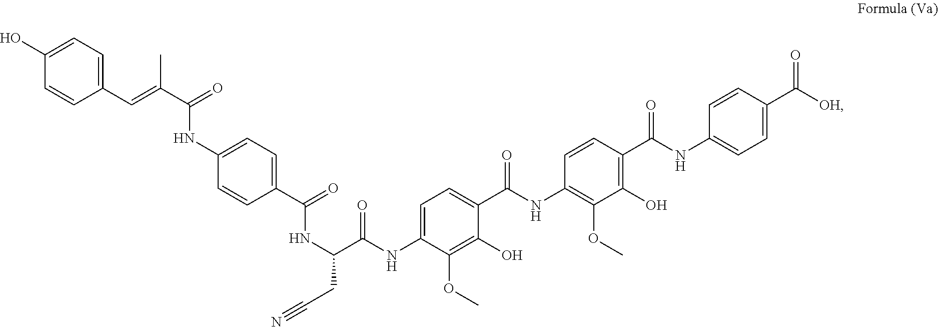 [SMILES: COc1c(NC(=O)c2ccc(NC(=O)[C@H](CC#N)NC(=O)c3ccc(NC(=O)/C(C)=C/c4ccc(O)cc4)cc3)c(OC)c2O)ccc(C(=O)Nc2ccc(C(=O)O)cc2)c1O]
or a racemate, an enantiomer, a diastereomer, a pharmaceutically acceptable salt, or a derivative thereof, 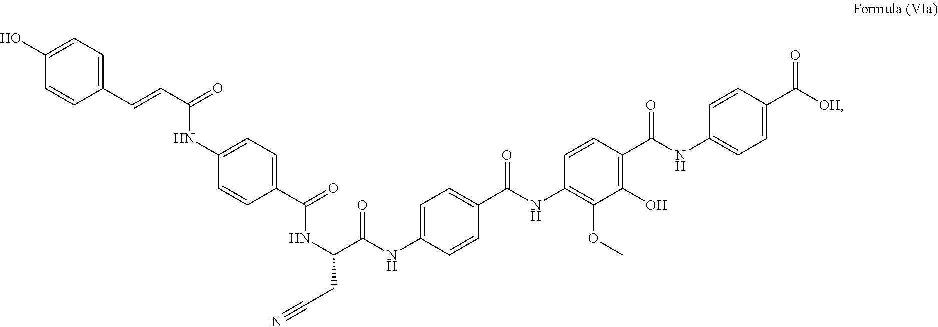 [SMILES: COc1c(NC(=O)c2ccc(NC(=O)[C@H](CC#N)NC(=O)c3ccc(NC(=O)/C=C/c4ccc(O)cc4)cc3)cc2)ccc(C(=O)Nc2ccc(C(=O)O)cc2)c1O]
or a racemate, an enantiomer, a diastereomer, a pharmaceutically acceptable salt, or a derivative thereof, 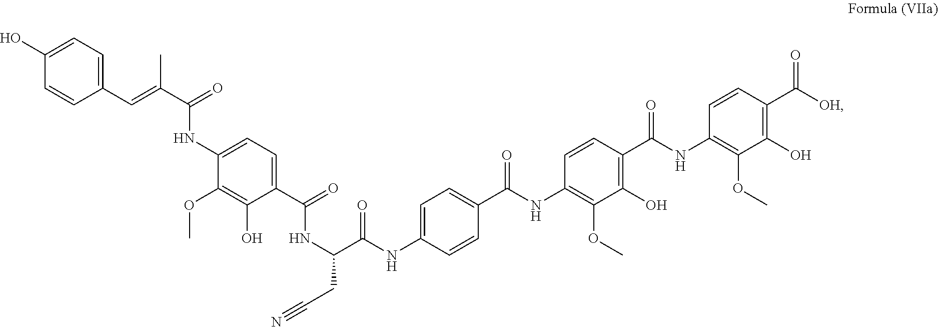 [SMILES: COc1c(NC(=O)c2ccc(NC(=O)c3ccc(NC(=O)[C@H](CC#N)NC(=O)c4ccc(NC(=O)/C(C)=C/c5ccc(O)cc5)c(OC)c4O)cc3)c(OC)c2O)ccc(C(=O)O)c1O]
or a racemate, an enantiomer, a diastereomer, a pharmaceutically acceptable salt, or a derivative thereof, 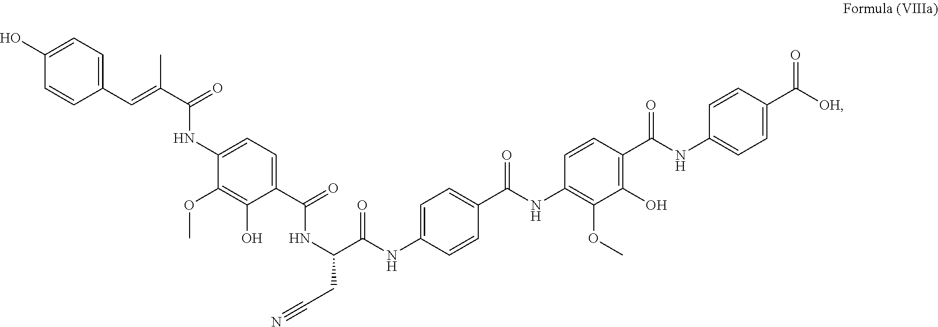 [SMILES: COc1c(NC(=O)/C(C)=C/c2ccc(O)cc2)ccc(C(=O)N[C@@H](CC#N)C(=O)Nc2ccc(C(=O)Nc3ccc(C(=O)Nc4ccc(C(=O)O)cc4)c(O)c3OC)cc2)c1O]
or a racemate, an enantiomer, a diastereomer, a pharmaceutically acceptable salt, or a derivative thereof, and 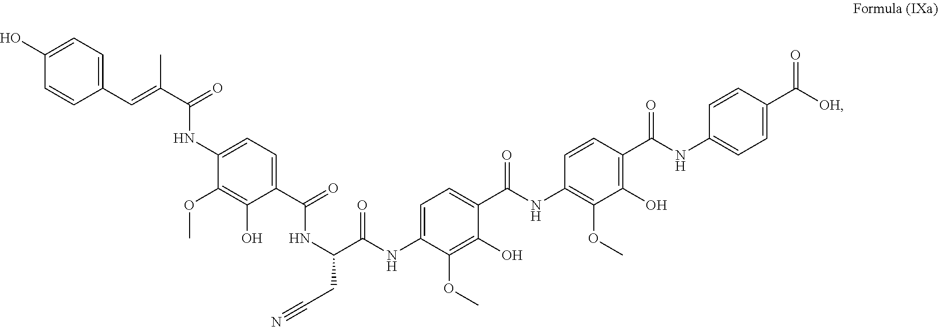 [SMILES: COc1c(NC(=O)/C(C)=C/c2ccc(O)cc2)ccc(C(=O)N[C@@H](CC#N)C(=O)Nc2ccc(C(=O)Nc3ccc(C(=O)Nc4ccc(C(=O)O)cc4)c(O)c3OC)c(O)c2OC)c1O]
or a racemate, an enantiomer, a diastereomer, a pharmaceutically acceptable salt, or a derivative thereof.

9. A composition comprising one or more compounds of claim 1.

10. The composition of claim 9, wherein the composition is a pharmaceutical composition.

11. An isolated nucleic acid encoding a compound represented by Formula (I) 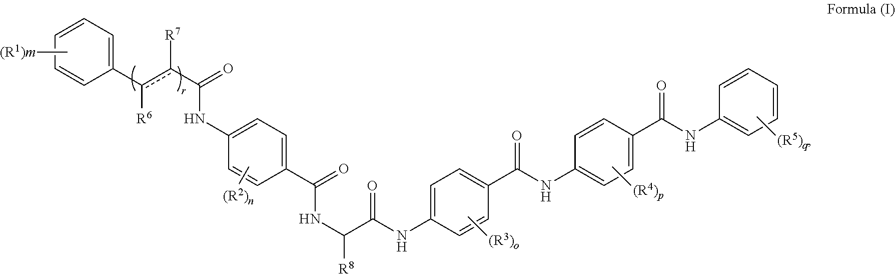
or a racemate, an enantiomer, a diastereomer, a pharmaceutically acceptable salt, or a derivative thereof,
wherein each R¹, R², R³, R⁴, R⁵, R⁶, R⁷, and R⁸ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof;
each m and q independently represents an integer of 0 to 5;
each n, o, and p independently represents an integer of 0 to 4; and
r represents an integer of 0 or 1.

12. The isolated nucleic acid of claim 11, wherein each R¹, R², R³, R⁴, R⁵, R⁶, R⁷, and R⁸ is independently selected from the group consisting of hydrogen, alkyl, amino, amido, hydroxyl, hydroxyalkyl, carboxyl, —CN, and any combination thereof.

13. The isolated nucleic acid of claim 12, wherein each R¹, R², R³, R⁴, R⁵, R⁶, R⁷, and R⁸ is independently selected from the group consisting of hydrogen, methyl, amino, hydroxyl, carboxyl,

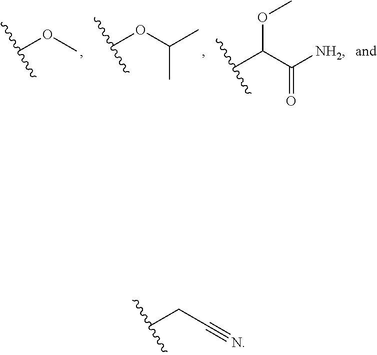

14. The isolated nucleic acid of claim 11, wherein the compound represented by Formula (I) is a compound selected from the group consisting of: 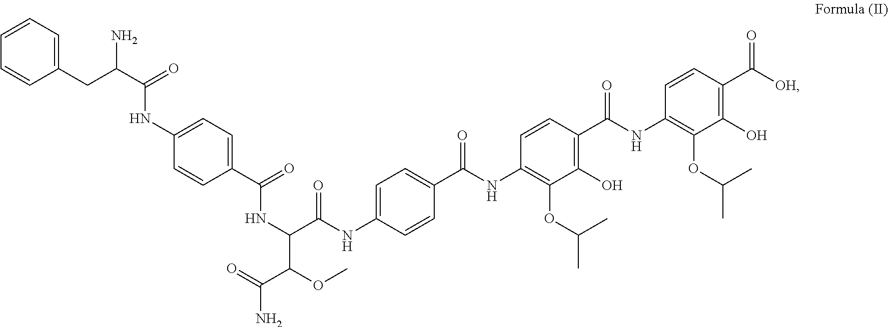 [SMILES: COC(C(N)=O)C(NC(=O)c1ccc(NC(=O)C(N)Cc2ccccc2)cc1)C(=O)Nc1ccc(C(=O)Nc2ccc(C(=O)Nc3ccc(C(=O)O)c(O)c3OC(C)C)c(O)c2OC(C)C)cc1]
or a racemate, an enantiomer, a diastereomer, a pharmaceutically acceptable salt, or a derivative thereof, 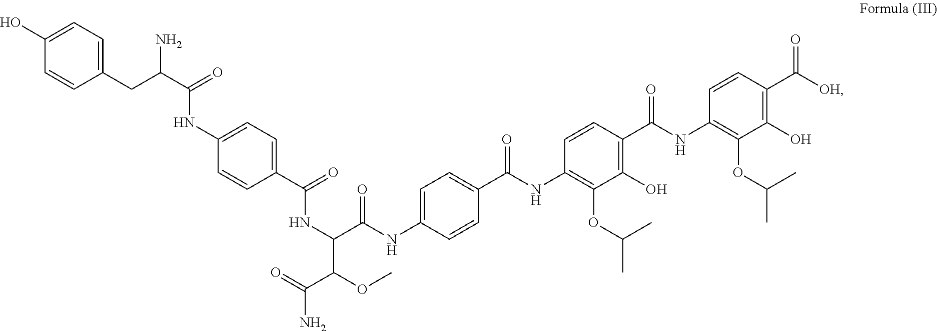 [SMILES: COC(C(N)=O)C(NC(=O)c1ccc(NC(=O)C(N)Cc2ccc(O)cc2)cc1)C(=O)Nc1ccc(C(=O)Nc2ccc(C(=O)Nc3ccc(C(=O)O)c(O)c3OC(C)C)c(O)c2OC(C)C)cc1]
or a racemate, an enantiomer, a diastereomer, a pharmaceutically acceptable salt, or a derivative thereof, 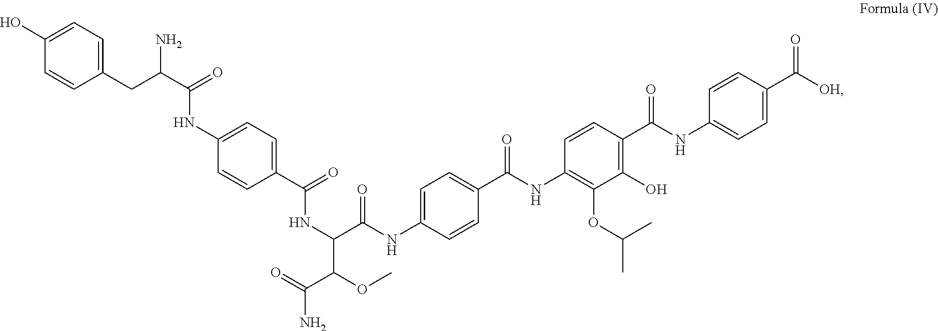 [SMILES: COC(C(N)=O)C(NC(=O)c1ccc(NC(=O)C(N)Cc2ccc(O)cc2)cc1)C(=O)Nc1ccc(C(=O)Nc2ccc(C(=O)Nc3ccc(C(=O)O)cc3)c(O)c2OC(C)C)cc1]
or a racemate, an enantiomer, a diastereomer, a pharmaceutically acceptable salt, or a derivative thereof, 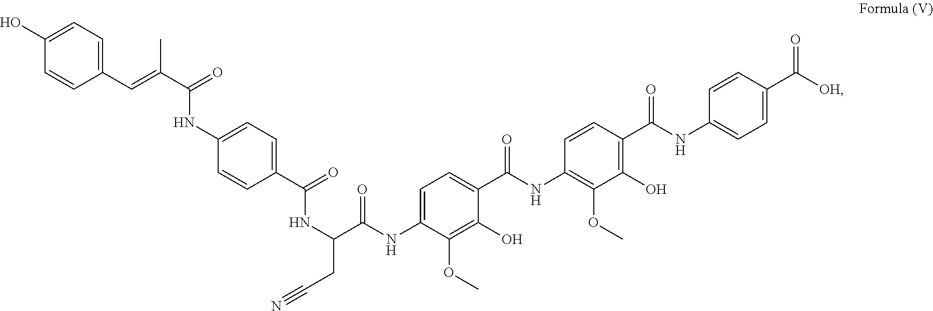 [SMILES: COc1c(NC(=O)c2ccc(NC(=O)C(CC#N)NC(=O)c3ccc(NC(=O)/C(C)=C/c4ccc(O)cc4)cc3)c(OC)c2O)ccc(C(=O)Nc2ccc(C(=O)O)cc2)c1O]
or a racemate, an enantiomer, a diastereomer, a pharmaceutically acceptable salt, or a derivative thereof, 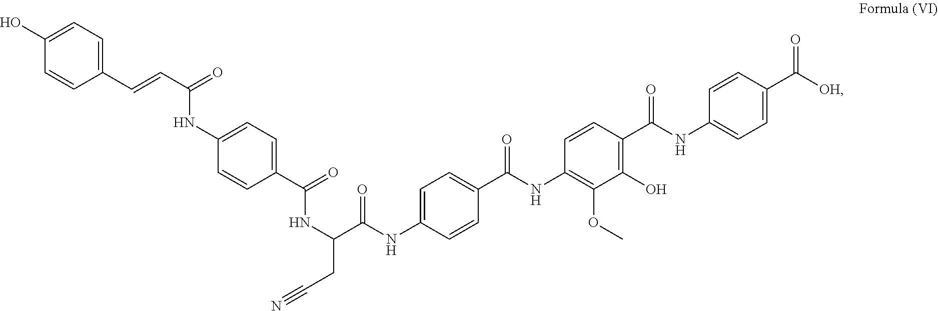 [SMILES: COc1c(NC(=O)c2ccc(NC(=O)C(CC#N)NC(=O)c3ccc(NC(=O)/C=C/c4ccc(O)cc4)cc3)cc2)ccc(C(=O)Nc2ccc(C(=O)O)cc2)c1O]
or a racemate, an enantiomer, a diastereomer, a pharmaceutically acceptable salt, or a derivative thereof, 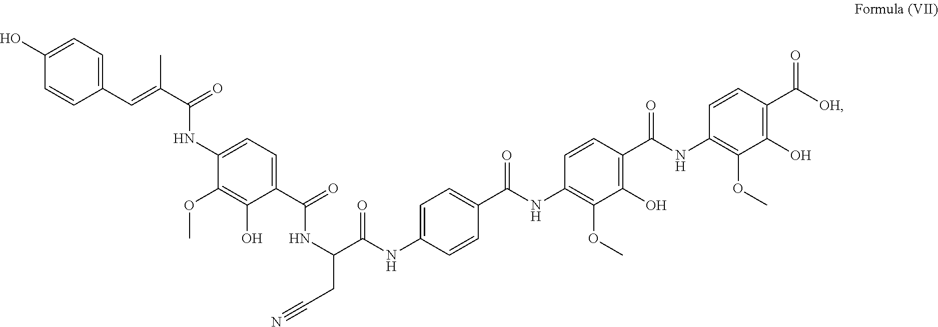 [SMILES: COc1c(NC(=O)c2ccc(NC(=O)c3ccc(NC(=O)C(CC#N)NC(=O)c4ccc(NC(=O)/C(C)=C/c5ccc(O)cc5)c(OC)c4O)cc3)c(OC)c2O)ccc(C(=O)O)c1O]
or a racemate, an enantiomer, a diastereomer, a pharmaceutically acceptable salt, or a derivative thereof, 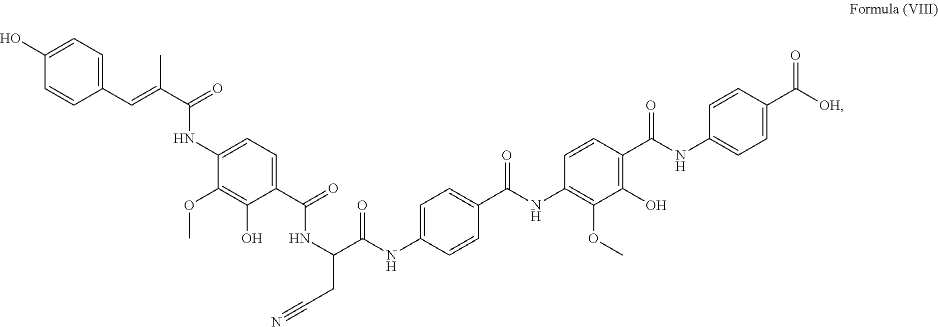 [SMILES: COc1c(NC(=O)/C(C)=C/c2ccc(O)cc2)ccc(C(=O)NC(CC#N)C(=O)Nc2ccc(C(=O)Nc3ccc(C(=O)Nc4ccc(C(=O)O)cc4)c(O)c3OC)cc2)c1O]
or a racemate, an enantiomer, a diastereomer, a pharmaceutically acceptable salt, or a derivative thereof, and 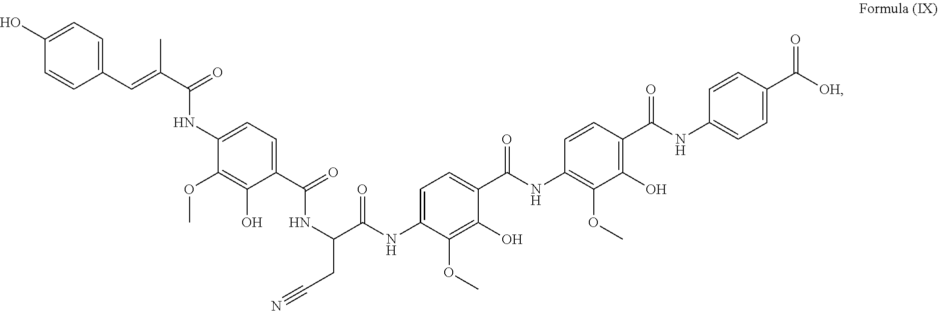 [SMILES: COc1c(NC(=O)/C(C)=C/c2ccc(O)cc2)ccc(C(=O)NC(CC#N)C(=O)Nc2ccc(C(=O)Nc3ccc(C(=O)Nc4ccc(C(=O)O)cc4)c(O)c3OC)c(O)c2OC)c1O]
or a racemate, an enantiomer, a diastereomer, a pharmaceutically acceptable salt, or a derivative thereof.

15. The isolated nucleic acid of claim 11, wherein the compound represented by Formula (I) is a compound selected from the group consisting of: 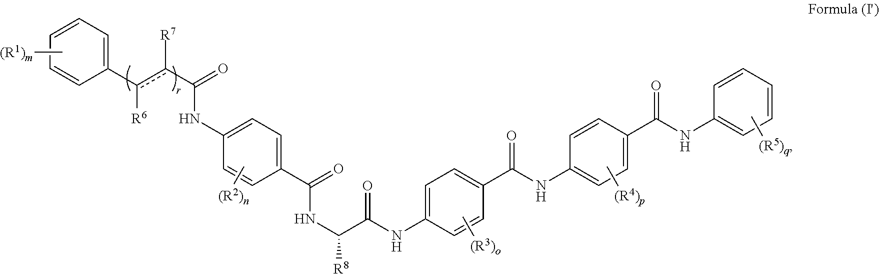
or a racemate, an enantiomer, a diastereomer, a pharmaceutically acceptable salt, or a derivative thereof, and 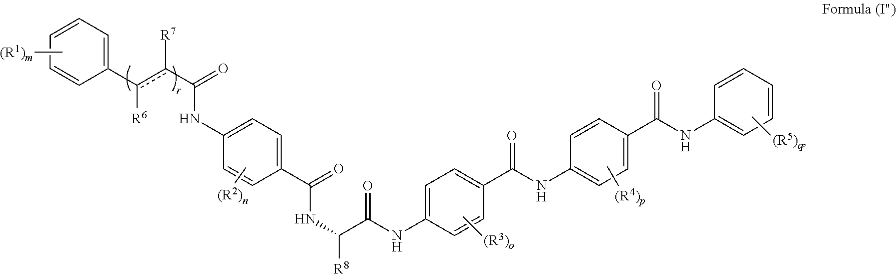
or a racemate, an enantiomer, a diastereomer, a pharmaceutically acceptable salt, or a derivative thereof,
wherein each R¹, R², R³, R⁴, R⁵, R⁶, R⁷, and R⁸ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof;
each m and q independently represents an integer of 0 to 5;
each n, o, and p independently represents an integer of 0 to 4; and
r represents an integer of 0 or 1.

16. The isolated nucleic acid of claim 15, wherein each R¹, R², R³, R⁴, R⁵, R⁶, R⁷, and R⁸ is independently selected from the group consisting of hydrogen, alkyl, amino, amido, hydroxyl, hydroxyalkyl, carboxyl, —CN, and any combination thereof.

17. The isolated nucleic acid of claim 16, wherein each R¹, R², R³, R⁴, R⁵, R⁶, R⁷, and R⁸ is independently selected from the group consisting of hydrogen, methyl, amino, hydroxyl, carboxyl,

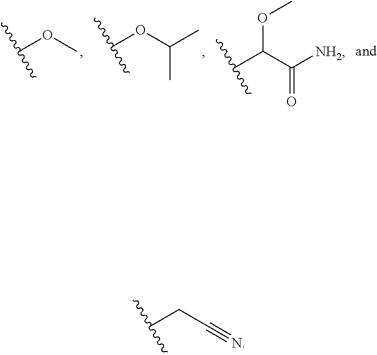

18. The isolated nucleic acid of claim 1, wherein the compound represented by Formula (I) is a compound selected from the group consisting of: 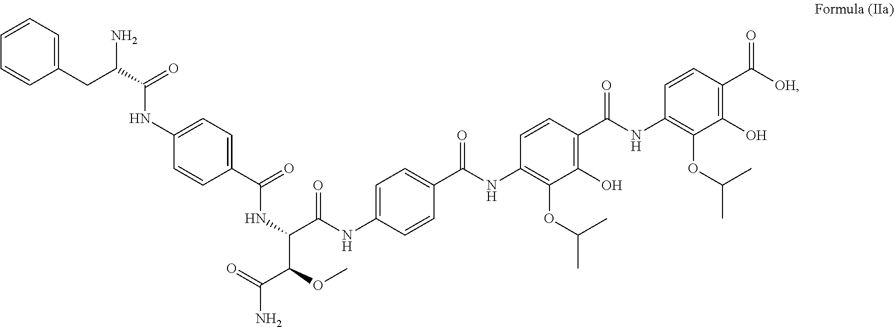
or a racemate, an enantiomer, a diastereomer, a pharmaceutically acceptable salt, or a derivative thereof, 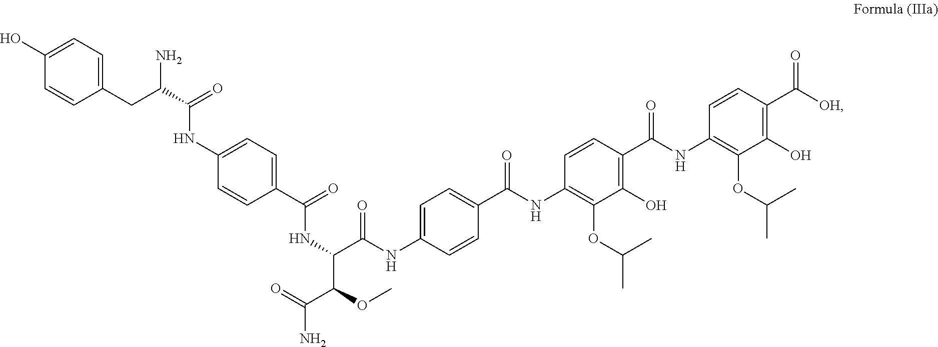
or a racemate, an enantiomer, a diastereomer, a pharmaceutically acceptable salt, or a derivative thereof, 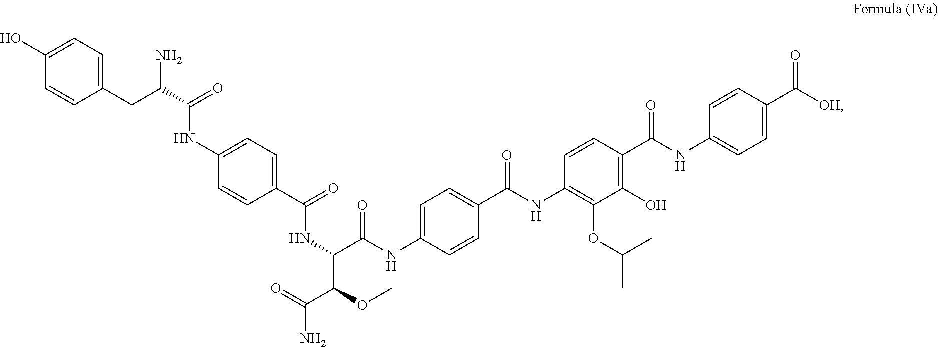
or a racemate, an enantiomer, a diastereomer, a pharmaceutically acceptable salt, or a derivative thereof, 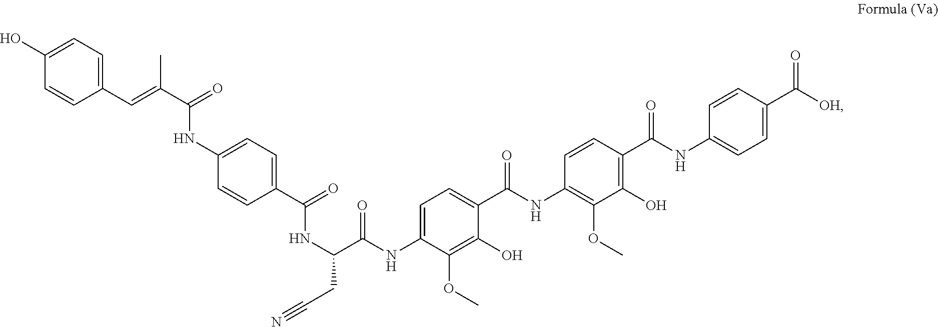 [SMILES: COc1c(NC(=O)c2ccc(NC(=O)[C@H](CC#N)NC(=O)c3ccc(NC(=O)/C(C)=C/c4ccc(O)cc4)cc3)c(OC)c2O)ccc(C(=O)Nc2ccc(C(=O)O)cc2)c1O]
or a racemate, an enantiomer, a diastereomer, a pharmaceutically acceptable salt, or a derivative thereof, 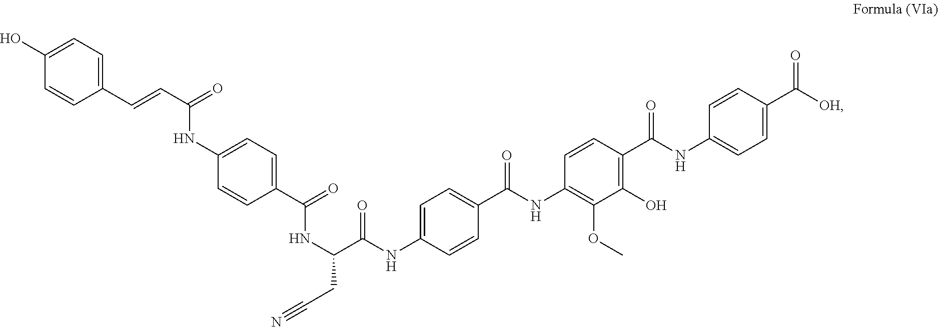 [SMILES: COc1c(NC(=O)c2ccc(NC(=O)[C@H](CC#N)NC(=O)c3ccc(NC(=O)/C=C/c4ccc(O)cc4)cc3)cc2)ccc(C(=O)Nc2ccc(C(=O)O)cc2)c1O]
or a racemate, an enantiomer, a diastereomer, a pharmaceutically acceptable salt, or a derivative thereof, 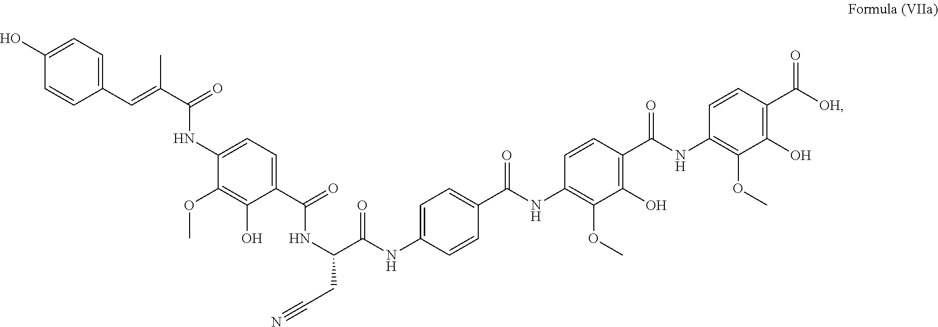 [SMILES: COc1c(NC(=O)c2ccc(NC(=O)c3ccc(NC(=O)[C@H](CC#N)NC(=O)c4ccc(NC(=O)/C(C)=C/c5ccc(O)cc5)c(OC)c4O)cc3)c(OC)c2O)ccc(C(=O)O)c1O]
or a racemate, an enantiomer, a diastereomer, a pharmaceutically acceptable salt, or a derivative thereof, 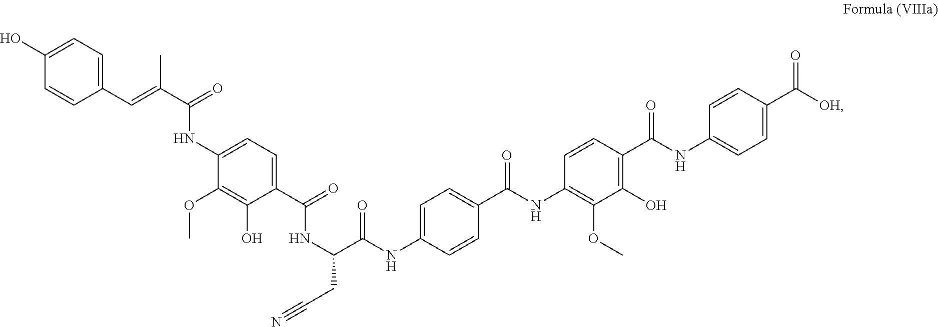 [SMILES: COc1c(NC(=O)/C(C)=C/c2ccc(O)cc2)ccc(C(=O)N[C@@H](CC#N)C(=O)Nc2ccc(C(=O)Nc3ccc(C(=O)Nc4ccc(C(=O)O)cc4)c(O)c3OC)cc2)c1O]
or a racemate, an enantiomer, a diastereomer, a pharmaceutically acceptable salt, or a derivative thereof, and 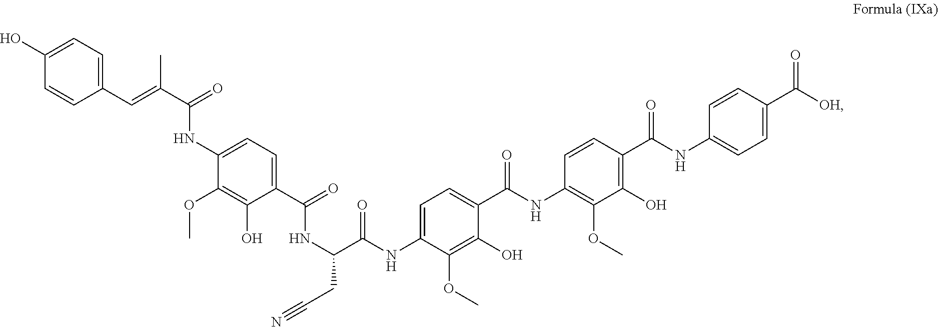 [SMILES: COc1c(NC(=O)/C(C)=C/c2ccc(O)cc2)ccc(C(=O)N[C@@H](CC#N)C(=O)Nc2ccc(C(=O)Nc3ccc(C(=O)Nc4ccc(C(=O)O)cc4)c(O)c3OC)c(O)c2OC)c1O]
or a racemate, an enantiomer, a diastereomer, a pharmaceutically acceptable salt, or a derivative thereof.

19. A genetically engineered cell, wherein the cell expresses one or more compounds represented by Formula (I) 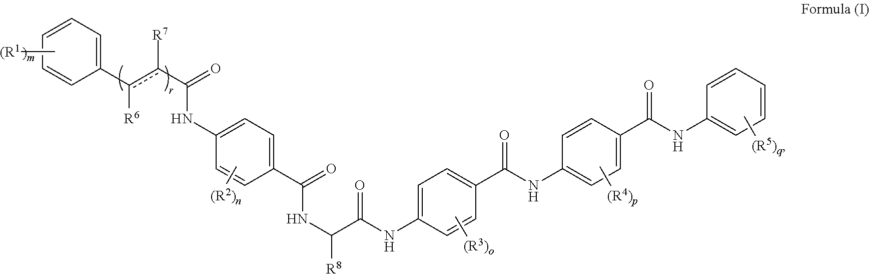
or a racemate, an enantiomer, a diastereomer, a pharmaceutically acceptable salt, or a derivative thereof,
wherein each R¹, R², R³, R⁴, R⁵, R⁶, R⁷, and R⁸ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof;
each m and q independently represents an integer of 0 to 5;
each n, o, and p independently represents an integer of 0 to 4; and
r represents an integer of 0 or 1.

20. The cell of claim 19, wherein the cell is transformed with the nucleic acid of claim 11.

21. A method of treating or preventing a bacterial infection in a subject in need thereof, the method comprising administering a composition comprising a compound of claim 1 to the subject.

22. The method of claim 21, wherein the subject is exposed to or infected with a bacteria.

23. The method of claim 22, wherein the bacteria is a gram positive bacteria.

24. The method of claim 22, wherein the bacteria is a drug resistant bacteria.

25. The method of claim 21, wherein the method further comprises administering a second therapeutic.

26. The method of claim 25, wherein the second therapeutic is an antibiotic.

27. A method of inhibiting the growth of or killing a bacterial cell, wherein the method comprises contacting the bacterial cell with a composition comprising a compound of claim 1.

28. A method of biosynthesizing a gram-negative active compound, the method comprising:
a) generating a metagenome-derived congener biosynthetic gene cluster (BGC) comprising a nucleic acid molecule, wherein the nucleic acid molecule encodes the gram-negative active compound;
b) providing the nucleic acid compound to a host;
c) incubating the host in a growth medium; and
d) isolating the gram-negative active compound from the host or the growth medium.

29. The method of claim 28, wherein the gram-negative active compound is a compound represented by Formula (I) 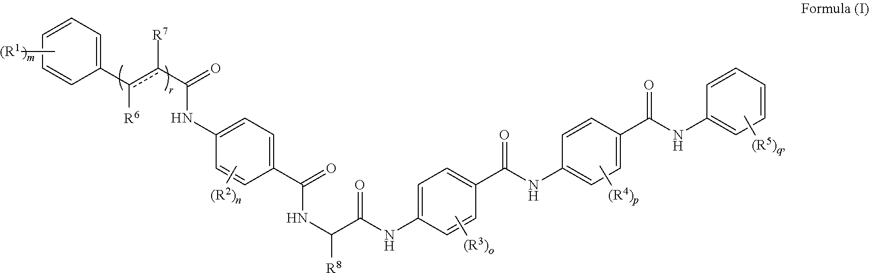
or a racemate, an enantiomer, a diastereomer, a pharmaceutically acceptable salt, or a derivative thereof,
wherein the method comprises:
wherein each R¹, R², R³, R⁴, R⁵, R⁶, R⁷, and R⁸ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof;
each m and q independently represents an integer of 0 to 5;
each n, o, and p independently represents an integer of 0 to 4; and
r represents an integer of 0 or 1.

30. The method of claim 29, wherein each R¹, R², R³, R⁴, R⁵, R⁶, R⁷, and R⁸ is independently selected from the group consisting of hydrogen, alkyl, amino, amido, hydroxyl, hydroxyalkyl, carboxyl, —CN, and any combination thereof.

31. The method of claim 30, wherein each R¹, R², R³, R⁴, R⁵, R⁶, R⁷, and R⁸ is independently selected from the group consisting of hydrogen, methyl, amino, hydroxyl, carboxyl,

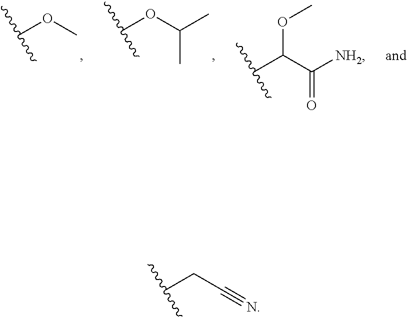

32. The method of claim 29, wherein the compound represented by Formula (I) is a compound selected from the group consisting of: 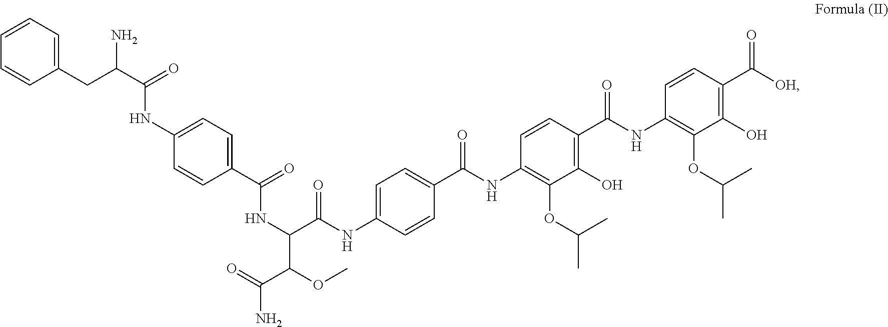 [SMILES: COC(C(N)=O)C(NC(=O)c1ccc(NC(=O)C(N)Cc2ccccc2)cc1)C(=O)Nc1ccc(C(=O)Nc2ccc(C(=O)Nc3ccc(C(=O)O)c(O)c3OC(C)C)c(O)c2OC(C)C)cc1]
or a racemate, an enantiomer, a diastereomer, a pharmaceutically acceptable salt, or a derivative thereof, 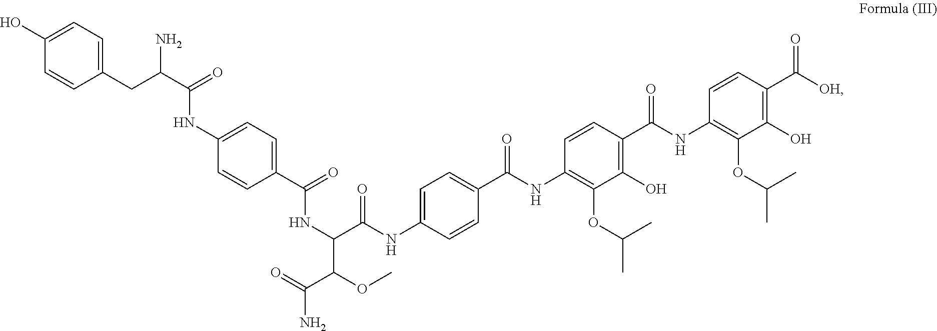 [SMILES: COC(C(N)=O)C(NC(=O)c1ccc(NC(=O)C(N)Cc2ccc(O)cc2)cc1)C(=O)Nc1ccc(C(=O)Nc2ccc(C(=O)Nc3ccc(C(=O)O)c(O)c3OC(C)C)c(O)c2OC(C)C)cc1]
or a racemate, an enantiomer, a diastereomer, a pharmaceutically acceptable salt, or a derivative thereof, 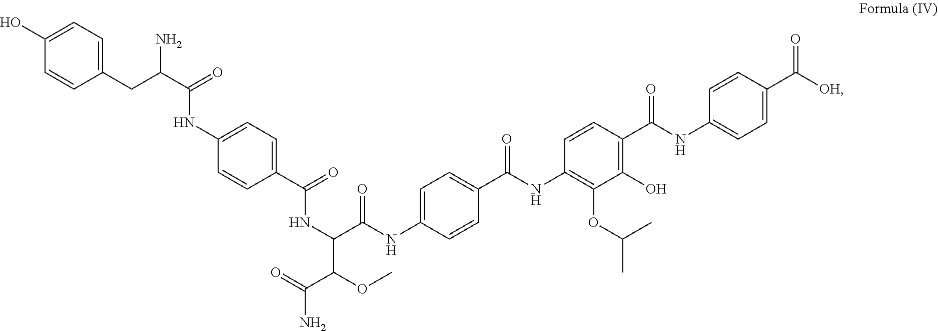 [SMILES: COC(C(N)=O)C(NC(=O)c1ccc(NC(=O)C(N)Cc2ccc(O)cc2)cc1)C(=O)Nc1ccc(C(=O)Nc2ccc(C(=O)Nc3ccc(C(=O)O)cc3)c(O)c2OC(C)C)cc1]
or a racemate, an enantiomer, a diastereomer, a pharmaceutically acceptable salt, or a derivative thereof, 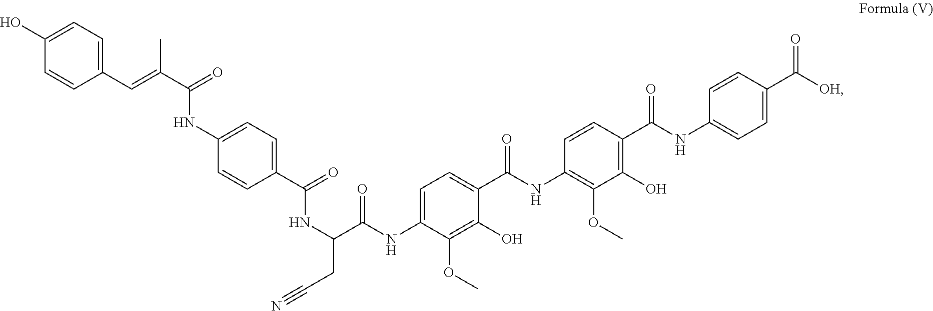 [SMILES: COc1c(NC(=O)c2ccc(NC(=O)C(CC#N)NC(=O)c3ccc(NC(=O)/C(C)=C/c4ccc(O)cc4)cc3)c(OC)c2O)ccc(C(=O)Nc2ccc(C(=O)O)cc2)c1O]
or a racemate, an enantiomer, a diastereomer, a pharmaceutically acceptable salt, or a derivative thereof, 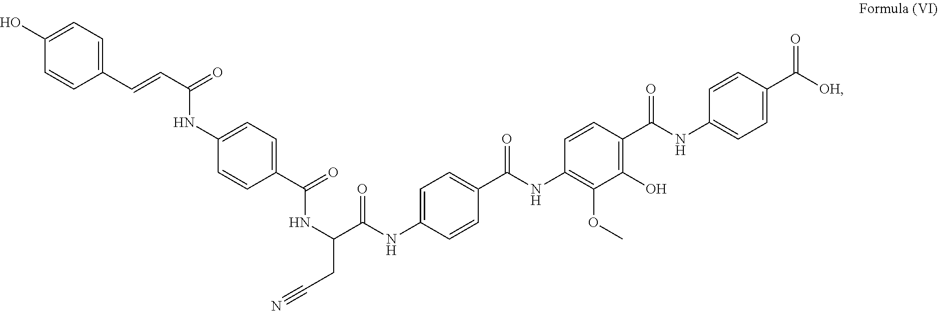 [SMILES: COc1c(NC(=O)c2ccc(NC(=O)C(CC#N)NC(=O)c3ccc(NC(=O)/C=C/c4ccc(O)cc4)cc3)cc2)ccc(C(=O)Nc2ccc(C(=O)O)cc2)c1O]
or a racemate, an enantiomer, a diastereomer, a pharmaceutically acceptable salt, or a derivative thereof, 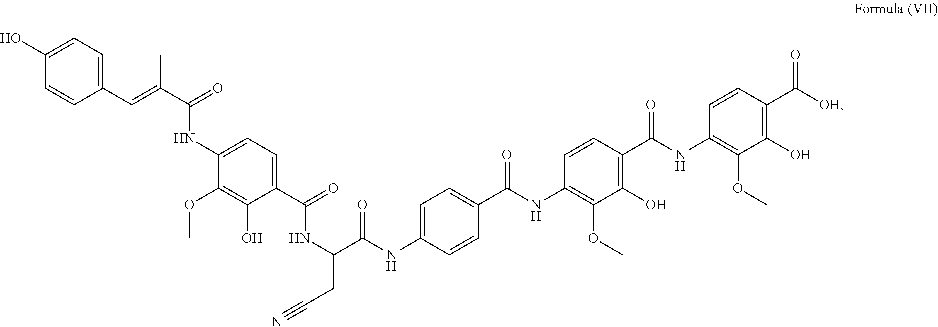 [SMILES: COc1c(NC(=O)c2ccc(NC(=O)c3ccc(NC(=O)C(CC#N)NC(=O)c4ccc(NC(=O)/C(C)=C/c5ccc(O)cc5)c(OC)c4O)cc3)c(OC)c2O)ccc(C(=O)O)c1O]
or a racemate, an enantiomer, a diastereomer, a pharmaceutically acceptable salt, or a derivative thereof, 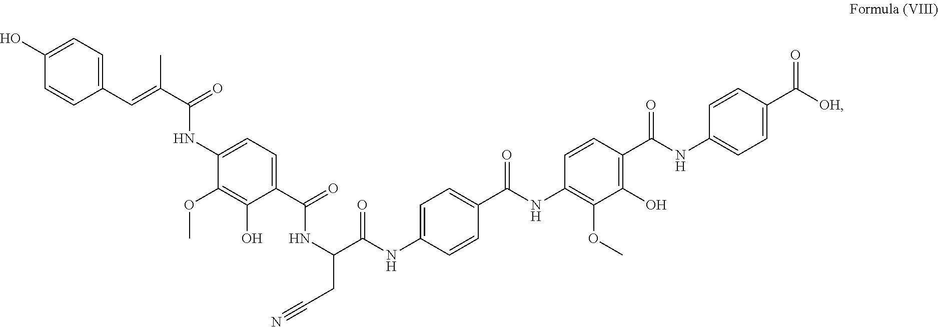 [SMILES: COc1c(NC(=O)/C(C)=C/c2ccc(O)cc2)ccc(C(=O)NC(CC#N)C(=O)Nc2ccc(C(=O)Nc3ccc(C(=O)Nc4ccc(C(=O)O)cc4)c(O)c3OC)cc2)c1O]
or a racemate, an enantiomer, a diastereomer, a pharmaceutically acceptable salt, or a derivative thereof, and 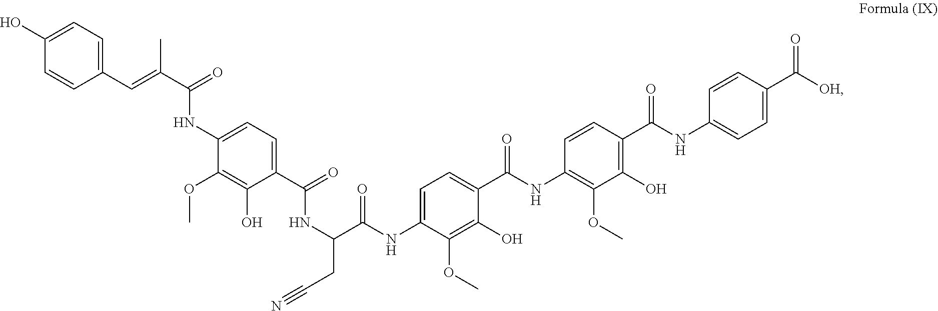 [SMILES: COc1c(NC(=O)/C(C)=C/c2ccc(O)cc2)ccc(C(=O)NC(CC#N)C(=O)Nc2ccc(C(=O)Nc3ccc(C(=O)Nc4ccc(C(=O)O)cc4)c(O)c3OC)c(O)c2OC)c1O]
or a racemate, an enantiomer, a diastereomer, a pharmaceutically acceptable salt, or a derivative thereof.

33. The method of claim 29, wherein the compound represented by Formula (I) is a compound selected from the group consisting of: 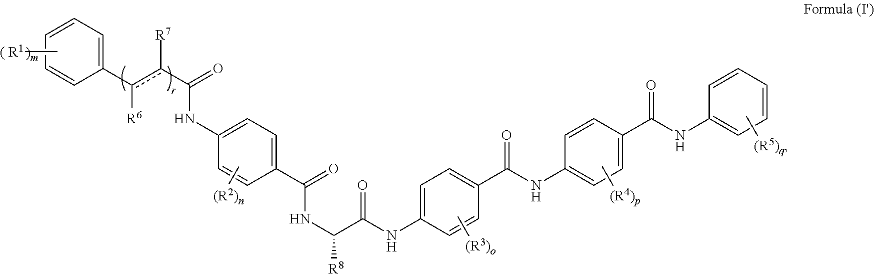
or a racemate, an enantiomer, a diastereomer, a pharmaceutically acceptable salt, or a derivative thereof, and 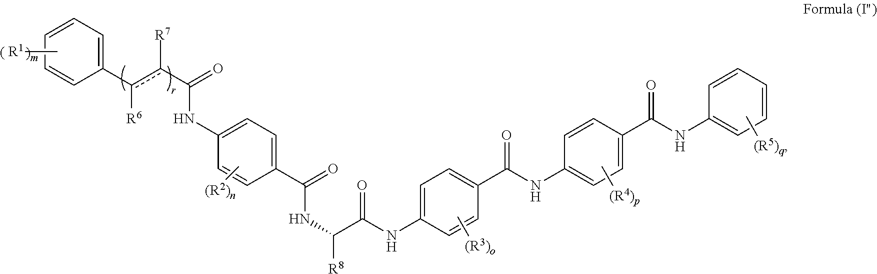
or a racemate, an enantiomer, a diastereomer, a pharmaceutically acceptable salt, or a derivative thereof,
wherein each R¹, R², R³, R⁴, R⁵, R⁶, R⁷, and R⁸ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof;
each m and q independently represents an integer of 0 to 5;
each n, o, and p independently represents an integer of 0 to 4; and
r represents an integer of 0 or 1.

34. The method of claim 33, wherein each R¹, R², R³, R⁴, R⁵, R⁶, R⁷, and R⁸ is independently selected from the group consisting of hydrogen, alkyl, amino, amido, hydroxyl, hydroxyalkyl, carboxyl, —CN, and any combination thereof.

35. The method of claim 34, wherein each R¹, R², R³, R⁴, R⁵, R⁶, R⁷, and R⁸ is independently selected from the group consisting of hydrogen, methyl, amino, hydroxyl, carboxyl

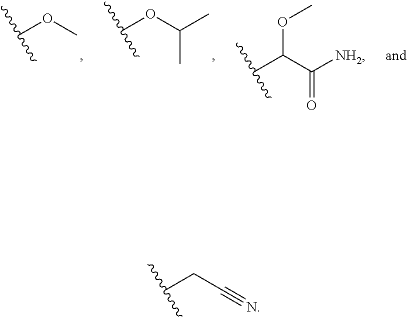

36. The method of claim 29, wherein the compound represented by Formula (I) is a compound selected from the group consisting of: 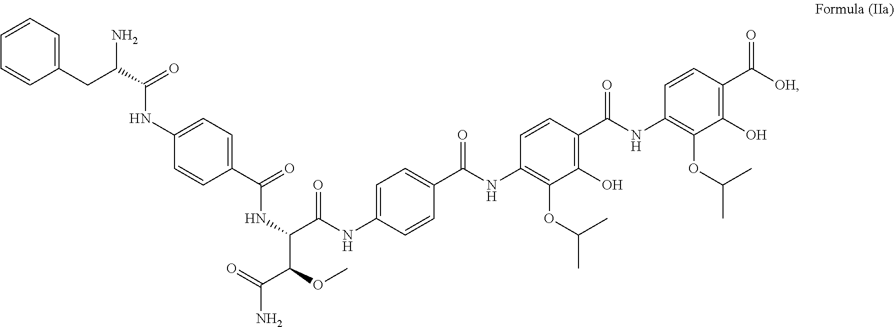
or a racemate, an enantiomer, a diastereomer, a pharmaceutically acceptable salt, or a derivative thereof, 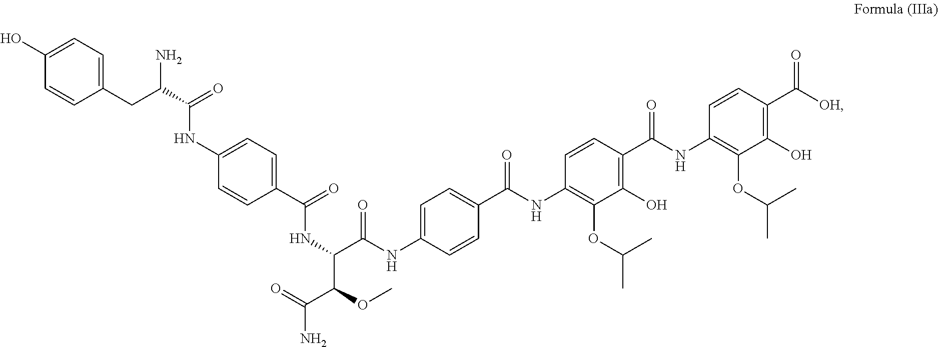
or a racemate, an enantiomer, a diastereomer, a pharmaceutically acceptable salt, or a derivative thereof, 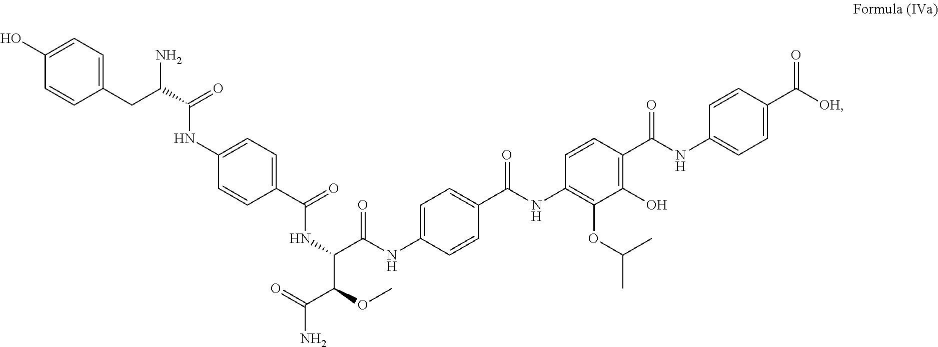
or a racemate, an enantiomer, a diastereomer, a pharmaceutically acceptable salt, or a derivative thereof, 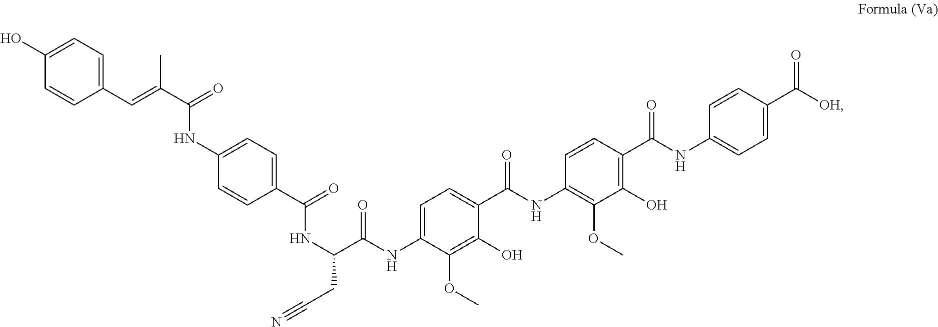 [SMILES: COc1c(NC(=O)c2ccc(NC(=O)[C@H](CC#N)NC(=O)c3ccc(NC(=O)/C(C)=C/c4ccc(O)cc4)cc3)c(OC)c2O)ccc(C(=O)Nc2ccc(C(=O)O)cc2)c1O]
or a racemate, an enantiomer, a diastereomer, a pharmaceutically acceptable salt, or a derivative thereof, 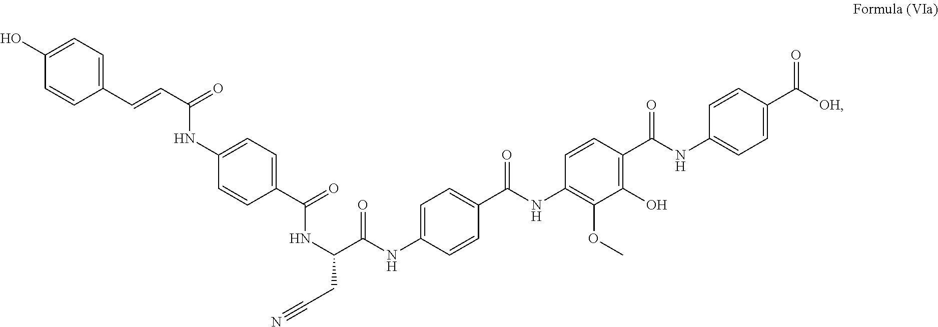 [SMILES: COc1c(NC(=O)c2ccc(NC(=O)[C@H](CC#N)NC(=O)c3ccc(NC(=O)/C=C/c4ccc(O)cc4)cc3)cc2)ccc(C(=O)Nc2ccc(C(=O)O)cc2)c1O]
or a racemate, an enantiomer, a diastereomer, a pharmaceutically acceptable salt, or a derivative thereof, 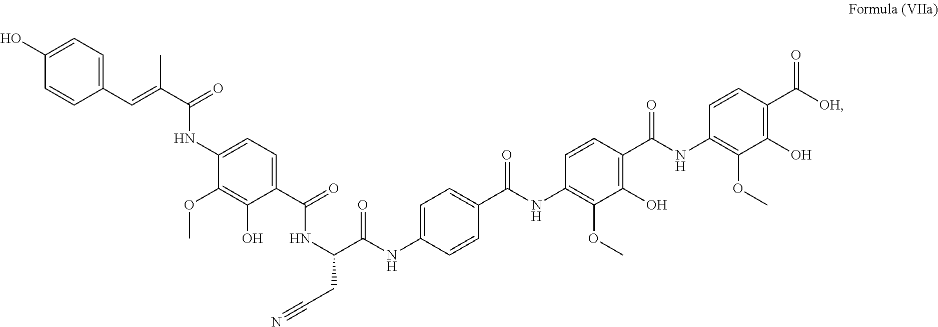 [SMILES: COc1c(NC(=O)c2ccc(NC(=O)c3ccc(NC(=O)[C@H](CC#N)NC(=O)c4ccc(NC(=O)/C(C)=C/c5ccc(O)cc5)c(OC)c4O)cc3)c(OC)c2O)ccc(C(=O)O)c1O]
or a racemate, an enantiomer, a diastereomer, a pharmaceutically acceptable salt, or a derivative thereof, 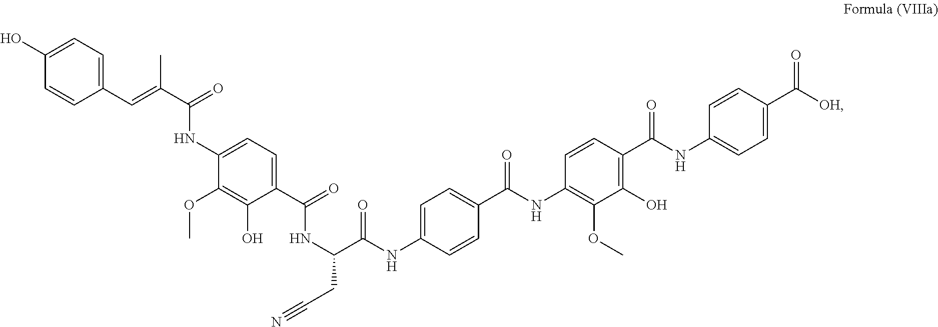 [SMILES: COc1c(NC(=O)/C(C)=C/c2ccc(O)cc2)ccc(C(=O)N[C@@H](CC#N)C(=O)Nc2ccc(C(=O)Nc3ccc(C(=O)Nc4ccc(C(=O)O)cc4)c(O)c3OC)cc2)c1O]
or a racemate, an enantiomer, a diastereomer, a pharmaceutically acceptable salt, or a derivative thereof, and 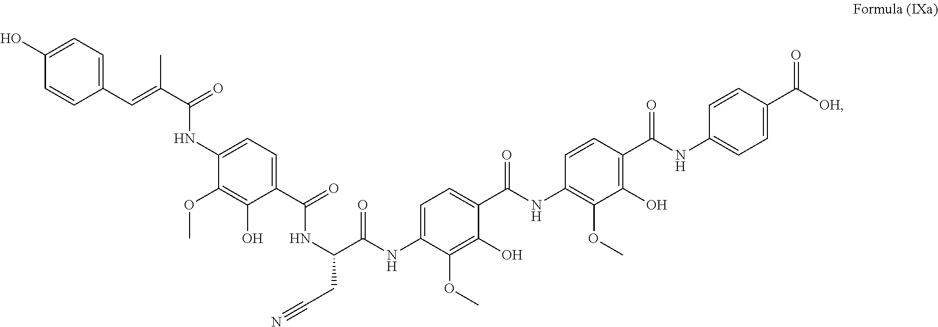 [SMILES: COc1c(NC(=O)/C(C)=C/c2ccc(O)cc2)ccc(C(=O)N[C@@H](CC#N)C(=O)Nc2ccc(C(=O)Nc3ccc(C(=O)Nc4ccc(C(=O)O)cc4)c(O)c3OC)c(O)c2OC)c1O]
or a racemate, an enantiomer, a diastereomer, a pharmaceutically acceptable salt, or a derivative thereof.

37. A method of generating a compound represented by Formula (I) 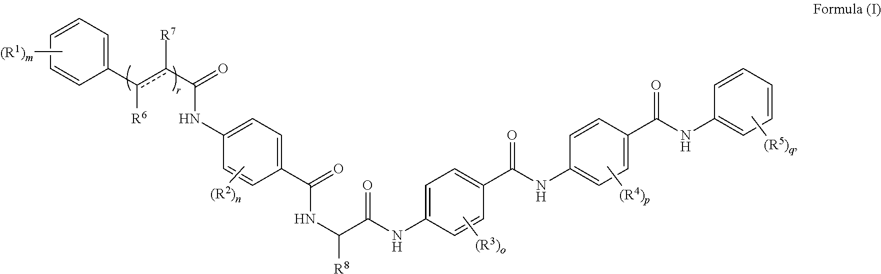
or a racemate, an enantiomer, a diastereomer, a pharmaceutically acceptable salt, or a derivative thereof,
wherein the method comprises:
a) reacting two or more compounds selected from the group consisting of: 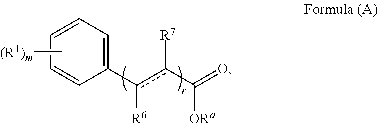
or a racemate, an enantiomer, a diastereomer, a pharmaceutically acceptable salt, or a derivative thereof, 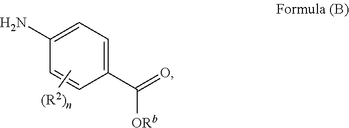
or a racemate, an enantiomer, a diastereomer, a pharmaceutically acceptable salt, or a derivative thereof, 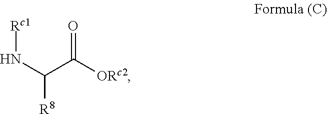
or a racemate, an enantiomer, a diastereomer, a pharmaceutically acceptable salt, or a derivative thereof, 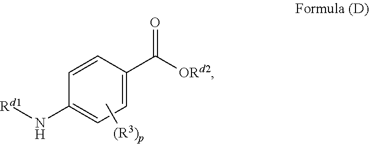
or a racemate, an enantiomer, a diastereomer, a pharmaceutically acceptable salt, or a derivative thereof, 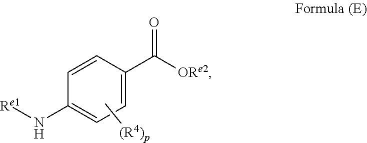
or a racemate, an enantiomer, a diastereomer, a pharmaceutically acceptable salt, or a derivative thereof, and 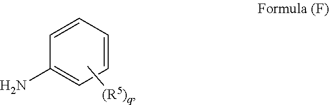
or a racemate, an enantiomer, a diastereomer, a pharmaceutically acceptable salt, or a derivative thereof;
wherein each R¹, R², R³, R⁴, R⁵, R⁶, R⁷, and R⁸ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof;
each R^{a}, R^{b}, R^{c1}, R^{c2}, R^{d1}, R^{d2}, R^{e1}, and R^{e2} is independently selected from the group consisting of hydrogen, deuterium, halogen, alkyl, allyl, protecting group, an amino acid, and any combinations thereof,
each m and q independently represents an integer of 0 to 5;
each n, o, and p independently represents an integer of 0 to 4; and
r represents an integer of 0 or 1.
